SEQUENCE LISTING (158 sequences)

SEQ ID NO: 1 - protein, 122 aa, synthetic construct
  REGION 1..122: note = VH
QVQLQQSGAELVKPGASVKLSCKASGYTFTRYYLYWVKQRPGQGLEWIGEINPNNGGTKFNEKFKSKATLTVDKSSRTTYIQLSSLTSEDSAVYYCSREDDYDGTPDAMDYWGQGTAVTVSS

SEQ ID NO: 2 - protein, 5 aa, synthetic construct
  REGION 1..5: note = CDRH1
RYYLY

SEQ ID NO: 3 - protein, 17 aa, synthetic construct
  REGION 1..17: note = CDRH2
EINPNNGGTKFNEKFKS

SEQ ID NO: 4 - protein, 13 aa, synthetic construct
  REGION 1..13: note = CDRH3
EDDYDGTPDAMDY

SEQ ID NO: 5 - protein, 107 aa, synthetic construct
  REGION 1..107: note = VL
DIQMNQSPSSLSASLGDTITITCHASQNINLWLSWYQQRPGNIPKLLIYRASNLHTGVPSRFSGSGSATGFTLTISSLQPEDIATYYCQQGHSYPYTFGGGTKLDIK

SEQ ID NO: 6 - protein, 11 aa, synthetic construct
  REGION 1..11: note = CDRL1
HASQNINLWLS

SEQ ID NO: 7 - protein, 7 aa, synthetic construct
  REGION 1..7: note = CDRL2
RASNLHT

SEQ ID NO: 8 - protein, 8 aa, synthetic construct
  REGION 1..8: note = CDRL3
QGHSYPYT

SEQ ID NO: 9 - protein, 735 aa, synthetic construct
  REGION 1..735: note = Heavy chain protein with leader
MGWSYIILFLVTTATGVHSQVQLQQSGAELVKPGASVKLSCKASGYTFTRYYLYWVKQRPGQGLEWIGEINPNNGGTKFNEKFKSKATLTVDKSSRTTYIQLSSLTSEDSAVYYCSREDDYDGTPDAMDYWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKGGGGSGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 10 - protein, 110 aa, synthetic construct
  REGION 1..110: note = VL
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 11 - protein, 12 aa, synthetic construct
  REGION 1..12: note = CDRL1
SVSSSISSNNLH

SEQ ID NO: 12 - protein, 7 aa, synthetic construct
  REGION 1..7: note = CDRL2
GTSNLAS

SEQ ID NO: 13 - protein, 11 aa, synthetic construct
  REGION 1..11: note = CDRL3
QQWSSYPYMYT

SEQ ID NO: 14 - protein, 119 aa, synthetic construct
  REGION 1..119: note = VH
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 15 - protein, 10 aa, synthetic construct
  REGION 1..10: note = CDRH1
GFTFSGYGLS

SEQ ID NO: 16 - protein, 17 aa, synthetic construct
  REGION 1..17: note = CDRH2
MISSGGSYTYYADSVKG

SEQ ID NO: 17 - protein, 10 aa, synthetic construct
  REGION 1..10: note = CDRH3
HGDDPAWFAY

SEQ ID NO: 18 - protein, 113 aa, synthetic construct
  REGION 1..113: note = VL
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 19 - protein, 14 aa, synthetic construct
  REGION 1..14: note = CDRL1
QSLLYRSNQKNYLA

SEQ ID NO: 20 - protein, 10 aa, synthetic construct
  REGION 1..10: note = CDRL2
LIYWASTRES

SEQ ID NO: 21 - protein, 8 aa, synthetic construct
  REGION 1..8: note = CDRL3
QQYYNYPR

SEQ ID NO: 22 - protein, 124 aa, synthetic construct
  REGION 1..124: note = VH
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 23 - protein, 10 aa, synthetic construct
  REGION 1..10: note = CDRH1
YSITSDYAWN

SEQ ID NO: 24 - protein, 13 aa, synthetic construct
  REGION 1..13: note = CDRH2
WVGYISNSGSTSY

SEQ ID NO: 25 - protein, 16 aa, synthetic construct
  REGION 1..16: note = CDRH3
RERNYDYDDYYYAMDY

SEQ ID NO: 26 - protein, 108 aa, synthetic construct
  REGION 1..108: note = VL
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIK

SEQ ID NO: 27 - protein, 12 aa, synthetic construct
  REGION 1..12: note = CDRL1
RASQRVNNNYLA

SEQ ID NO: 28 - protein, 7 aa, synthetic construct
  REGION 1..7: note = CDRL2
GASSRAT

SEQ ID NO: 29 - protein, 9 aa, synthetic construct
  REGION 1..9: note = CDRL3
QQYDRSPLT

SEQ ID NO: 30 - protein, 118 aa, synthetic construct
  REGION 1..118: note = VH
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSS

SEQ ID NO: 31 - protein, 5 aa, synthetic construct
  REGION 1..5: note = CDRH1
SYYWS

SEQ ID NO: 32 - protein, 16 aa, synthetic construct
  REGION 1..16: note = CDRH2
YVYYSGTTNYNPSLKS

SEQ ID NO: 33 - protein, 10 aa, synthetic construct
  REGION 1..10: note = CDRH3
IAVTGFYFDY

SEQ ID NO: 34 - protein, 113 aa, synthetic construct
  REGION 1..113: note = VL
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 35 - protein, 14 aa, synthetic construct
  REGION 1..14: note = CDRL1
QSLLNSGNQKNYLT

SEQ ID NO: 36 - protein, 10 aa, synthetic construct
  REGION 1..10: note = CDRL2
LIYWASTRES

SEQ ID NO: 37 - protein, 8 aa, synthetic construct
  REGION 1..8: note = CDRL3
QNDYSYPF

SEQ ID NO: 38 - protein, 118 aa, synthetic construct
  REGION 1..118: note = VH
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 39 - protein, 9 aa, synthetic construct
  REGION 1..9: note = CDRH1
YTFTSYWIN

SEQ ID NO: 40 - protein, 14 aa, synthetic construct
  REGION 1..14: note = CDRH2
WIGNIYPSDSYTNY

SEQ ID NO: 41 - protein, 10 aa, synthetic construct
  REGION 1..10: note = CDRH3
RSWRGNSFDY

SEQ ID NO: 42 - protein, 122 aa, synthetic construct
  REGION 1..122: note = VH-N185S-K190N
QVQLQQSGAELVKPGASVKLSCKASGYTFTRYYLYWVKQRPGQGLEWIGEINPSNGGTNFNEKFKSKATLTVDKSSRTTYIQLSSLTSEDSAVYYCSREDDYDGTPDAMDYWGQGTAVTVSS

SEQ ID NO: 43 - protein, 5 aa, synthetic construct
  REGION 1..5: note = CDRH1
RYYLY

SEQ ID NO: 44 - protein, 17 aa, synthetic construct
  REGION 1..17: note = CDRH2
EINPSNGGTNFNEKFKS

SEQ ID NO: 45 - protein, 13 aa, synthetic construct
  REGION 1..13: note = CDRH3
EDDYDGTPDAMDY

SEQ ID NO: 46 - protein, 122 aa, synthetic construct
  REGION 1..122: note = VH-R162S-Y164W
QVQLQQSGAELVKPGASVKLSCKASGYTFTSYWLYWVKQRPGQGLEWIGEINPNNGGTKFNEKFKSKATLTVDKSSRTTYIQLSSLTSEDSAVYYCSREDDYDGTPDAMDYWGQGTAVTVSS

SEQ ID NO: 47 - protein, 5 aa, synthetic construct
  REGION 1..5: note = CDRH1
SYWLY

SEQ ID NO: 48 - protein, 17 aa, synthetic construct
  REGION 1..17: note = CDRH2
EINPNNGGTKFNEKFKS

SEQ ID NO: 49 - protein, 13 aa, synthetic construct
  REGION 1..13: note = CDRH3
EDDYDGTPDAMDY

SEQ ID NO: 50 - protein, 122 aa, synthetic construct
  REGION 1..122: note = VH-cluster1-2
QVQLQQSGAELVKPGASVKLSCKASGYTFTSYWMHWVKQRPGQGLEWIGEINPSNGRTNYNEKFKSKATLTVDKSSRTTYIQLSSLTSEDSAVYYCSREDDYDGTPDAMDYWGQGTAVTVSS

SEQ ID NO: 51 - protein, 5 aa, synthetic construct
  REGION 1..5: note = CDRH1
SYWMH

SEQ ID NO: 52 - protein, 17 aa, synthetic construct
  REGION 1..17: note = CDRH2
EINPSNGRTNYNEKFKS

SEQ ID NO: 53 - protein, 13 aa, synthetic construct
  REGION 1..13: note = CDRH3
EDDYDGTPDAMDY

SEQ ID NO: 54 - protein, 122 aa, synthetic construct
  REGION 1..122: note = VH-cluster1
QVQLQQSGAELVKPGASVKLSCKASGYTFTSYWMHWVKQRPGQGLEWIGEINPNNGGTKFNEKFKSKATLTVDKSSRTTYIQLSSLTSEDSAVYYCSREDDYDGTPDAMDYWGQGTAVTVSS

SEQ ID NO: 55 - protein, 5 aa, synthetic construct
  REGION 1..5: note = CDRH1
SYWMH

SEQ ID NO: 56 - protein, 17 aa, synthetic construct
  REGION 1..17: note = CDRH2
EINPNNGGTKFNEKFKS

SEQ ID NO: 57 - protein, 13 aa, synthetic construct
  REGION 1..13: note = CDRH3
EDDYDGTPDAMDY

SEQ ID NO: 58 - protein, 122 aa, synthetic construct
  REGION 1..122: note = VH-cluster2
QVQLQQSGAELVKPGASVKLSCKASGYTFTRYYLYWVKQRPGQGLEWIGEINPSNGRTNYNEKFKSKATLTVDKSSRTTYIQLSSLTSEDSAVYYCSREDDYDGTPDAMDYWGQGTAVTVSS

SEQ ID NO: 59 - protein, 5 aa, synthetic construct
  REGION 1..5: note = CDRH1
RYYLY

SEQ ID NO: 60 - protein, 17 aa, synthetic construct
  REGION 1..17: note = CDRH2
EINPSNGRTNYNEKFKS

SEQ ID NO: 61 - protein, 13 aa, synthetic construct
  REGION 1..13: note = CDRH3
EDDYDGTPDAMDY

SEQ ID NO: 62 - protein, 122 aa, synthetic construct
  REGION 1..122: note = VH-46_1A
QVQLVQSGAEVKKPGASVKVSCKASGYTFTRYYLYWVRQAPGQGLEWIGEINPSNGGTNFNEKFKSRVTLTVDKSTRTTYIELSSLRSEDTAVYYCSREDDYDGTPDAMDYWGQGTLVTVSS

SEQ ID NO: 63 - protein, 5 aa, synthetic construct
  REGION 1..5: note = CDRH1
RYYLY

SEQ ID NO: 64 - protein, 17 aa, synthetic construct
  REGION 1..17: note = CDRH2
EINPSNGGTNFNEKFKS

SEQ ID NO: 65 - protein, 13 aa, synthetic construct
  REGION 1..13: note = CDRH3
EDDYDGTPDAMDY

SEQ ID NO: 66 - protein, 122 aa, synthetic construct
  REGION 1..122: note = VH-46_1B
QVQLVQSGAEVKKPGASVKVSCKASGYTFTRYYMYWVRQAPGQGLEWMGEINPSNGGTNFAQKFQGRVTMTVDKSTSTVYMELSSLRSEDTAVYYCSREDDYDGTPDAMDYWGQGTLVTVSS

SEQ ID NO: 67 - protein, 5 aa, synthetic construct
  REGION 1..5: note = CDRH1
RYYMY

SEQ ID NO: 68 - protein, 17 aa, synthetic construct
  REGION 1..17: note = CDRH2
EINPSNGGTNFAQKFQG

SEQ ID NO: 69 - protein, 13 aa, synthetic construct
  REGION 1..13: note = CDRH3
EDDYDGTPDAMDY

SEQ ID NO: 70 - protein, 122 aa, synthetic construct
  REGION 1..122: note = VH-34_01A
QVQLQQSGAGLLKPSETLSLTCAAYGYTFTRYYLYWVRQPPGKGLEWIGEINPSNGGTNFNEKLKSRVTLSVDKSKRQTSIKLSSVTAADTAVYYCSREDDYDGTPDAMDYWGQGTLVTVSS

SEQ ID NO: 71 - protein, 5 aa, synthetic construct
  REGION 1..5: note = CDRH1
RYYLY

SEQ ID NO: 72 - protein, 17 aa, synthetic construct
  REGION 1..17: note = CDRH2
EINPSNGGTNFNEKLKS

SEQ ID NO: 73 - protein, 13 aa, synthetic construct
  REGION 1..13: note = CDRH3
EDDYDGTPDAMDY

SEQ ID NO: 74 - protein, 107 aa, synthetic construct
  REGION 1..107: note = Light Chain - L31V-R39K-R50K-A68G-H92Q-Y96L
DIQMNQSPSSLSASLGDTITITCHASQNINVWLSWYQQKPGNIPKLLIYKASNLHTGVPSRFSGSGSGTGFTLTISSLQPEDIATYYCQQGQSYPLTFGGGTKLDIK

SEQ ID NO: 75 - protein, 11 aa, synthetic construct
  REGION 1..11: note = CDRL1
HASQNINVWLS

SEQ ID NO: 76 - protein, 7 aa, synthetic construct
  REGION 1..7: note = CDRL2
KASNLHT

SEQ ID NO: 77 - protein, 8 aa, synthetic construct
  REGION 1..8: note = CDRL3
QGQSYPLT

SEQ ID NO: 78 - protein, 107 aa, synthetic construct
  REGION 1..107: note = Light Chain - L31V-H92Q-Y96L
DIQMNQSPSSLSASLGDTITITCHASQNINVWLSWYQQRPGNIPKLLIYRASNLHTGVPSRFSGSGSATGFTLTISSLQPEDIATYYCQQGQSYPLTFGGGTKLDIK

SEQ ID NO: 79 - protein, 11 aa, synthetic construct
  REGION 1..11: note = CDRL1
HASQNINVWLS

SEQ ID NO: 80 - protein, 7 aa, synthetic construct
  REGION 1..7: note = CDRL2
RASNLHT

SEQ ID NO: 81 - protein, 8 aa, synthetic construct
  REGION 1..8: note = CDRL3
QGQSYPLT

SEQ ID NO: 82 - protein, 107 aa, synthetic construct
  REGION 1..107: note = Light Chain H92Q-Y96L
DIQMNQSPSSLSASLGDTITITCHASQNINLWLSWYQQRPGNIPKLLIYRASNLHTGVPSRFSGSGSATGFTLTISSLQPEDIATYYCQQGQSYPLTFGGGTKLDIK

SEQ ID NO: 83 - protein, 11 aa, synthetic construct
  REGION 1..11: note = CDRL1
HASQNINLWLS

SEQ ID NO: 84 - protein, 7 aa, synthetic construct
  REGION 1..7: note = CDRL2
RASNLHT

SEQ ID NO: 85 - protein, 8 aa, synthetic construct
  REGION 1..7: note = CDRL3
QGQSYPLT

SEQ ID NO: 86 - protein, 107 aa, synthetic construct
  REGION 1..107: note = Light Chain - L31V
DIQMNQSPSSLSASLGDTITITCHASQNINVWLSWYQQRPGNIPKLLIYRASNLHTGVPSRFSGSGSATGFTLTISSLQPEDIATYYCQQGHSYPYTFGGGTKLDIK

SEQ ID NO: 87 - protein, 11 aa, synthetic construct
  REGION 1..11: note = CDRL1
HASQNINVWLS

SEQ ID NO: 88 - protein, 7 aa, synthetic construct
  REGION 1..7: note = CDRL2
RASNLHT

SEQ ID NO: 89 - protein, 8 aa, synthetic construct
  REGION 1..8: note = CDRL3
QGHSYPYT

SEQ ID NO: 90 - protein, 118 aa, synthetic construct
  REGION 1..118: note = VH
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 91 - protein, 108 aa, synthetic construct
  REGION 1..108: note = VL
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 92 - protein, 743 aa, synthetic construct
  REGION 1..743: note = EvB#3 Heavy chain
MGWSYIILFLVTTATGVHSQVQLQQSGAELVKPGASVKLSCKASGYTFTRYYLYWVKQRPGQGLEWIGEINPNNGGTKFNEKFKSKATLTVDKSSRTTYIQLSSLTSEDSAVYYCSREDDYDGTPDAMDYWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 93 - protein, 234 aa, synthetic construct
  REGION 1..234: note = EvB3# Light chain
MRVLAELLGLLLFCFLGVRCDIQMNQSPSSLSASLGDTITITCHASQNINLWLSWYQQRPGNIPKLLIYRASNLHTGVPSRFSGSGSATGFTLTISSLQPEDIATYYCQQGHSYPYTFGGGTKLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 471 aa, synthetic construct
  REGION 1..471: note = HC
MGWSYIILFLVTTATGVHSQVQLQQSGAELVKPGASVKLSCKASGYTFTRYYLYWVKQRPGQGLEWIGEINPNNGGTKFNEKFKSKATLTVDKSSRTTYIQLSSLTSEDSAVYYCSREDDYDGTPDAMDYWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 19 aa, synthetic construct
  REGION 1..19: note = reference HC leader
MGWSYIILFLVTTATGVHS

SEQ ID NO: 96 - RNA, 20 nt, synthetic construct
  misc_feature 1..20: note = RNA Sequence
  source 1..20: mol_type = other RNA
caaggaaaagccaggccccg

SEQ ID NO: 97 - protein, 15 aa, synthetic construct
  REGION 1..15: note = Linker 1
GGGGSGGGGSGGGGS

SEQ ID NO: 98 - protein, 20 aa, synthetic construct
  REGION 1..20: note = Linker 2
GGGGSGGGGSGGGGSGGGGS

SEQ ID NO: 99 - protein, 12 aa, synthetic construct
  REGION 1..12: note = Linker 3
GSAPAPAPAPAP

SEQ ID NO: 100 - protein, 10 aa, synthetic construct
  REGION 1..10: note = Linker 4
APAPAPAPAP

SEQ ID NO: 101 - protein, 20 aa, synthetic construct
  REGION 1..20: note = Linker 5
APAPAPAPAPAPAPAPAPAP

SEQ ID NO: 102 - protein, 732 aa, synthetic construct
  REGION 1..732: note = EvB#4-HC
MGWSYIILFLVTTATGVHSQVQLQQSGAELVKPGASVKLSCKASGYTFTRYYLYWVKQRPGQGLEWIGEINPNNGGTKFNEKFKSKATLTVDKSSRTTYIQLSSLTSEDSAVYYCSREDDYDGTPDAMDYWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKGGGGSGGGGSGGGGSGGGGSQVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSS

SEQ ID NO: 103 - protein, 737 aa, synthetic construct
  REGION 1..737: note = EvB#6-HC
MGWSYIILFLVTTATGVHSQVQLQQSGAELVKPGASVKLSCKASGYTFTRYYLYWVKQRPGQGLEWIGEINPNNGGTKFNEKFKSKATLTVDKSSRTTYIQLSSLTSEDSAVYYCSREDDYDGTPDAMDYWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKGGGGSGGGGSGGGGSGGGGSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 104 - protein, 122 aa, synthetic construct
QVQLQQSGAGLLKPSETLSLTCAAYGGTFTRYYWYWVRQPPGKGLEWIGEINPSNGGTNFNEKLKSRVTLSVDKSKRQTSIKLSSVTAADTAVYYCSREDDYDGTPDAMDYWGQGTLVTVSS

SEQ ID NO: 105 - protein, 5 aa, synthetic construct
RYYWY

SEQ ID NO: 106 - protein, 17 aa, synthetic construct
EINPSNGGTNFNEKLKS

SEQ ID NO: 107 - protein, 13 aa, synthetic construct
EDDYDGTPDAMDY

SEQ ID NO: 108 - protein, 122 aa, synthetic construct
QVQLQQSGAGLLKPSETLSLTCAAYGYTFSRYYLYWIRQPPGKGLEWIGEINPSNGGTNFNESLKSRVTISVDKSKNQTSLKLSSVTAADTAVYYCSREDDYDGTPDAMDYWGQGTLVTVSS

SEQ ID NO: 109 - protein, 5 aa, synthetic construct
RYYLY

SEQ ID NO: 110 - protein, 17 aa, synthetic construct
EINPSNGGTNFNESLKS

SEQ ID NO: 111 - protein, 13 aa, synthetic construct
EDDYDGTPDAMDY

SEQ ID NO: 112 - protein, 122 aa, synthetic construct
QVQLQQSGAGLLKPSETLSLTCAAYGGTFSRYYWYWIRQPPGKGLEWIGEINPSNGGTNFNESLKSRVTISVDKSKNQTSLKLSSVTAADTAVYYCSREDDYDGTPDAMDYWGQGTLVTVSS

SEQ ID NO: 113 - protein, 5 aa, synthetic construct
RYYWY

SEQ ID NO: 114 - protein, 17 aa, synthetic construct
EINPSNGGTNFNESLKS

SEQ ID NO: 115 - protein, 13 aa, synthetic construct
EDDYDGTPDAMDY

SEQ ID NO: 116 - protein, 107 aa, synthetic construct
DIQMTQSPSSVSASVGDRVTITCHASQNINLWLSWYQQKPGKAPKLLIYRASNLHTGVPSRFSGSGSATDFTLTISSLQPEDFATYYCQQGHSYPYTFGQGTKLEIK

SEQ ID NO: 117 - protein, 11 aa, synthetic construct
HASQNINLWLS

SEQ ID NO: 118 - protein, 7 aa, synthetic construct
RASNLHT

SEQ ID NO: 119 - protein, 8 aa, synthetic construct
QGHSYPYT

SEQ ID NO: 120 - protein, 107 aa, synthetic construct
DIQMTQSPSSVSASVGDRVTITCRASQGISSWLSWYQQKPGKAPKLLIYRASNLHTGVPSRFSGSGSATDFTLTISSLQPEDFATYYCQQGHSYPYTFGQGTKLEIK

SEQ ID NO: 121 - protein, 11 aa, synthetic construct
RASQGISSWLS

SEQ ID NO: 122 - protein, 7 aa, synthetic construct
RASNLHT

SEQ ID NO: 123 - protein, 8 aa, synthetic construct
QGHSYPYT

SEQ ID NO: 124 - protein, 107 aa, synthetic construct
DIQMTQSPSSVSASVGDRVTITCRASQGISSWLSWYQQKPGKAPKLLIYRASNLHTGVPSRFSGSGSGTDFTLTISSLQPEDFATYYCQQGHSYPYTFGQGTKLEIK

SEQ ID NO: 125 - protein, 11 aa, synthetic construct
RASQGISSWLS

SEQ ID NO: 126 - protein, 7 aa, synthetic construct
RASNLHT

SEQ ID NO: 127 - protein, 8 aa, synthetic construct
QGHSYPYT

SEQ ID NO: 128 - protein, 107 aa, synthetic construct
EIVMTQSPATLSLSPGERATLSCHASQNINLWLSWYQQKPGQAPRLLIYRASNLHTGIPARFSGSGSATDFTLTISSLEPEDFAVYYCQQGHSYPYTFGQGTKLEIK

SEQ ID NO: 129 - protein, 11 aa, synthetic construct
HASQNINLWLS

SEQ ID NO: 130 - protein, 7 aa, synthetic construct
RASNLHT

SEQ ID NO: 131 - protein, 8 aa, synthetic construct
QGHSYPYT

SEQ ID NO: 132 - protein, 107 aa, synthetic construct
EIVLTQSPATLSLSPGERATLSCRASQSVSSWLSWYQQKPGQAPRLLIYRASNLHTGIPARFSGSGSATDFTLTISSLEPEDFAVYYCQQGHSYPYTFGQGTKLEIK

SEQ ID NO: 133 - protein, 11 aa, synthetic construct
RASQSVSSWLS

SEQ ID NO: 134 - protein, 7 aa, synthetic construct
RASNLHT

SEQ ID NO: 135 - protein, 8 aa, synthetic construct
QGHSYPYT

SEQ ID NO: 136 - protein, 107 aa, synthetic construct
EIVLTQSPATLSLSPGERATLSCRASQSVSSWLSWYQQKPGQAPRLLIYRASNRHTGIPARFSGSGSGTDFTLTISSLEPEDFAVYYCQQGHSYPYTFGQGTKLEIK

SEQ ID NO: 137 - protein, 11 aa, synthetic construct
RASQSVSSWLS

SEQ ID NO: 138 - protein, 7 aa, synthetic construct
RASNRHT

SEQ ID NO: 139 - protein, 8 aa, synthetic construct
QGHSYPYT

SEQ ID NO: 140 - protein, 11 aa, synthetic construct
RASQGISVWLS

SEQ ID NO: 141 - protein, 11 aa, synthetic construct
RASQSVSVWLS

SEQ ID NO: 142 - protein, 23 aa, synthetic construct
DIQMTQSPSSVSASVGDRVTITC

SEQ ID NO: 143 - protein, 15 aa, synthetic construct
WYQQKPGKAPKLLIY

SEQ ID NO: 144 - protein, 33 aa, synthetic construct
GVPSRFSGSGSGTDFTLTISSLQPEDFATYYCQ

SEQ ID NO: 145 - protein, 33 aa, synthetic construct
GIPARFSGSGSATDFTLTISSLEPEDFAVYYCQ

SEQ ID NO: 146 - protein, 10 aa, synthetic construct
FGQGTKLEIK

SEQ ID NO: 147 - protein, 30 aa, synthetic construct
QVQLVQSGAEVKKPGASVKVSCKASGYTFT

SEQ ID NO: 148 - protein, 14 aa, synthetic construct
WVRQAPGQGLEWIG

SEQ ID NO: 149 - protein, 32 aa, synthetic construct
RVTISVDKSKNQTSLKLSSVTAADTAVYYCSR

SEQ ID NO: 150 - protein, 11 aa, synthetic construct
WGQGTLVTVSS

SEQ ID NO: 151 - protein, 11 aa, synthetic construct
HASQNINLYLS

SEQ ID NO: 152 - protein, 11 aa, synthetic construct
HASQNINLLLS

SEQ ID NO: 153 - protein, 11 aa, synthetic construct
HASQNINLALS

SEQ ID NO: 154 - protein, 11 aa, synthetic construct
HASQNINTWLS

SEQ ID NO: 155 - protein, 11 aa, unidentified
HASQNINQWLS

SEQ ID NO: 156 - protein, 8 aa, synthetic construct
QGQSYPYT

SEQ ID NO: 157 - protein, 8 aa, synthetic construct
QGHSFPYT

SEQ ID NO: 158 - protein, 8 aa, synthetic construct
QGHSYPLT

(19) United States
(12) Patent Application Publication
(10) Pub. No.: US 2025/0163157 A1
(43) Pub. Date: May 22, 2025

(54) BISPECIFIC ANTIBODIES AGAINST CD277 AND A TUMOR-ANTIGEN

(71) Applicant: Boehringer Ingelheim International GmbH, Ingelheim am Rhein (DE)

(72) Inventors: Christoph Baumann, Brunn am Gebirge (AT); Klaus-Peter Kuenkele, Voesendorf (AT); Hans-Heinrich Oberg, Kiel (DE); Matthias Peipp, Hamburg (DE); Daniela Wesch, Kiel (DE); Sandeep Kumar, Ridgefield, CT (US); Tadas Panavas, Ridgefield, CT (US); Nicolas Sabarth, Ingelheim am Rhein (DE)

(21) Appl. No.: 18/841,269

(22) PCT Filed: Feb. 26, 2023
(86) PCT No.: PCT/EP2023/054762

§ 371 Date: Aug. 23, 2024

(30) Foreign Application Priority Data
Feb. 27, 2022 (EP) 22159045
Mar. 17, 2022 (EP) 22162761
Feb. 19, 2023 (EP) 23157433

(51) Int. Cl. C07K 16/28; C07K 16/30; C07K 16/40

(52) U.S. Cl. CPC C07K 16/2827; C07K 16/28; C07K 16/30; C07K 16/40; C07K 2317/24; C07K 2317/31; C07K 2317/622; C07K 2317/73; C07K 2317/92

(57) ABSTRACT

The present invention relates to bispecific antibodies binding to CD277 and to a human tumor-antigen. The present invention relates also to polynucleotides encoding such bispecific antibodies and to vectors and host cells comprising such polynucleotides. In addition, the present invention relates to methods for producing such antibodies and to methods of using such antibodies in the treatment of diseases and their therapeutic use.

### CROSS-REFERENCE TO RELATED APPLICATIONS

The present application is a national stage application of International Patent Application No. PCT/EP2023/054762, filed on Feb. 26, 2023, which claims the benefit of and priority to European Patent Application No. EP22159045, filed on Feb. 27, 2022; European Patent Application No. EP22162761, filed on Mar. 17, 2022; and European Patent Application No. EP23157433, filed on Feb. 19, 2023, all of which are incorporated herein by reference in their entirety.

### SEQUENCE LISTING

The instant application contains a Sequence Listing which has been submitted electronically in ASCII format and is hereby incorporated by reference in its entirety. Said ASCII file, created on Feb. 17, 2023, is named “EvoPCT-seql-000001.xml” and is approximately 182,867 bytes in size.

### FIELD OF THE INVENTION

The present invention relates to bispecific antibodies binding to Butyrophilin 3 family members CD277 (BTN3A) and to a human tumor-antigen. The present invention relates also to polynucleotides encoding such bispecific antibodies and to vectors and host cells comprising such polynucleotides. In addition, the present invention relates to methods for producing such antibodies and to methods of using such antibodies in the treatment of diseases and their therapeutic use.

### BACKGROUND OF THE INVENTION

Vγ9Vδ2 T cells are the major subset of γδ T cells in peripheral blood and make about 60%-95%. Bioinformatic analyses of large meta-genomic datasets determined the relative abundance of Vγ9Vδ2 T cells within tumors and correlated this with patient outcome. Tumor-infiltrating γδ T lymphocytes (γδ TILs) were found in all tumor entities, albeit at low numbers. Importantly, a correlation between relative abundance of γδ TILs and favorable response to immune checkpoint therapy in a variety of cancers was demonstrated. (Gentles, A. J et al.; Nat. Med. 2015, 1-12; Tosolini, M.; et al.; Oncoimmunology 2017, 6, 1-10). Cancer therapies based on in vivo stimulation, or on adoptive T cell transfer of Vγ9Vδ2 T cells, have been tested in the past decades but have failed to provide consistent clinical efficacy. Further concepts such as γδ Chimeric Antigen Receptor (CAR)-T cells and γδ T-cell engagers are currently under preclinical evaluation (Kuenkele K P., et al.; Cells 2020, 9, 829).

Butyrophilin 3 family member BTN3A (CD277; UniProtKB-000481 (BT3A1_HUMAN)) is a transmembrane receptor that harbors two extracellular Immunoglobulin (Ig)-like domains and an intracellular B30.2 domain. CD277 plays a role in T-cell activation and in the adaptive immune response and regulates the proliferation of activated T-cells, regulates the release of cytokines and IFNγ by activated T-cells, mediates the response of T-cells toward infected and transformed cells that are characterized by high levels of phosphorylated metabolites, such as isopentenyl pyrophosphate (Afrache, H., et al., Immunogenetics 64, 781-794 (2012).

(E)-4-hydroxy-3-methyl-but-2-enyl pyrophosphate) HMBPP is an essential intermediate product of the prokaryotic non-mevalonate/2-C-methyl-D-erythritol 4-phosphate/1-deoxy-D-xylulose 5-phosphate (MEP) pathway for isoprenoid synthesis. BTN3A is exquisitely tuned to recognize this pathogen-derived molecule comparable to how TLRs recognize conserved pathogen structures, such as LPS or DNA (O'Neill, L. A. J.; et al.; Nat. Rev. Immunol. 2013, 13, 453-460; Gu, S. et al.; Front. Immunol. 2014, 5, 688; Vavassori, S. et al.; Nat. Immunol. 2013, 14, 908-916). The intracellular domain B30.2 of BTN3A1 interacts directly with the bacterial metabolite HMBPP (Rhodes, D. A. et al.; J. Immunol. 2015, 194, 2390-2398; Harly, C.; et al. Blood 2012, 120, 2269-2279; Sandstrom, A.; et al.; Immunity 2014, 40, 490-500). Interaction between BTN3A1 and HMBPP results in binding of BTN3A1 to components of an immunological synapse which includes the γδ TCR and in subsequent activation of Vδ2 T cells. Butyrophilin 3A1 plays an essential role in prenyl pyrophosphate stimulation of human Vγ9Vδ2 T Cells (Wang H. et al. J Immunol 2013; 191:1029-1042; Sandstrom A. et al.; Immunity Volume 40, Issue 4, 17 Apr. 2014, Pages 490-500, Janssen O. et al., J Immunol 1991; 146; 35-39).

CD277 is an indispensable compound of every tumor (Liang, F. et al., Febs Open Bio 2021 11, 2586-2599; Ghigo, C. et al., J Immunother Cancer 2020 8, A3-A3). Payne K K. et al.; Science 369, 942-949 (2020) describe that BTN3A1 governs antitumor responses by coordinating αβ and γδ T cells.

De Bruin et al. (De Bruin R C G. et al.; Oncoimmunology 2018, VOL. 7, NO. 1, e1375641) describe a bispecific nanobody approach targeting both Vγ9Vδ2 T cells and EGFR which induces Vγ9V62-T cell activation and subsequent tumor cell lysis both in vitro and in an in vivo mouse xenograft model, demonstration the cytolytic capacity of Vγ9Vδ2 T cells.

Palakodeti et al. (Palakodeti A. et al.; JBC Vol. 287, No. 39, pp. 32780-32790, 2012) describe the modulation of human Vγ9Vδ2 T cell responses by CD277-specific antibodies. WO2012080769 and WO2020025703 relate to anti-BTN3A1 antibodies and uses thereof. BTN3A1 agonists are also described in WO2012080769; WO2010106051 (US20150353643); WO2011014438; WO2017144668; WO2019211370, WO2011/014438, and WO2012080351.

WO2012080351 and WO2012080769 refer to anti-C277 antibodies (7.2 and 20.1). scFv is mentioned as possible antibody format. Agonistic anti-C277 antibodies according to the state of the art activate the cytolytic function, cytokine production and proliferation of Vγ9Vδ2 T cells. The activation of Vγ9Vδ2 T cells in the peripheral blood according to De Gassart A. et al. in Science Translational Medicine 13, (2021), (https://doi.org/10.1126/scitranslmed.abj0835) induce a transient drop in circulating Vγ9V62 T cells as a consequence not of depletion but of trafficking and margination. The relevance of depletion was for the first time recognized by the inventors.

Imbert C. and Olive D. in A. Birbrair (ed.), Tumor Microenvironment, Advances in Experimental Medicine and Biology 1273 (https://doi.org/10.1007/978-3-030-49270-0_5) and in Imbert C. et al., in Advances in Experimental Medicine and Biology, (2020), Springer, Vol. 1273, 91-104, suggest bispecific antibodies targeting both CD277 and a tumor-antigen for activation of Vγ9Vδ2 T cells. In WO2020025703 also multispecific antibodies, such as bispecific antibodies, were suggested, comprising one arm comprising a Fab or scFv including the VH and VL of an anti-CD277 antibody, as bispecific molecule mAb×mAb, mAb×Fab, Fab×F(ab′)₂ or ligand ×Fab fusion protein formats are suggested.

Bispecific antibodies are known in a large amount of various formats (e.g. reviewed by Brinkmann U. and Kontermann E.; MAbs. 2017 February-March; 9(2): 182-212; see FIG. 2 of Brinkmann and Kontermann). In 2019, more than 20 different commercialized technology platforms were available for bsAb creation and development (reviewed by Lanrijn A F et al.; Nature Reviews; https://doi.org/10.1038/s41573-019-0028-1). A bispecific antibody format is described by Coloma M. J. and Morrison S. L., Nat. Biotechnol. 15:159-163 (1997); see also Ulrich Brinkmann & Roland E. Kontermann (2017), The making of bispecific antibodies, mAbs, 9:2, 182-212, DOI: 10.1080/19420862.2016.1268307. These bispecific molecules are composed of an IgG antibody, designated the master or parent module, with scFvs of different specificities coupled to the C terminus of the heavy chain (IgG-HC-scFv, “Morrison-type bispecific antibody”; see FIG. 1).

WO2010112193 (US009382323; EP2414391B1) relates to a multispecific antibody comprising a full-length antibody specifically binding to a first antigen and consisting of two antibody heavy chains and two antibody light chains; and one or more single-chain Fv fragments binding to one or more further antigens, wherein said single-chain Fv fragments are fused to said full length antibody via a peptide connector at the C- or N-terminus of the heavy or light chain of said full length antibody.

Presti et al. (Presti, E. L. et al., Frontiers in immunology 2017, 8, 975-11) describe that γδ T cells can be redirected to the cancer cell using antibodies. This can be achieved, for instance, by using bispecific antibodies, in which one binding site recognizes a tumor-specific cell surface molecule (for example, EpCAM or HER2/neu) and the other binding site targets CD3 or the Vγ9chain of the Vγ9Vδ2 TCR; such bispecific antibodies have been demonstrated effective in preclinical models (Hoh A, et al. Liver Int (2013) 33:127-36. doi:10.1111/liv.12011; Oberg H H, et al.; Cell Immunol (2015) 296:41-9).

WO2018041827 describes an adenovirus armed with a bispecific T cell engager (BiTE) wherein one of the binding domains in the BiTE is specific to a non-TCR activating protein such as BTN3A1 and one of the binding domains is specific to a tumor-antigen, such as CEA, MUC-1, EpCAM, HER receptors HER1, HER2, HER3, HER4, PEM, A33, G250, carbohydrate antigens Ley, Lex, Leb, PSMA, TAG-72, STEAP1, CD166, CD24, CD44, E-cadherin, SPARC, ErbB2 and ErbB3. WO2012080769 relates to anti-CD277 antibodies (e.g. mAb 7.2, mAb 20.1). Antibody fragments like Fv, Fab, F(ab′)₂, Fab′, dsFv, scFv, Sc(Fv)₂ and diabodies are mentioned in general.

WO2020060406 describes an antibody comprising a first binding moiety that is able to bind human CDTd and a second binding moiety that is able to bind the Vγ9 chain of the T cell receptor on γδ T cells for use in the treatment of Chronic Lymphocytic Leukemia, Multiple Myeloma or Acute Myeloid Leukemia.

Tumor-antigens are known from various studies e.g. comparing the respective mRNA levels or protein expression levels in tumor versus normal tissues or cell lines or from studies comparing the antigen density on the surface of tumor versus normal cells (Woell, S. et al., Int. J. Cancer 134, 731-739 (2014); Herlyn, M. et al., PNAS 76, 1438-1442 (1979); Rusnak, D. W. et al., Cell Prolif 580-594 (2007); Karhemo, P.-R. et al., Frontiers in pharmacology 3, 192 (2012); Imai, K. et al., Clin Cancer Res 14, 6487-6495 (2008); Coto-Llerena, M. et al., Frontiers Oncol 10, 979 (2020); Moreaux J., Biochem Biophys Res Commun 14, 148-155 (2012); Owen, D. H. et al., J Hematol Oncol 12, 61 (2019); Wu, M. et al., Cancer Epidemiology Biomarkers Prev Publ Am Assoc Cancer Res Cosponsored Am Soc Prev Oncol 8, 775-82 (1999); Tarn, C. et al., Proc National Acad Sci 105, 8387-8392 (2008)). Claudin 18 (CLD18) molecule (UniProtKB-P56856 (CLD18 HUMAN) is an integral transmembrane protein with a molecular weight of approximately 27.9/27.72 kD. Claudins are integral membrane proteins located within the tight junctions of epithelia and endothelia. Tight junctions organize a network of interconnected strands of intramembranous particles between adjacent cells. In tight junctions, Occludin and Claudins are the most prominent transmembrane protein components. Due to their strong intercellular adhesion properties they create a primary barrier to prevent and control the paracellular transport of solutes and restrict the lateral diffusion of membrane lipids and proteins to maintain cellular polarity. Tight junction forming proteins are critically involved in organizing epithelial tissue architecture. It is assumed that such proteins may be barely accessible to antibodies in well-structured epithelia but become exposed on tumor cells. Antibodies against Claudin18 and its splice variant Claudin 18.2 are e.g. described in WO2007059997, WO2008145338, US20150374789, WO2013174403, and U.S. Pat. No. 9,770,487 (U.S. Ser. No. 10/314,890; EP2958945; IMAB362). WO2021024020 describes a combination therapy with anti-Claudin18.2 antibodies and immune checkpoint inhibitors for the treatment of cancer.

STEAP-1 (six-transmembrane epithelial antigen of the prostate-1) is a 339 amino acid cell surface protein which in normal tissues is expressed predominantly in prostate cells. STEAP-1 protein expression is maintained at high levels across various states of prostate cancer, and STEAP-1 is also highly over-expressed in other human cancers such as lung and colon. The expression profile of STEAP-1 in normal and cancer tissues suggested its potential use as a target for immunotherapy. WO 2008/052187 reports anti-STEAP-1 antibodies and immunoconjugates thereof. STEAP-1×CD3 bispecific antibodies are described in WO2014165818 and WO2017055388.

FOLR1 is expressed on epithelial tumor cells of various origins, e.g., ovarian cancer, lung cancer, breast cancer, renal cancer, colorectal cancer, endometrial cancer. 10.1517/17425247.2012.694863. Epub 2012. WO2012119077 mention antibodies against FOLR1. Bispecific antibodies that target against FOLR1 and CD3 are described in WO2016/079076 and WO2021255143.

DLL3 is selectively expressed in high grade pulmonary neuroendocrine tumors including SCLC and LCNEC. Increased expression of DLL3 was observed in SCLC and LCNEC patient-derived xenograft tumors and was also confirmed in primary tumors. See Saunders et al., Sci Translational Medicine 7(302): 302ra136 (2015). Increased expression of DLL3 has also been observed in extrapulmonary neuroendocrine cancers including prostate neuroendocrine carcinoma (Puca et al., Sci TranslMed 11(484): pii: eaav0891 (2019). While DLL3 is expressed on the surface of such tumor cells, it is not expressed in normal tissues. WO2021007371 relates to anti-DLL3 antibodies and humanized, chimeric, or bispecific antibodies are suggested. WO2019195409 mentions multispecific proteins, binding to NKG2D receptor, CD16 and a tumor-antigen.

### SUMMARY OF THE INVENTION

Agonistic anti-C277 antibodies according to the state of the art activate the cytolytic function, cytokine production and proliferation of Vγ9Vδ2 T cells. Agonistic anti-C277 antibodies according to the state of the art induce a transient drop in circulating Vγ9Vδ2 T cells which is described as a consequence not of depletion but of trafficking of Vγ9Vδ2 T cells from the circulation to tissue including cancer tissue.

The inventors however have recognized that such activation of Vγ9Vδ2 T cells in the absence of tumor cells by agonistic anti-C277 antibodies according to the state of the art induces self-elimination of Vγ9Vδ2 T cells. The inventors have recognized that a bispecific antibody specifically and agonistically binding to CD277 (further named also as “bispecific anti-CD277 antibody”) and specifically binding to a human tumor-antigen (further named also as “tumor-antigen”) with properties as described below, shows superior killing of human tumor cells bearing said tumor-antigen and high safety in regard to lysis of non-tumor cells and does not induce self-elimination of Vγ9Vδ2 T cells.

In one embodiment the invention is characterized in comprising a bispecific antibody comprising a first binding part specifically and agonistically binding to human CD277 and a second binding part specifically binding to a tumor-antigen, characterized in that said first binding part is a full-length bivalent antibody and said second binding part consists of two identical single-chain Fv antibodies specifically binding to said tumor-antigen each of said single-chain Fv antibodies is linked by a peptide linker to each C-terminus of the first binding part.

In one embodiment each of said single-chain Fv antibodies is linked by a peptide linker with its N-terminus of the variable light chain to each C-terminus of the first binding part.

In one embodiment the bispecific antibody according to the invention is characterized in comprising in the first binding part as heavy chain CDR sequences CDRH1 of SEQ ID NO:2, CDRH2 of SEQ ID NO:3, and CDRH3 of SEQ ID NO:4 and as light chain CDR sequences CDRL1 of SEQ ID NO:6, CDRL2 of SEQ ID NO:7, and CDRL3 of SEQ ID NO:8.

In one embodiment the antibody according to the invention is characterized in comprising substitution of N5S and K10N (also referred as N53S, K58N (Kabat), or N185S-K190N) in CDRH2 (SEQ ID NO:44).

In one embodiment the antibody according to the invention is characterized in comprising in addition to said CDRH2 substitution a substitution of L8V (also referred as L31V) in CDRL1 (SEQ ID NO:75). In one embodiment the antibody according to the invention is characterized in comprising in addition substitution L8V and HlR in CDRL1 (SEQ ID NO: 140).

In one embodiment the bispecific antibody according to the invention is characterized in comprising a first binding part specifically and agonistically binding to human CD277 and a second binding part specifically binding to a tumor-antigen, characterized in that said first binding part is a full-length bivalent antibody, comprising in the first binding part as heavy chain CDR sequences CDRH1 of SEQ ID NO:43, CDRH2 of SEQ ID NO:44, and CDRH3 of SEQ ID NO:45 and as light chain CDR sequences CDRL1 of SEQ ID NO:6, CDRL2 of SEQ ID NO:7, and CDRL3 of SEQ ID NO:8, and said second binding part consists of two identical single-chain Fv antibodies specifically binding to said tumor-antigen each of said single-chain Fv antibodies is linked by a peptide linker to each C-terminus of the first binding part.

In one embodiment the bispecific antibody according to the invention is characterized in that CDRH2 is of SEQ ID NO:68, SEQ ID NO:72, or SEQ ID NO:110.

In one embodiment the bispecific antibody according to the invention is characterized in that CDRL1 is of SEQ ID NO:75, SEQ ID NO:121, SEQ ID NO:133, SEQ ID NO:140 or SEQ ID NO:141.

In one embodiment the antibody according to the invention is characterized in comprising substitution ofN5S and KION in CDRH2 (SEQ ID NO:44).

In one embodiment the antibody according to the invention is characterized in comprising in addition to said CDRH2 substitution a substitution of L8V in CDRL1 (SEQ ID NO:75). In one embodiment the antibody according to the invention is characterized in comprising in addition substitution L8V and HIR in CDRL1 (SEQ ID NO: 140).

In one embodiment the first binding part of the antibody according to the invention is a human, humanized or CDR grafted antibody.

In one embodiment the invention is characterized in comprising a bispecific antibody comprising a first binding part specifically and agonistically binding to human CD277 and a second binding part specifically binding to a tumor-antigen, characterized in comprising as heavy chain CDR sequences CDRH1 of SEQ ID NO:43, CDRH2 of SEQ ID NO:44, and CDRH3 of SEQ ID NO:45 (CDRH set 1), and
b) as light chain CDR sequences a CDR set selected from the group consisting of
b1) CDRL1 of SEQ ID NO:75, CDRL2 of SEQ ID NO:76, and CDRL3 of SEQ ID NO:77,
b2) CDRL1 of SEQ ID NO:79, CDRL2 of SEQ ID NO:80, and CDRL3 of SEQ ID NO:81,
b3) CDRL1 of SEQ ID NO:83, CDRL2 of SEQ ID NO:84, and CDRL3 of SEQ ID NO:85,
b4) CDRL1 of SEQ ID NO:87, CDRL2 of SEQ ID NO:88, and CDRL3 of SEQ ID NO:89,
b5) CDRL1 of SEQ ID NO:117, CDRL2 of SEQ ID NO:118, and CDRL3 of SEQ ID NO:119,
b6) CDRL1 of SEQ ID NO:121, CDRL2 of SEQ ID NO:122, and CDRL3 of SEQ ID NO:123,
b7) CDRL1 of SEQ ID NO:125, CDRL2 of SEQ ID NO:126, and CDRL3 of SEQ ID NO:127,
b8) CDRL1 of SEQ ID NO:129, CDRL2 of SEQ ID NO:130, and CDRL3 of SEQ ID NO:131,
b9) CDRL1 of SEQ ID NO:133, CDRL2 of SEQ ID NO:134, and CDRL3 of SEQ ID NO:135,
b10) CDRL1 of SEQ ID NO:137, CDRL2 of SEQ ID NO:138, and CDRL3 of SEQ ID NO:139,
b11) CDRL1 of SEQ ID NO:133, CDRL2 of SEQ ID NO:138, and CDRL3 of SEQ ID NO:139,
b12) CDRL1 of SEQ ID NO:140, CDRL2 of SEQ ID NO:134, and CDRL3 of SEQ ID NO:135,
b13) CDRL1 of SEQ ID NO:141, CDRL2 of SEQ ID NO:134, and CDRL3 of SEQ ID NO:135,
b14) CDRL1 of SEQ ID NO:141, CDRL2 of SEQ ID NO:138, and CDRL3 of SEQ ID NO:135,
b15) CDRL1 of SEQ ID NO:151, CDRL2 of SEQ ID NO:7, and CDRL3 of SEQ ID NO:8,
b16) CDRL1 of SEQ ID NO:152, CDRL2 of SEQ ID NO:7, and CDRL3 of SEQ ID NO:8,
b17) CDRL1 of SEQ ID NO:153, CDRL2 of SEQ ID NO:7, and CDRL3 of SEQ ID NO:8,
b18) CDRL1 of SEQ ID NO:6, CDRL2 of SEQ ID NO:7, and CDRL3 of SEQ ID NO:156,
b19) CDRL1 of SEQ ID NO:6, CDRL2 of SEQ ID NO:7, and CDRL3 of SEQ ID NO:157,
b20) CDRL1 of SEQ ID NO:6, CDRL2 of SEQ ID NO:7, and CDRL3 of SEQ ID NO:158,
b21) CDRL1 of SEQ ID NO:154, CDRL2 of SEQ ID NO:7, and CDRL3 of SEQ ID NO:8,
b22) CDRL1 of SEQ ID NO:155, CDRL2 of SEQ ID NO:7, and CDRL3 of SEQ ID NO:8,
and
c) said second binding part consists of two identical single-chain Fv antibodies specifically binding to said tumor-antigen, each linked to each C-terminus of the first binding part.

In one embodiment the bispecific antibody according to the invention is characterized in that for the first binding part the variable heavy chain is of SEQ ID NO:42 and the variable light chain is selected from the group consisting of SEQ ID NO: 5, SEQ ID NO: 65, SEQ ID NO:74, SEQ ID NO:78, SEQ ID NO:82, SEQ ID NO:86.

In one embodiment the bispecific antibody according to the invention is characterized in comprising humanized versions of said variable chains.

In one embodiment the bispecific antibody according to the invention is characterized in that said tumor-antigen is selected from the group consisting of CLDN18.2 (UniProtKB-P56856-2, CLD18_HUMAN), FOLR1 (UniProtKB-P15328, FOLR1_HUMAN), STEAP1 (UniProtKB-Q9UHE8, STEAl_HUMAN), or DLL3 (UniProtKB-Q9NYJ7, DLL3_HUMAN). Further useful tumor-antigens are e.g. described in Middleburg et al., Cancers (2021) 13, 287, pp 4-6.

In one embodiment the antibody according to the invention is characterized in that the first binding part is comprising a heavy and light chain CDR combination, selected from the group consisting of the CDR combinations as shown for compounds EvB #21 to 136 in table 3, or of a variable light chain and variable heavy chain combination for compounds EvB #21 to 136 in table 3, and said second binding part consists of two identical single-chain Fv antibodies specifically binding to a tumor-antigen. In one embodiment the bispecific antibody according to the invention is characterized in being humanized.

In one embodiment the antibody according to the invention is characterized in that the second binding part comprises as light chain CDRs a CDRL1 of SEQ ID NO:11, CDRL2 of SEQ ID NO: 12, and CDRL3 of SEQ ID NO:13 and as heavy chain CDRs aCDRH1 of SEQ ID NO:15, CDRH2 of SEQ ID NO:16, and CDRH3 of SEQ ID NO:17 for FOLR1 as tumor-antigen (FOLR1 CDR set).

In one embodiment the antibody according to the invention comprises in the second binding part as CDRs a CDRL1 of SEQ ID NO:19, CDRL2 of SEQ ID NO:20, and CDRL3 of SEQ ID NO:21 and CDRH1 of SEQ ID NO:23, CDRH2 of SEQ ID NO:24, and CDRH3 of SEQ ID NO:25 for STEAP1 as tumor-antigen (STEAP1 CDR set).

In one embodiment the antibody according to the invention comprises in the second binding part as CDRs a CDRL1 of SEQ ID NO:27, CDRL2 of SEQ ID NO:28, and CDRL3 of SEQ ID NO:29 and CDRH1 of SEQ ID NO:31, CDRH2 of SEQ ID NO:32, and CDRH3 of SEQ ID NO:33 for DLL3 as tumor-antigen (DLL3 CDR set).

In one embodiment the antibody according to the invention comprises in the second binding part as CDRs a CDRL1 of SEQ ID NO:35, CDRL2 of SEQ ID NO:36, and CDRL3 of SEQ ID NO:37 and CDRH1 of SEQ ID NO:39, CDRH2 of SEQ ID NO:40, and CDRH3 of SEQ ID NO:41 for CLDN18.2 as tumor-antigen CLDN 18.2 CDR set).

In one embodiment the antibody according to the invention is characterized in comprising in the second binding part the heavy and light chain variable region combination of SEQ ID NO:10 and SEQ ID NO:14 for FOLR-1 as tumor-antigen.

In one embodiment the antibody according to the invention is characterized in comprising in the second binding part the heavy and light chain variable region combination of SEQ ID NO:18 and SEQ ID NO:22 for STEAP1 as tumor-antigen.

In one embodiment the antibody according to the invention is characterized in comprising in the second binding part the heavy and light chain variable region combination of SEQ ID NO:26 and SEQ ID NO:30 for DLL3-4as tumor-antigen.

In one embodiment the antibody according to the invention is characterized in comprising in the second binding part the heavy and light chain variable region combination of SEQ ID NO:34, SEQ ID NO:38 CLDN 18.2 as tumor-antigen.

In one embodiment the antibody according to the invention is characterized in that
a) said bispecific antibody shows for lysis of a first, tumor-antigen bearing, cell line, as compared to lysis by a reference antibody comprising as heavy chain a heavy chain of SEQ ID NO:94 and as light chain a light chain of SEQ ID NO:93 an EC50 a ratio of 0.001 to 0.2,
b) said bispecific antibody shows for lysis of a second cell line, not bearing said tumor-antigen, as compared to lysis by said reference antibody an EC50 ratio of 5 to 1000, all measured in the presence of activated Vγ9Vδ2 T lymphocytes at an E/T ratio of 5:1, in the presence of 12.5 IU/mL Interleukin-2, and in the same assay under the same conditions.

In one embodiment the bispecific antibody is in the Mab-scFv format.

In one embodiment the invention is characterized in comprising a bispecific antibody in the Mab-scFv format, comprising a first binding part specifically and agonistically binding to human CD277 and a second binding part specifically binding to a tumor-antigen, characterized in that
a) said first binding part is a full-length bivalent antibody,
b) said second binding part is specifically binding to said tumor-antigen and is comprising as heavy and light chain CDRs a CDR set, selected from the group consisting of
b1) CDRL1 of SEQ ID NO:11, CDRL2 of SEQ ID NO:12, and CDRL3 of SEQ ID NO:13 and CDRH1 of SEQ ID NO:15, CDRH2 of SEQ ID NO:16, and CDRH3 of SEQ ID NO:17 for FOLR1 as tumor-antigen (FOLR1 CDR set),
b2) CDRL1 of SEQ ID NO:19, CDRL2 of SEQ ID NO:20, and CDRL3 of SEQ ID NO:21 and CDRH1 of SEQ ID NO:23, CDRH2 of SEQ ID NO:24, and CDRH3 of SEQ ID NO:25 for STEAP1 as tumor-antigen (STEAP1 CDR set), b3) CDRL1 of SEQ ID NO:27, CDRL2 of SEQ ID NO:28, and CDRL3 of SEQ ID NO:29 and CDRH1 of SEQ ID NO:31, CDRH2 of SEQ ID NO:32, and CDRH3 of SEQ ID NO:33 for DLL3 as tumor-antigen (DLL3 CDR set), b4) CDRL1 of SEQ ID NO:35, CDRL2 of SEQ ID NO:36, and CDRL3 of SEQ ID NO:37 and CDRH1 of SEQ ID NO:39, CDRH2 of SEQ ID NO:40, and CDRH3 of SEQ ID NO:41 for CLDN18.2 as tumor-antigen CLDN 18.2 CDR set),
c) said bispecific antibody shows for lysis of a first, tumor-antigen bearing, cell line, as compared to lysis by a reference antibody comprising as heavy chain a heavy chain of SEQ ID NO:94 and as light chain a light chain of SEQ ID NO:93 an EC50 a ratio of 0.001 to 0.2,
d) said bispecific antibody shows for lysis of a second cell line, not bearing said tumor-antigen, as compared to lysis by said reference antibody an EC50 ratio of 5 to 1000, all measured in the presence of activated Vγ9Vδ2 T lymphocytes at an E/T ratio of 5:1, in the presence of 12.5 IU/mL Interleukin-2, and in the same assay under the same conditions.

In one embodiment the invention comprises a bispecific antibody in the Mab-scFv format, comprising a first binding part specifically and agonistically binding to human CD277 and a second binding part specifically binding to a tumor-antigen, characterized in that
a) said first binding part is a full-length bivalent antibody, comprising as light chain CDR sequences CDRL1 of SEQ ID NO:6, CDRL2 of SEQ ID NO:7, and CDRL3 of SEQ ID NO:8 (CDRL set 1), and
b) as heavy chain CDR sequences, the CDR sequences selected from the group consisting of:
b1) CDRH1 of SEQ ID NO:43, CDRH2 of SEQ ID NO:44, and CDRH3 of SEQ ID NO:45 (CDRH set 1),
b2) CDRH1 of SEQ ID NO:47, CDRH2 of SEQ ID NO:48, and CDRH3 of SEQ ID NO:49 (CDRH set 2),
b3) CDRH1 of SEQ ID NO:51, CDRH2 of SEQ ID NO:52, and CDRH3 of SEQ ID NO:53 (CDRH set 3),
b4) CDRH1 of SEQ ID NO:55, CDRH2 of SEQ ID NO:56, and CDRH3 of SEQ ID NO:57 (CDRH set 4),
b5) CDRH1 of SEQ ID NO:59, CDRH2 of SEQ ID NO:60, and CDRH3 of SEQ ID NO:61 (CDRH set 5),
b6) CDRH1 of SEQ ID NO:63, CDRH2 of SEQ ID NO:64, and CDRH3 of SEQ ID NO:65 (CDRH set 6),
b7) CDRH1 of SEQ ID NO:67, CDRH2 of SEQ ID NO:68, and CDRH3 of SEQ ID NO:69 (CDRH set 7),
b8) CDRH1 of SEQ ID NO:71, CDRH2 of SEQ ID NO:72, and CDRH3 of SEQ ID NO:73 (CDRH set 8),
b10) CDRH1 of SEQ ID NO:105, CDRH2 of SEQ ID NO:106, and CDRH3 of SEQ ID NO:107 (CDRH set 10),
b11) CDRH1 of SEQ ID NO:109, CDRH2 of SEQ ID NO:110, and CDRH3 of SEQ ID NO:111 (CDRH set 11),
b12) CDRH1 of SEQ ID NO:113, CDRH2 of SEQ ID NO:114, and CDRH3 of SEQ ID NO:115 (CDRH set 12),
b13) CDRH1 of SEQ ID NO:59, CDRH2 of SEQ ID NO:110, and CDRH3 of SEQ ID NO:4 (CDRH set 14)
b14) CDRH1 of SEQ ID NO:59, CDRH2 of SEQ ID NO:72, and CDRH3 of SEQ ID NO:4 (CDRH set 15)
b15) CDRH1 of SEQ ID NO:67, CDRH2 of SEQ ID NO:44, and CDRH3 of SEQ ID NO:4 (CDRH set 20)
b16) CDRH1 of SEQ ID NO:105, CDRH2 of SEQ ID NO:72, and CDRH3 of SEQ ID NO:4 (CDRH set 18)
b17) CDRH1 of SEQ ID NO:105, CDRH2 of SEQ ID NO:110, and CDRH3 of SEQ ID NO:4 (CDRH set 19), and
c) said second binding part consists of two single-chain Fv antibodies (scFv) specifically binding to said tumor-antigen.

In one embodiment the invention comprises a bispecific antibody in the Mab-scFv format, comprising a first binding part specifically and agonistically binding to human CD277 and a second binding part specifically binding to a tumor-antigen, characterized in that a) said first binding part is a full-length bivalent antibody, comprising as light chain CDR sequences CDRL1 of SEQ ID NO:121, CDRL2 of SEQ ID NO:7, and CDRL3 of SEQ ID NO:8 (CDRL set 2) and
b) as heavy chain CDR sequences, the CDR sequences selected from the group consisting of:
b1) CDRH1 of SEQ ID NO:43, CDRH2 of SEQ ID NO:44, and CDRH3 of SEQ ID NO:45 (CDRH set 1),
b2) CDRH1 of SEQ ID NO:43, CDRH2 of SEQ ID NO:72, and CDRH3 of SEQ ID NO:45 (CDRH set 21),
b3) CDRH1 of SEQ ID NO:43 CDRH2 of SEQ ID NO:110, and CDRH3 of SEQ ID NO:45 (CDRH set 22),
b4) CDRH1 of SEQ ID NO:67, CDRH2 of SEQ ID NO:44, and CDRH3 of SEQ ID NO:4 (CDRH set 20),
b5) CDRH1 of SEQ ID NO:67, CDRH2 of SEQ ID NO:68, and CDRH3 of SEQ ID NO:4 (CDRH set 7),
b6) CDRH1 of SEQ ID NO:105, CDRH2 of SEQ ID NO:72, and CDRH3 of SEQ ID NO:4 (CDRH set 18),
b7) CDRH1 of SEQ ID NO:105, CDRH2 of SEQ ID NO:110, and CDRH3 of SEQ ID NO:4 (CDRH set 19), and
c) said second binding part consists of two single-chain Fv antibodies (scFv) specifically binding to said tumor-antigen.

In one embodiment the invention comprises a bispecific antibody in the Mab-scFv format, comprising a first binding part specifically and agonistically binding to human CD277 and a second binding part specifically binding to a tumor-antigen, characterized in that
a) said first binding part is a full-length bivalent antibody, comprising as light chain CDR sequences CDRL1 of SEQ ID NO:83, CDRL2 of SEQ ID NO:84, and CDRL3 of SEQ ID NO:85 (CDRL set 3) and
b) as heavy chain CDR sequences, the CDR sequences selected from the group consisting of:
b1) CDRH set 1, b2) CDRH set 2, b3) CDRH set 3, b4) CDRH set 4, b5) CDRH set 5, b6) CDRH set 6, b7) CDRH set 7, b8) CDRH set 8, and b9) CDRH1 of SEQ ID NO:2, CDRH2 of SEQ ID NO:3, and CDRH3 of SEQ ID NO:4 (CDRH set 9), b10) CDRH set 10, b11) CDRH set 11, b12) CDRH set 12, and
c) said second binding part consists of two single-chain Fv antibodies (scFv) specifically binding to said tumor-antigen.

In one embodiment the invention comprises a bispecific antibody in the Mab-scFv format, comprising a first binding part specifically and agonistically binding to human CD277 and a second binding part specifically binding to a tumor-antigen, characterized in that a) said first binding part is a full-length bivalent antibody, comprising as light chain CDR sequences CDRL1 of SEQ ID NO:133, CDRL2 of SEQ ID NO:7, and CDRL3 of SEQ ID NO:8 (CDRL set 4) and
b) as heavy chain CDR sequences, the CDR sequences selected from the group consisting of:
b1) CDRH1 of SEQ ID NO:43, CDRH2 of SEQ ID NO:44, and CDRH3 of SEQ ID NO:45 (CDRH set 1),
b2) CDRH1 of SEQ ID NO:43, CDRH2 of SEQ ID NO:72, and CDRH3 of SEQ ID NO:45 (CDRH set 21),
b3) CDRH1 of SEQ ID NO:43, CDRH2 of SEQ ID NO:110, and CDRH3 of SEQ ID NO:45 (CDRH set 22),
b4) CDRH1 of SEQ ID NO:43, CDRH2 of SEQ ID NO:44, and CDRH3 of SEQ ID NO:4 (CDRH set 1),
b5) CDRH1 of SEQ ID NO:43, CDRH2 of SEQ ID NO:72, and CDRH3 of SEQ ID NO:4 (CDRH set 21),
b6) CDRH1 of SEQ ID NO:67, CDRH2 of SEQ ID NO:68, and CDRH3 of SEQ ID NO:45 (CDRH set 7),
b7) CDRH1 of SEQ ID NO:67, CDRH2 of SEQ ID NO:72, and CDRH3 of SEQ ID NO:45 (CDRH set 23),
b8) CDRH1 of SEQ ID NO:67, CDRH2 of SEQ ID NO:106, and CDRH3 of SEQ ID NO:45 (CDRH set 24),
b9) CDRH1 of SEQ ID NO:67, CDRH2 of SEQ ID NO:110, and CDRH3 of SEQ ID NO:4 (CDRH set 25),
b10) CDRH1 of SEQID NO:105, CDRH2 of SEQID NO:114, and CDRH3 of SEQID NO:115 (CDRH set 26),
b11) CDRH1 of SEQ ID NO:105, CDRH2 of SEQ ID NO:44, and CDRH3 of SEQ ID NO:4 (CDRH set 27)
b12) CDRH1 of SEQ ID NO:105, CDRH2 of SEQ ID NO:110, and CDRH3 of SEQ ID NO:4 (CDRH set 19)
b13) CDRH1 of SEQ ID NO:105, CDRH2 of SEQ ID NO:72, and CDRH3 of SEQ ID NO:4 (CDRH set 18), and
c) said second binding part consists of two single-chain Fv antibodies (scFv) specifically binding to said tumor-antigen.

In one embodiment the invention comprises a bispecific antibody in the Mab-scFv format, comprising a first binding part specifically and agonistically binding to human CD277 and a second binding part specifically binding to a tumor-antigen, characterized in that a) said first binding part is a full-length bivalent antibody, comprising as light chain CDR sequences CDRL1 of SEQ ID NO:75, CDRL2 of SEQ ID NO:7, and CDRL3 of SEQ ID NO:8 (CDRL set 5) and
b) as heavy chain CDR sequences, the CDR sequences selected from the group consisting of:
b1) CDRH1 of SEQ ID NO:43, CDRH2 of SEQ ID NO:44, and CDRH3 of SEQ ID NO:45 (CDRH set 1),
b2) CDRH1 of SEQ ID NO:43, CDRH2 of SEQ ID NO:72, and CDRH3 of SEQ ID NO:45 (CDRH set 21),
b3) CDRH1 of SEQ ID NO:43, CDRH2 of SEQ ID NO:110, and CDRH3 of SEQ ID NO:45 (CDRH set 22),
b4) CDRH1 of SEQ ID NO:67, CDRH2 of SEQ ID NO:44, and CDRH3 of SEQ ID NO:45 (CDRH set 20),
b5) CDRH1 of SEQ ID NO:67, CDRH2 of SEQ ID NO:68, and CDRH3 of SEQ ID NO:45 (CDRH set 7), b2) CDRH1 of SEQ ID NO:105, CDRH2 of SEQ ID NO:72, and CDRH3 of SEQ ID NO:4 (CDRH set 18),
b6) CDRH1 of SEQ ID NO:105, CDRH2 of SEQ ID NO:110, and CDRH3 of SEQ ID NO:4 (CDRH set 19), and
c) said second binding part consists of two single-chain Fv antibodies (scFv) specifically binding to said tumor-antigen.

In one embodiment the invention comprises a bispecific antibody in the Mab-scFv format, comprising a first binding part specifically and agonistically binding to human CD277 and a second binding part specifically binding to a tumor-antigen, characterized in that
a) said first binding part is a full-length bivalent antibody, comprising as light chain CDR sequences CDRL1 of SEQ ID NO:140, CDRL2 of SEQ ID NO:7, and CDRL3 of SEQ ID NO:8 (CDRL set 6) and
b) as heavy chain CDR sequences, the CDR sequences selected from the group consisting of: b1) CDRH1 of SEQ ID NO:43, CDRH2 of SEQ ID NO:44, and CDRH3 of SEQ ID NO:45 (CDRH set 1),
b2) CDRH1 of SEQ ID NO:43, CDRH2 of SEQ ID NO:72, and CDRH3 of SEQ ID NO:45 (CDRH set 21),
b3) CDRH1 of SEQ ID NO:43, CDRH2 of SEQ ID NO:110, and CDRH3 of SEQ ID NO:45 (CDRH set 22),
b1) CDRH1 of SEQ ID NO:67, CDRH2 of SEQ ID NO:44, and CDRH3 of SEQ ID NO:45 (CDRH set 20),
b2) CDRH1 of SEQ ID NO:67, CDRH2 of SEQ ID NO:72, and CDRH3 of SEQ ID NO:45 (CDRH set 23)
b3) CDRH1 of SEQ ID NO:105, CDRH2 of SEQ ID NO:72, and CDRH3 of SEQ ID NO:45 (CDRH set 18),
b4) CDRH1 of SEQ ID NO:105, CDRH2 of SEQ ID NO:110, and CDRH3 of SEQ ID NO:45 (CDRH set 19), and
c) said second binding part consists of two single-chain Fv antibodies (scFv) specifically binding to said tumor-antigen.

In one embodiment the invention comprises a bispecific antibody in the Mab-scFv format, comprising a first binding part specifically and agonistically binding to human CD277 and a second binding part specifically binding to a tumor-antigen, characterized in that
a) said first binding part is a full-length bivalent antibody, comprising as light chain CDR sequences CDRL1 of SEQ ID NO:141, CDRL2 of SEQ ID NO:138, and CDRL3 of SEQ ID NO:8 (CDRL set 7) and
b) as heavy chain CDR sequences, the CDR sequences selected from the group consisting of:
b1) CDRH1 of SEQ ID NO:43, CDRH2 of SEQ ID NO:44, and CDRH3 of SEQ ID NO:45 (CDRH set 1),
b2) CDRH1 of SEQ ID NO:43, CDRH2 of SEQ ID NO:72, and CDRH3 of SEQ ID NO:45 (CDRH set 21),
b3) CDRH1 of SEQ ID NO:43, CDRH2 of SEQ ID NO:110, and CDRH3 of SEQ ID NO:45 (CDRH set 22),
b4) CDRH1 of SEQ ID NO:105, CDRH2 of SEQ ID NO:72, and CDRH3 of SEQ ID NO:45 (CDRH set 18),
b5) CDRH1 of SEQ ID NO:105, CDRH2 of SEQ ID NO:110, and CDRH3 of SEQ ID NO:45 (CDRH set 19), and
c) said second binding part consists of two single-chain Fv antibodies (scFv) specifically binding to said tumor-antigen.

In one embodiment the invention comprises a bispecific antibody in the Mab-scFv format, comprising a first binding part specifically and agonistically binding to human CD277 and a second binding part specifically binding to a tumor-antigen, characterized in that
a) said first binding part is a full-length bivalent antibody, comprising as light chain CDR sequences CDRL1 of SEQ ID NO:141, CDRL2 of SEQ ID NO:7, and CDRL3 of SEQ ID NO:8 (CDRL set 8) and
b) as heavy chain CDR sequences, the CDR sequences selected from the group consisting of:
b1) CDRH1 of SEQ ID NO:43, CDRH2 of SEQ ID NO:44, and CDRH3 of SEQ ID NO:45 (CDRH set 1),
b2) CDRH1 of SEQ ID NO:43, CDRH2 of SEQ ID NO:72, and CDRH3 of SEQ ID NO:45 (CDRH set 21),
b3) CDRH1 of SEQ ID NO:43, CDRH2 of SEQ ID NO:110, and CDRH3 of SEQ ID NO:45 (CDRH set 22),
b4) CDRH1 of SEQ ID NO:67, CDRH2 of SEQ ID NO:44, and CDRH3 of SEQ ID NO:45 (CDRH set 20),
b5) CDRH1 of SEQ ID NO:67, CDRH2 of SEQ ID NO:72, and CDRH3 of SEQ ID NO:45 (CDRH set 23), and
b6) CDRH1 of SEQ ID NO:105, CDRH2 of SEQ ID NO:72, and CDRH3 of SEQ ID NO:45 (CDRH set 18),
b7) CDRH1 of SEQ ID NO:105, CDRH2 of SEQ ID NO:110, and CDRH3 of SEQ ID NO:45 (CDRH set 19), and
c) said second binding part consists of two single-chain Fv antibodies (scFv) specifically binding to said tumor-antigen.

In one embodiment the invention comprises a bispecific antibody, comprising a first binding part specifically and agonistically binding to human CD277 and a second binding part specifically binding to a tumor-antigen, characterized in that
a) said first binding part is a full-length bivalent antibody, comprising as light chain CDR sequences CDRL1 of SEQ ID NO:133, CDRL2 of SEQ ID NO:138, and CDRL3 of SEQ ID NO: 139 (CDR set 12), and
b) as heavy chain CDR sequences, the CDR sequences selected from the group consisting of:
b1) CDRH1 of SEQ ID NO:67, CDRH2 of SEQ ID NO:44, and CDRH3 of SEQ ID NO:45 (CDRH set 20),
b2) CDRH1 of SEQ ID NO:67, CDRH2 of SEQ ID NO:68, and CDRH3 of SEQ ID NO:45 (CDRH set 23), and
b3) CDRH1 of SEQ ID NO:105, CDRH2 of SEQ ID NO:72, and CDRH3 of SEQ ID NO:45 (CDRH set 18),
b4) CDRH1 of SEQ ID NO:105, CDRH2 of SEQ ID NO:110, and CDRH3 of SEQ ID NO:45 (CDRH set 19), and
b5) CDRH1 of SEQ ID NO:43, CDRH2 of SEQ ID NO:44, and CDRH3 of SEQ ID NO:45 (CDRH set 1),
b6) CDRH1 of SEQ ID NO:43, CDRH2 of SEQ ID NO:72, and CDRH3 of SEQ ID NO:45 (CDRH set 21),
b7) CDRH1 of SEQ ID NO:43, CDRH2 of SEQ ID NO:110, and CDRH3 of SEQ ID NO:45 (CDRH set 22),
c) said second binding part consists of two single-chain Fv antibodies (scFv) specifically binding to said tumor-antigen.

In one embodiment the invention comprises a bispecific antibody in the Mab-scFv format, comprising a first binding part specifically and agonistically binding to human CD277 and a second binding part specifically binding to a tumor-antigen, characterized in that
a) said first binding part is a full-length bivalent antibody, comprising as light chain CDR sequences CDRL1 of SEQ ID NO:87, CDRL2 of SEQ ID NO:88, and CDRL3 of SEQ ID NO:89 (CDRL set 9) and
b) as heavy chain CDR sequences, the CDR sequences selected from the group consisting of:
b1) CDRH set 1, b2) CDRH set 2, b3) CDRH set 3, b4) CDRH set 4, b5) CDRH set 5, b6) CDRH set 6, b7) CDRH set 7, b8) CDRH set 8, and b9) (CDRH set 9), b10) CDRH set 10, b11) CDRH set 11, b12) CDRH set 12, and
c) said second binding part consists of two single-chain Fv antibodies (scFv) specifically binding to said tumor-antigen.

In one embodiment the invention comprises a bispecific antibody in the Mab-scFv format, comprising a first binding part specifically and agonistically binding to human CD277 and a second binding part specifically binding to a tumor-antigen, characterized in that
a) said first binding part is a full-length bivalent antibody, comprising as light chain CDR sequences CDRL1 of SEQ ID NO:79, CDRL2 of SEQ ID NO:80, and CDRL3 of SEQ ID NO:81 (CDRL set 10) and
b) as heavy chain CDR sequences, the CDR sequences selected from the group consisting of:
b1) CDRH set 1, b2) CDRH set 2, b3) CDRH set 3, b4) CDRH set 4, b5) CDRH set 5, b6) CDRH set 6, b7) CDRH set 7, b8) CDRH set 8, and b9) CDRH set 9), b10) CDRH set 10, b11) CDRH set 11, b12) CDRH set 12, and
c) said second binding part consists of two single-chain Fv antibodies (scFv) specifically binding to said tumor-antigen.

In one embodiment the invention comprises a bispecific antibody, comprising a first binding part specifically and agonistically binding to human CD277 and a second binding part specifically binding to a tumor-antigen, characterized in that
a) said first binding part is a full-length bivalent antibody, comprising as light chain CDR sequences CDRL1 of SEQ ID NO:75, CDRL2 of SEQ ID NO:76, and CDRL3 of SEQ ID NO:77 (CDRL set 11), and
b) as heavy chain CDR sequences, the CDR sequences selected from the group consisting of:
b1) CDRH set 1, b2) CDRH set 2, b3) CDRH set 3, b4) CDRH set 4, b5) CDRH set 5, b6) CDRH set 6, b7) CDRH set 7, b8) CDRH set 8, and b9) CDRH set 9), b10) CDRH set 10, b11) CDRH set 11, b12) CDRH set 12, and
c) said second binding part consists of two single-chain Fv antibodies (scFv) specifically binding to said tumor-antigen.

In one embodiment the invention comprises a bispecific antibody according to the invention in the Mab-scFv format, characterized in that the first binding part comprises as light chain CDR sequences the CDRL1 set 1 and b) as heavy chain CDR sequences, a CDR set selected from the group consisting of:
b1) CDRH set 1, b2) CDRH set 2, b3) CDRH set 3, b4) CDRH set 4, b5) CDRH set 5, b6) CDRH set 6, b7) CDRH set 7, and b8) CDRH set 8, b10) CDRH set 10, b11) CDRH set 11, b12) CDRH set 12,
and the second binding part comprises as heavy and light chain CDRs a CDR set, selected from the group consisting of FOLR1 CDR set, STEAP1 CDR set, DLL3 CDR set, and CLDN 18.2 CDR set.

In one embodiment the invention comprises a bispecific antibody according to the invention in the Mab-scFv format, characterized in that the first binding part comprises as light chain CDR sequences the CDRL1 set 2 and as heavy chain CDR sequences, a CDR set selected from the group consisting of:
b1) CDRH set 1, b2) CDRH set 2, b3) CDRH set 3, b4) CDRH set 4, b5) CDRH set 5, b6) CDRH set 6, b7) CDRH set 7, b8) CDRH set 8, and b9) CDRH set 9, b10) CDRH set 10, b11) CDRH set 11, b12) CDRH set 12,
and the second binding part comprises as heavy and light chain CDRs a CDR set, selected from the group consisting of FOLR1 CDR set, STEAP1 CDR set, DLL3 CDR set, and CLDN 18.2 CDR set.

In one embodiment the invention comprises a bispecific antibody according to the invention in the Mab-scFv format, characterized in that the first binding part comprises as light chain CDR sequences the CDRL1 set 3 and as heavy chain CDR sequences, a CDR set selected from the group consisting of:
b1) CDRH set 1, b2) CDRH set 2, b3) CDRH set 3, b4) CDRH set 4, b5) CDRH set 5, b6) CDRH set 6, b7) CDRH set 7, and b8) CDRH set 8, and b9) CDRH set 9, b10) CDRH set 10, b11) CDRH set 11, b12) CDRH set 12,
and the second binding part comprises as heavy and light chain CDRs a CDR set, selected from the group consisting of FOLR1 CDR set, STEAP1 CDR set, DLL3 CDR set, and CLDN 18.2 CDR set.

In one embodiment the invention comprises a bispecific antibody according to the invention in the Mab-scFv format, characterized in that the first binding part comprises as light chain CDR sequences the CDRL1 set 4 and as heavy chain CDR sequences, a CDR set selected from the group consisting of:
b1) CDRH set 1, b2) CDRH set 2, b3) CDRH set 3, b4) CDRH set 4, b5) CDRH set 5, b6) CDRH set 6, b7) CDRH set 7, b8) CDRH set 8, and b9) CDRH set 9, b10) CDRH set 10, b11) CDRH set 11, b12) CDRH set 12,
and the second binding part comprises as heavy and light chain CDRs a CDR set, selected from the group consisting of FOLR1 CDR set, STEAP1 CDR set, DLL3 CDR set, and CLDN 18.2 CDR set.

In one embodiment the invention comprises a bispecific antibody according to the invention in the Mab-scFv format, characterized in that the first binding part comprises as light chain CDR sequences the CDRL1 set 5 and as heavy chain CDR sequences, a CDR set selected from the group consisting of: b1) CDRH set 1, b2) CDRH set 2, b3) CDRH set 3, b4) CDRH set 4, b5) CDRH set 5, b6) CDRH set 6, b7) CDRH set 7, b8) CDRH set 8, and b9) CDRH set 9, b10) CDRH set 10, b11) CDRH set 11, b12) CDRH set 12,
and the second binding part comprises as heavy and light chain CDRs a CDR set, selected from the group consisting of FOLR1 CDR set, STEAP1 CDR set, DLL3 CDR set, and CLDN 18.2 CDR set.

One embodiment of the invention is a bispecific antibody according to the invention in the Mab-scFv format, characterized in that the first binding part is a humanized antibody

In one embodiment the invention comprises a bispecific antibody in the Mab-scFv format, characterized in that the first binding part comprises as variable heavy chain, a variable heavy chain selected from the group consisting of SEQ ID NO:42, 46, 50, 54, 58, 62, 66, and 70, or humanized versions thereof with at least 95% sequence identity to said sequence, and as light chain sequences, a sequence selected from the group consisting of:
a) SEQ ID NO 5,
b) SEQ ID NO 74,
c) SEQ ID NO 78,
d) SEQ ID NO 82,
e) SEQ ID NO 86,
or humanized versions thereof with at least 95% sequence identity to said sequence,
and the second binding part consists of two identical single-chain Fv antibodies specifically binding to said tumor-antigen each of said single-chain Fv antibodies is linked by a peptide linker to each C-terminus of the first binding part. In one embodiment the second binding part comprises as heavy and light chain variable regions a set, selected from the group consisting of
e) SEQ ID NO:10 and SEQ ID NO:14 for FOLR-1 as tumor-antigen,
f) SEQ ID NO:18 and SEQ ID NO:22 for STEAP1 as tumor-antigen,
g) SEQ ID NO:26 and SEQ ID NO:30 for DLL3-4as tumor-antigen, and
h) SEQ ID NO:34, SEQ ID NO:38 CLDN 18.2 as tumor-antigen.

In one embodiment the invention comprises a bispecific antibody in the Mab-scFv format, characterized in that the first binding part comprises as variable heavy chain, a variable heavy chain of SEQ ID NO:1, or a humanized version thereof with at least 95% sequence identity to said sequence, and as light chain sequences, a sequence selected from the group consisting of:
a) SEQ ID NO 74, SEQ ID NO 78, SEQ ID NO 82, SEQ ID NO 86, or humanized versions thereof with at least 95% sequence identity to said sequence,
and the second binding part consists of two identical single-chain Fv antibodies specifically binding to said tumor-antigen each of said single-chain Fv antibodies is linked by a peptide linker to each C-terminus of the first binding part. In one embodiment the second binding part comprises as heavy and light chain variable regions a set, selected from the group consisting of
e) SEQ ID NO:10 and SEQ ID NO:14 for FOLR-1 as tumor-antigen,
f) SEQ ID NO:18 and SEQ ID NO:22 for STEAP1 as tumor-antigen,
g) SEQ ID NO:26 and SEQ ID NO:30 for DLL3-4as tumor-antigen, and
h) SEQ ID NO:34, SEQ ID NO:38 CLDN 18.2 as tumor-antigen.

In one embodiment the invention comprises a bispecific antibody according to the invention in the Mab-scFv format, characterized in that the first binding part comprises a variable light chain and variable heavy chain set, selected from the group as described in table 3,
and the second binding part comprises a variable light chain and variable heavy chain set, selected from the group consisting of
a) SEQ ID NO:10 and SEQ ID NO:14 for FOLR1 as tumor-antigen,
b) SEQ ID NO:18 and SEQ ID NO:22 for STEAP1 as tumor-antigen,
c) SEQ ID NO:26 and SEQ ID NO:30 for DLL3 as tumor-antigen, and
d) SEQ ID NO:34, SEQ ID NO:38 CLDN18.2 as tumor-antigen.

In one embodiment the invention is characterized in comprising a bispecific antibody in the Mab-scFv format, comprising a first binding part specifically and agonistically binding to human CD277 and a second binding part specifically binding to a tumor-antigen, characterized in that
a) said first binding part is a full-length bivalent antibody,
b) said second binding part is a single-chain Fv antibody (scFv) in the Mab-scFv format, specifically binding to said tumor-antigen, comprising as heavy and light chain variable regions a set, selected from the group consisting of
b1) SEQ ID NO:10 and SEQ ID NO: 14 for FOLR1 as tumor-antigen,
b2) SEQ ID NO:18 and SEQ ID NO:22 for STEAP1 as tumor-antigen,
b3) SEQ ID NO:26 and SEQ ID NO:30 for DLL3 as tumor-antigen, and
b4) SEQ ID NO:34, SEQ ID NO:38 CLDN18.2 as tumor-antigen,
a) said bispecific antibody shows for lysis of a first, tumor-antigen bearing, cell line, as compared to lysis by a reference antibody, comprising as heavy chain a heavy chain of SEQ ID NO:94 and as light chain a light chain of SEQ ID NO:93, an EC50 a ratio of 0.001 to 0.2,
b) said bispecific antibody shows for lysis of a second cell line, not bearing said tumor-antigen, as compared to lysis by said reference antibody an EC50 ratio of 5 to 1000, all measured in the presence of activated Vγ9Vδ2 T lymphocytes at an E/T ratio of 5:1, in the presence of 12.5 IU/mL Interleukin-2, and in the same assay under the same conditions.

In one embodiment the antibody according to the invention is characterized in that said first binding part is a CDR-grafted or humanized antibody. In one embodiment the human VH framework (FRH) is of IGHV1-46*01 (X92343) or IGHV4-34*01 (AB019439). In one embodiment the human VL framework (FRL) is of IGKV3-11*01 V-KAPPA (X01668) or of IGKV1-12*01 V-KAPPA (V01577); see IMGT repertoire. In one embodiment the human VH/VL framework combinations are ofIGHV1-46*01 and IGKV3-11*01, IGHV1-46*01 and IGKV1-12*01, IGHV4-34*01 and IGKV3-11*01, IGHV4-34*01 and IGKV1-12*01. According to the invention the framework sequence consists of four parts (FRH1-4 and FRL1-4).

In one embodiment the invention comprises a bispecific antibody in the Mab-scFv format, comprising a first binding part specifically and agonistically binding to human CD277 and a second binding part specifically binding to a tumor-antigen, characterized in that
a) said first binding part is a full-length bivalent antibody, comprising a variable light chain of the format FRLT-CDRLT-FRL2-CDRL2-FRL3-CDRL3-FRL4 wherein FRLT is of SEQ ID NO: 142, FRL2 is of SEQ ID NO:143, FRL3 is of SEQ ID NO:144 or 145, and FRL4 is of SEQ ID NO:146 and a variable heavy chain of the format FRH1-CDRH1-FRH2-CDRH2-FRH3-CDRH3-FRH4, wherein FRH1 is of SEQ ID NO:147, FRH2 is of SEQ ID NO:148, FRH3 is of SEQ ID NO:149, and FRH4 is of SEQ ID NO:150, combined with a CDRH/CDRL set selected from the sets of table 6 or 7 and
c) said second binding part consists of two single-chain Fv antibodies (scFv) specifically binding to said tumor-antigen.

In one embodiment the antibody according to the invention is characterized in being a humanized antibody, comprising a variable light chain consisting of the sequence of FRL1-CDRL1-FRL2-CDRL2-FRL3-CDRL3-FRL4 and a variable heavy chain consisting of the sequence of FRH1-CDRH1-FRH2-CDRH2-FRH3-CDRH3-FRH4 or a variable light chain consisting of the sequence of FRL1-CDRL1-FRL2-CDRL2-FRL3a-CDRL3-FRL4 and a variable heavy chain consisting of the sequence of FRH1-CDRH1-FRH2-CDRH2-FRH3-CDRH3-FRH4 and a CDRH/CDRL set selected from the sets of table 6 or 7.

In one embodiment the invention is characterized in that said second binding part consists of two identical single-chain Fv antibodies specifically binding to said tumor-antigen. In one embodiment said second binding part consists of two identical single-chain Fv antibodies (scFv;) specifically binding to said tumor-antigen, each linked by its N-terminus to each C-terminus of the first binding part. Therefore, to each C-terminus of the Fc part of the first binding part (which is a full-length monospecific anti-CD277 antibody) only one scFv is linked. The format of said bispecific antibody, consisting of a full-length bivalent antibody as first binding part and said two scFvs as second binding part is named herein as “Mab-scFv format”. An exemplary Mab-scFv format is shown in figure Ta.

In one embodiment each of said scFvs is chemically linked to each of the C-termini of the first binding part by a first peptide linker (linker1).

One embodiment the invention comprises a bispecific antibody according to the invention, characterized in that said scFvs are bound to said C-termini in the orientation peptide linker1-VL-peptide linker2-VH).

In one embodiment the peptide linker is selected from the group consisting of the peptides of SEQ ID NO:97, 98, 99, 100, and 101.

In one embodiment the invention comprises a bispecific antibody according to the invention, characterized in that said first peptide linker consists of 5-25, in one embodiment 10-25 amino acids.

One embodiment the invention comprises a bispecific antibody according to the invention, characterized in that said second peptide linker consists of 10-25 amino acids.

In one embodiment said bispecific antibody does not induce in said second cell line a significant lysis which is 10 times more, in one embodiment 5 times more, in one embodiment two times more, of background lysis.

In one embodiment of the invention said bispecific antibody shows for lysis of said first cell line an EC50 ratio in one embodiment of not more than 0.2, in one embodiment of 0.001 to 0.2, in one embodiment a ratio of 0.005 to 0.2, in one embodiment a ratio of 0.01 to 0.2, as compared to lysis by said reference antibody.

In one embodiment said bispecific antibody shows, for lysis of said second cell line, as compared to lysis by said reference antibody an EC50 ratio of 5 or more, of 10 or more, of 5 to 1000, in one embodiment 5 to 2000, in one embodiment 5 to 5000, in one embodiment 10 to 1000, in one embodiment 10 to 2000, in one embodiment 10 to 5000.

EC50 ratio according to the invention means ratio of the EC50 values as measured for cell lysis. An exemplary method is described in example 6.

In one embodiment said second, tumor-antigen negative, cell line is said first cell line wherein the tumor-antigen is inactivated (knockout cell line).

In one embodiment the invention comprises a bispecific antibody according to the invention, characterized in that said antibody induces an Emax of 0.5 or more, 0.8 or more, or 0.9 or more compared to the reference antibody. In one embodiment the invention said bispecific antibody shows for lysis of said first, tumor-antigen positive, cell line, an Emax ratio of 0.5 to 1.5, in one embodiment 0.8 to 1.5, in one embodiment 0.9 to 1.5, as compared to Emax of said reference antibody.

The reference antibody is a full-length bivalent, monospecific, and agonistic anti-CD277 antibody, comprising as heavy chain a heavy chain of SEQ ID NO:94 and as light chain a light chain of SEQ ID NO:93. The reference antibody comprises a variable heavy chain of SEQ ID NO:1 and a variable light chain a light chain of SEQ ID NO:5 and the CDRs of SEQ ID NO.2, 3, 4, 6, 7, 8.

In one embodiment the invention comprises a bispecific antibody according to the invention, characterized in that said tumor-antigen is a tumor-antigen, non-internalizing the bispecific antibody of the invention.

A further embodiment of the invention is a recombinant nucleic acid sequence encoding the bispecific antibody according to the invention.

A further embodiment of the invention is a vector comprising the recombinant nucleic acid sequence encoding the bispecific antibody according to the invention.

A further embodiment of the invention is a host cell, comprising a vector comprising the recombinant nucleic acid sequence encoding the bispecific antibody according to the invention.

In one embodiment the invention comprises a bispecific antibody according to the invention for use in the treatment of tumor diseases.

In one embodiment the invention comprises a bispecific antibody according to the invention, for use in the treatment of a tumor disease

In one embodiment the tumor disease is selected from the group consisting of colon carcinoma, ovarian cancer, lung cancer, prostate cancer, pancreatic cancer, breast cancer.

A further embodiment of the invention is a pharmaceutical composition comprising said bispecific antibody according to the invention.

In one embodiment the invention comprises a method of treating cancer, comprising administering an effective amount of a bispecific antibody according to the invention or a pharmaceutical composition comprising said bispecific antibody to a subject in need thereof.

The CD277 Mabs according to the invention and their properties are further described in tables 3 and 5. In one embodiment the CD277 Mab comprises an Fc domain composed of a first and a second subunit. In one embodiment the CD277 Mab comprises a second antigen binding domain that binds to a second antigen. In one embodiment the second binding part is a scFv molecule specific binding to a tumor-antigen and the antibody is in the Mab-scFv format.

### BRIEF DESCRIPTION OF THE FIGURES

FIG. 1a: One embodiment of the structure of bispecific antibodies according to the invention.

FIG. 1b: A bispecific antibody against a BTN3A agonist (HC and LC of SEQ ID NO:94 and 93, “second bispecific antibody”) and a tumor-antigen shows enhanced potency on tumor-antigen bearing cells compared to the monospecific BTN3A antibody of the same sequence, but is still binding to tumor-antigen negative cells with same potency as the monospecific BTN3A antibody of the same BTN3A antibody sequence (see lower panel). Such “second” bispecific antibody still causes unspecific activation of Vg9Vd2 cells in circulation and normal tissue. To the contrary, bispecific antibodies of the invention shows still strong potency on tumor-antigen positive cells but show less potency in tumor-antigen negative cells compared to the “second” bispecific antibody against a BTN3A agonist. Therefore, and unexpectedly, the bispecific antibodies of the invention show lower adverse side effects in therapy.

FIG. 2: Activity of EvB #5 on FOLR1+ and FOLR1− tumor cells. 10.000 Ovcar-3 (FOLR1+) or NCI-H1693 (FOLR1−) cells were cultured in complete medium (RPMI 1640 supplemented with 25 mM HEPES, 2 mM L-glutamine, 100 μg/mL streptomycin, 100 U/mL penicillin and 10% fetal bovine serum. After overnight adherence of tumor cells, cells were cultured with additional complete medium, with the indicated concentrations of antibodies and short-term activated Vγ9Vδ2 T cells in 10 IU/mL rIL-2 at an E/T ratio of 5:1. As a control for spontaneous lysis of tumor cells themselves, tumor cells in additional wells were cultured in medium with 12.5 IU/mL rIL-2 but without addition of Vγ9Vδ2 T cells or antibody (“SL”, spontaneous lysis control). As a control for maximum lysis, tumor cells in additional wells were cultured with short-term activated Vγ9Vδ2 T cells at an E/T ratio of 5:1 in medium with 12.5 IU/mL rIL-2 but with Triton-X detergent added to achieve maximum lysis (“Triton X 100” control). As a control for background lysis of tumor cells by Vγ9Vδ2 T cells, tumor cells in additional wells were cultured with short-term activated Vγ9Vδ2 T cells at an E/T ratio of 5:1 in medium with 12.5 IU/mL rIL-2 but without addition of antibodies (“Medium Ctrl”). Cell index (CI) was then measured every three minutes over 90 h. The lysis of tumor cells at time point tx was calculated by the formula as follows:

$$tumor cell lysis ( tx ) = ( CI ( tx ) - Medium Ctrl ( tx ) / ( Triton X 100 - Medium Ctrl ( tx ) ) * 100$$

Curve fitting was performed by using the sigmoidal dose-response function with Graphpad Prism 9 providing the best-fit value for maximum tumor cell lysis(tx) achieved with Reference antibody (Top value). % tumor cell lysis relative to maximum tumor cell lysis (“Top”) achieved by the Reference antibody was calculated by the following formula:

$$\% tumor cell lysis ( tx ) = tumor cell lysis ( tx ) / Top * 100.$$

FIG. 2a) shows % tumor cell lysis of FOLR1+ Ovcar-3 tumor cells, FIG. 2b) % tumor cell lysis of NCI-H1693 WT (FOLR1−) cells±SD at 24-hour time point. EC50 values for the different constructs are shown. The bispecific antibody according to the invention shows 50% killing of Ovcar-3 cells at a concentration of 0.12 nM. Background lysis±SD at 24-hour time point is indicated by Medium Ctrl. FIG. 2c) shows a comparison of % tumor cell lysis of Ovcar-3 cells for a bispecific antibody in the scFv format according to the invention and for a bispecific antibody in the inverse format. (EvB #1: full-length bivalent antibody of VH/VL combination of SEQ ID NO:1 and 5 linked to two scFvs of an anti-tumor-antigen antibody; EvB #8: full-length bivalent antibody of the VH/VL combination of the same anti-tumor-antigen antibody linked to two scFvs combined of SEQ ID NO:1 and 5 as VH/VL. Both formats are shown in FIG. 2d).

FIG. 3: Statistical analysis of lysis efficiency.

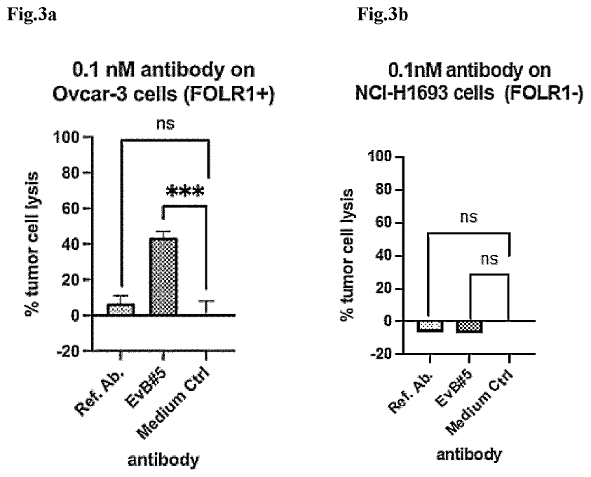

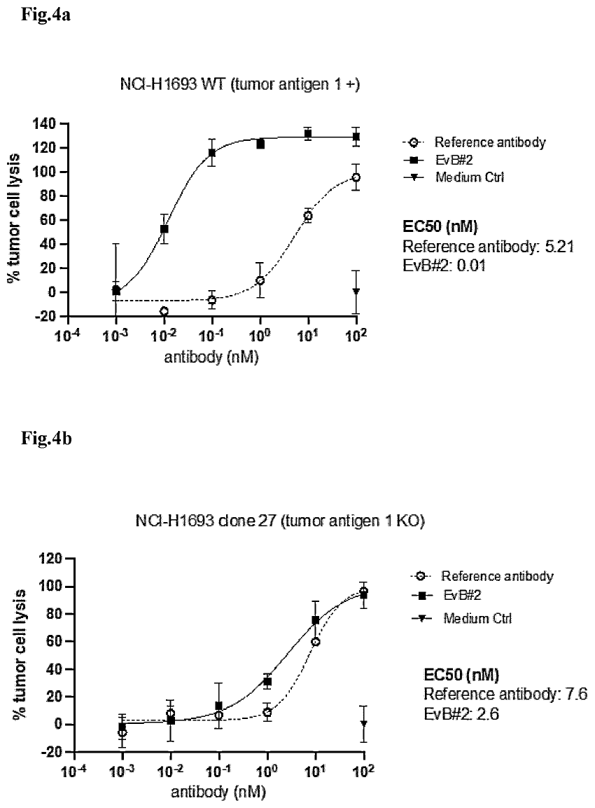

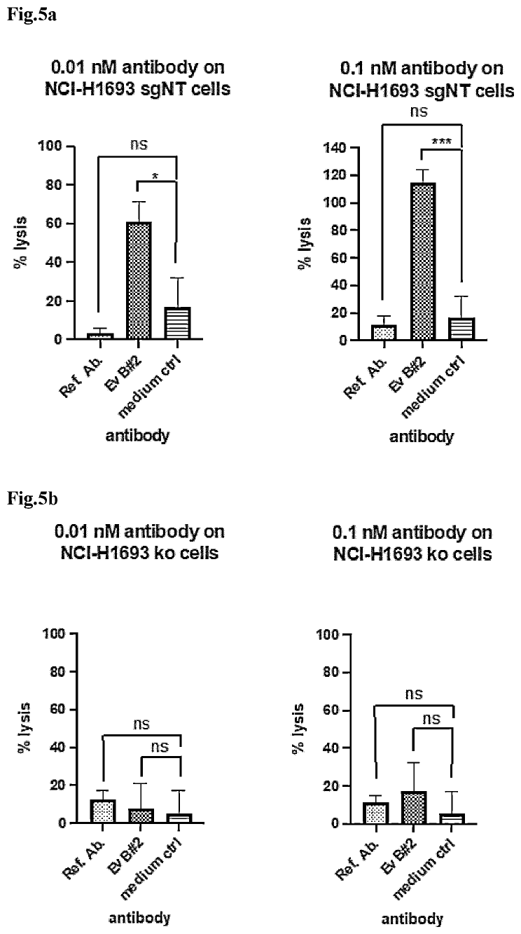

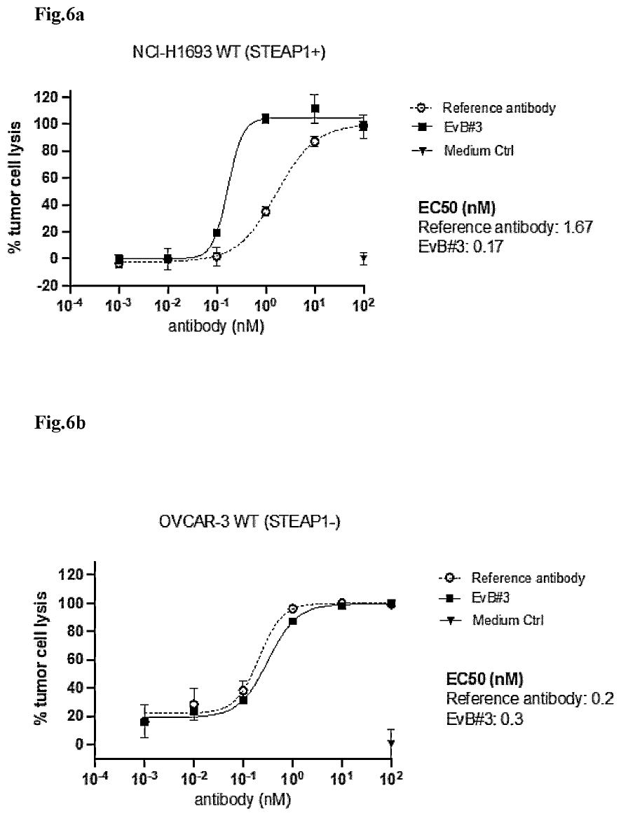

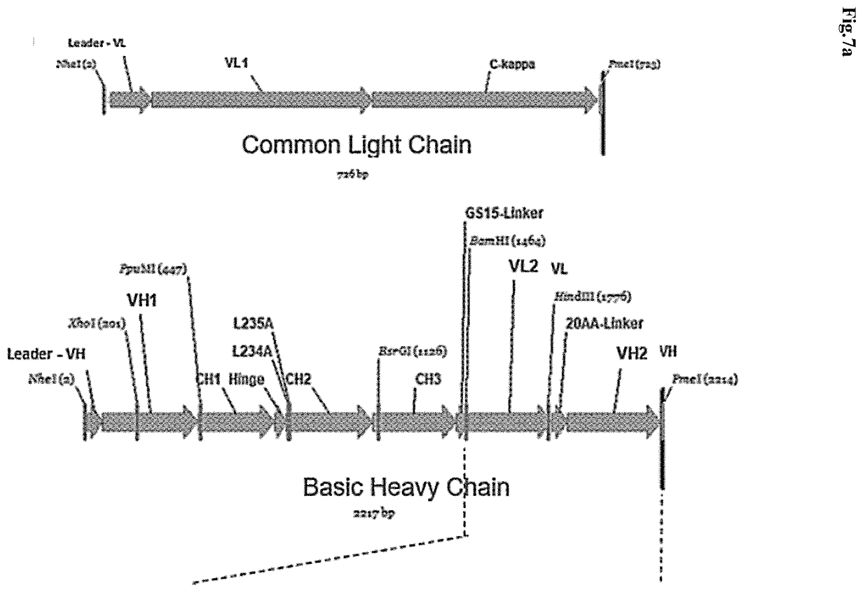

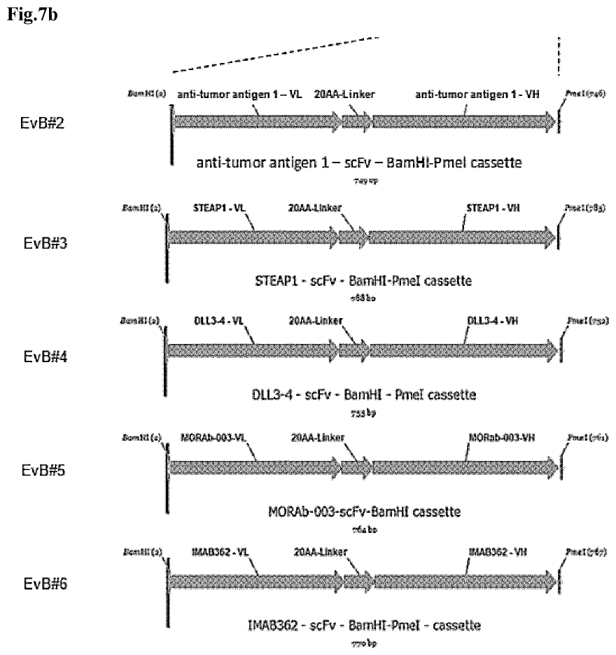

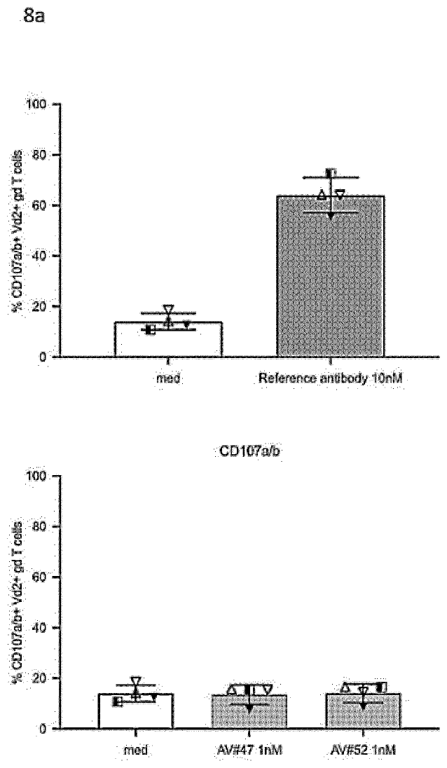

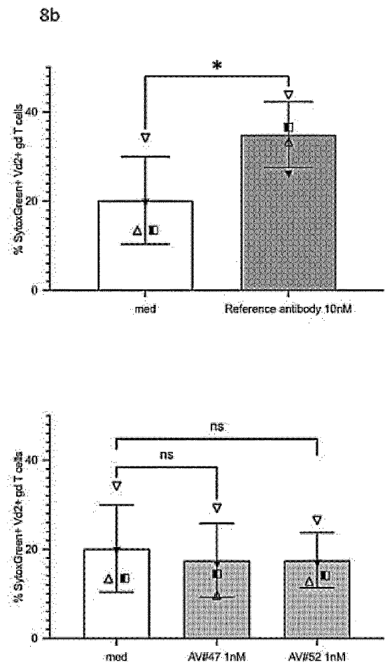

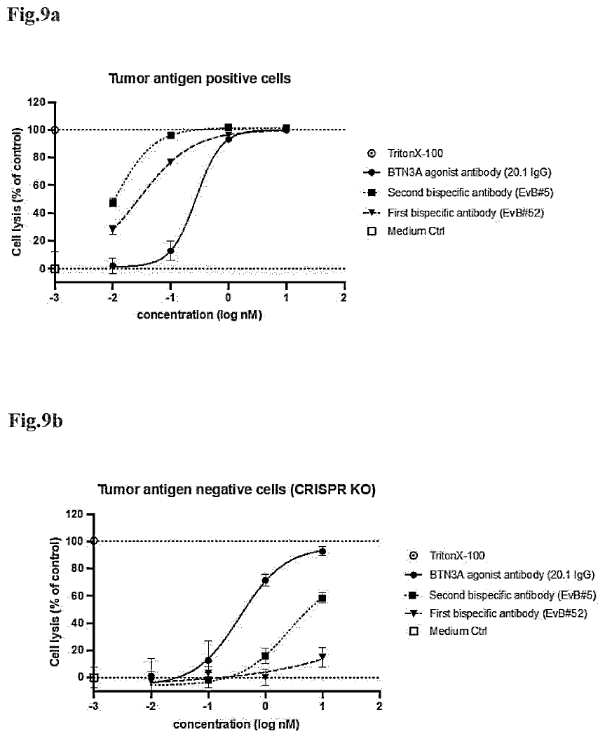

Statistical analysis demonstrates that at a concentration of 0.1 nM the bispecific antibody EvB #5 induces 48% lysis of cell line Ovcar-3, bearing FOLR1, in the presence of activated Vγ9Vδ2 T lymphocytes at an E/T ratio of 5:1 (FIG. 3a), while said bispecific antibody does not induce in NCI-H1693, not bearing FOLR1, a lysis which is significantly above background lysis (“Medium Ctrl”) in the same assay and under the same conditions (FIG. 3b). Significance of differences is determined by unpaired t-test using Graphpad Prism 9 software and the degree of significance is indicated:
ns P>0.05
* P≤0.05
** P≤0.01
*** P≤0.001
   Thus, the bispecific antibody according to the invention enhances Vγ9Vδ2 T cells cytotoxicity against FOLR1+ Ovcar-3 and not against FOLR1− NCI-H1693 cells, while the reference antibody (“Ref. Ab”) does not enhance Vγ9Vδ2 γδ T cells cytotoxicity against FOLR1+ Ovcar-3 cells.

FIG. 4. Activity of EvB #2 on NCI-H1693sgNT (WT control) and NCI-H1693sgNT with antigen 1 knockout (clone27).

10.000 NCI-H1693sgNT (WT control) or clone 27 cells were cultured in complete medium. After overnight adherence of tumor cells, cells were cultured with additional complete medium, with the indicated concentrations of antibodies and short-term activated Vγ9Vδ2 T cells in 12.5 IU/mL rIL-2 at an E/T ratio of 5:1. As a control for spontaneous lysis of tumor cells themselves, tumor cells in additional wells were cultured in medium with 12.5 IU/mL rIL-2 but without addition of Vγ9Vδ2 T cells or antibody (“SL”, spontaneous lysis control). As a control for maximum lysis, tumor cells in additional wells were cultured with short-term activated Vγ9Vδ2 T cells at an E/T ratio of 5:1 in medium with 12.5 IU/mL rIL-2 but with Triton-X detergent added to achieve maximum lysis (“Triton X 100” control). As a control for background lysis of tumor cells by Vγ9Vδ2 T cells, tumor cells in additional wells were cultured with short-term activated Vγ9Vδ2 T cells at an E/T ratio of 5:1 in medium with 12.5 IU/mL rIL-2 but without addition of antibodies (“Medium Ctrl”). Cell index (CI) was then measured every three minutes over 90 h. The lysis of tumor cells at time point tx was calculated by the formula as follows:

$$tumor cell lysis ( tx ) = ( CI ( tx ) - Medium Ctrl ( tx ) / ( Triton X 100 - Medium Ctrl ( tx ) ) * 100$$

Curve fitting was performed by using the sigmoidal dose-response function with Graphpad Prism 9 providing the best-fit value for maximum tumor cell lysis(tx) achieved with Reference antibody (Top value). % tumor cell lysis relative to maximum tumor cell lysis (“Top”) achieved by the Reference antibody was calculated by the following formula:

$$\% tumor cell lysis ( tx ) = tumor cell lysis ( tx ) / Top * 100$$

FIG. 4a) shows % tumor cell lysis of antigen 1+ NCI-H1693 sgNT tumor cells, FIG. 4b) % tumor cell lysis of antigen 1—clone 27 cells±SD at 24-hour time point. EC50 values for the different constructs are shown. The bispecific antibody according to the invention shows 50% killing of NCI-H1693 sgNT cells at a concentration of 0.012 nM. Background lysis±SD at 24-hour time point is indicated by Medium Ctrl.

FIG. 5: Statistical analysis of lysis efficiency.

Statistical analysis demonstrates that at a concentration of 0.01 nM the bispecific antibody EvB #2 induces 61% lysis of cell line NCI-H1693 sgNT, bearing antigen 1, in the presence of activated Vγ9Vδ2 T lymphocytes at an E/T ratio of 5:1 (FIG. 5a), while said bispecific antibody does not induce in NCI-H1693 ko cells (clone 27), not bearing said tumor-antigen, a lysis which is significantly above background lysis in the same assay and under the same conditions (FIG. 5b). Significance of differences is determined by unpaired t-test using Graphpad Prism 9 software and the degree of significance is indicated:
ns P>0.05
* P≤0.05
** P≤0.01
*** P≤0.001

Thus, the bispecific antibody according to the invention enhances V62+γδ T-cell cytotoxicity against NCI-H1693sgNT and not against ko cells, while the reference antibody (“Ref. Ab”) does not enhance V62+γδ T-cell cytotoxicity against tumor-antigen 1 bearing NCI-H1693sgNT cells.

FIG. 6: Activity of EvB #3 on STEAP1+ and STEAP1− tumor cells.

10.000 UMUC-3 (STEAP1+) or Ovcar-3 (STEAP1−) cells were cultured in complete medium. After overnight adherence of tumor cells, cells were cultured with additional complete medium, with the indicated concentrations of antibodies and short-term activated Vγ9Vδ2 T cells in 10 IU/mL rIL-2 at an E/T ratio of 5:1. As a control for spontaneous lysis of tumor cells themselves, tumor cells in additional wells were cultured in medium with 12.5 IU/mL rIL-2 but without addition of Vγ9Vδ2 T cells or antibody (“SL”, spontaneous lysis control). As a control for maximum lysis, tumor cells in additional wells were cultured with short-term activated Vγ9Vδ2 T cells at an E/T ratio of 5:1 in medium with 12.5 IU/mL rIL-2 but with Triton-X detergent added to achieve maximum lysis (“Triton X 100” control). As a control for background lysis of tumor cells by Vγ9Vδ2 T cells, tumor cells in additional wells were cultured with short-term activated Vγ9Vδ2 T cells at an E/T ratio of 5:1 in medium with 12.5 IU/mL rIL-2 but without addition of antibodies (“Medium Ctrl”). Cell index (CI) was then measured every three minutes over 90 h. The lysis of tumor cells at time point tx was calculated by the formula as follows:

$$tumor cell lysis ( tx ) = ( CI ( tx ) - Medium Ctrl ( tx ) / ( Triton X 100 - Medium Ctrl ( tx ) ) * 100$$

Curve fitting was performed by using the sigmoidal dose-response function with Graphpad Prism 9 providing the best-fit value for maximum tumor cell lysis(tx) achieved with Reference antibody (Top value). % tumor cell lysis relative to maximum tumor cell lysis (“Top”) achieved by the Reference antibody was calculated by the following formula:

$$\% tumor cell lysis ( tx ) = tumor cell lysis ( tx ) / Top * 100$$

FIG. 6a) shows % tumor cell lysis of STEAP1+ UMUC-3 tumor cells, FIG. 6b) % tumor cell lysis of STEAP1− Ovcar-3 tumor cells±SD at 24-hour time point (tx). EC50 values for the different constructs are shown. The bispecific antibody according to the invention shows 50% killing of UMUC-3 cells at a concentration of 0.17 nM. Background lysis±SD at 24-hour time point is indicated by Medium Ctrl.

FIG. 7: Cloning of molecules and tumor anchor cassette exchange.

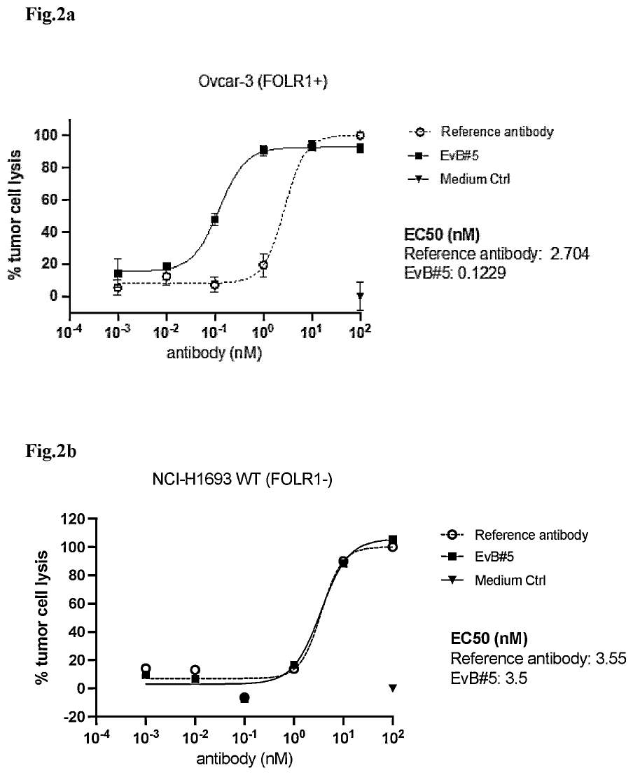

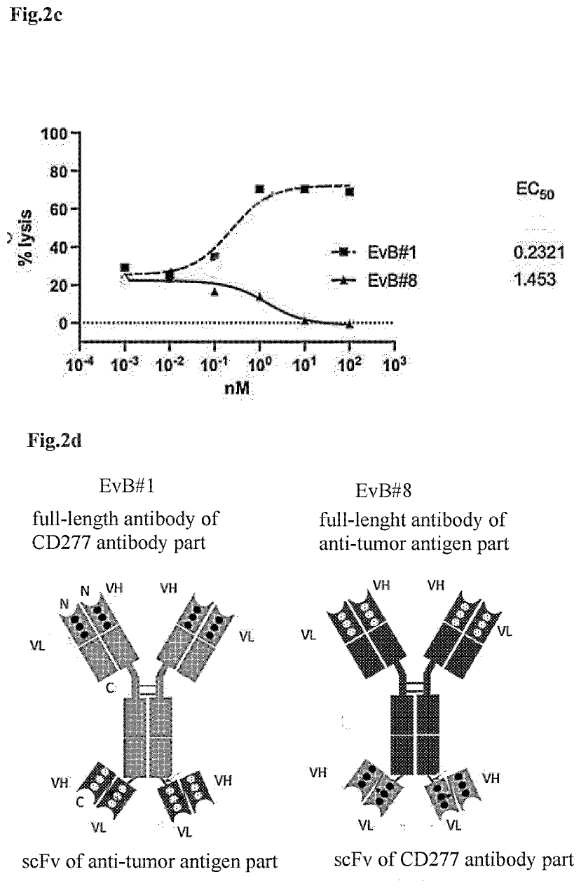

FIG. 8: shows % cell lysis of FOLR1+ Ovcar-3 and FOL1R− tumor cells (cf. FIG. 2 description). BTN3A agonist antibody: the reference antibody; EvB #5: bispecific antibody w/o CDR mutation, EvB #47 and EvB #52: bispecific antibodies with mutations, see e.g. table 2).

FIG. 9a: Vγ9Vδ2 T cell degranulation assay.

Degranulation of Vδ2 T cells in the absence of tumor-antigen positive cells was monitored by FACS analysis of CD107a levels on the cell surface. Antibodies were applied at concentrations 10fold above the efficacious concentration to reflect the higher drug levels in the primary distribution compartment after i.v. administration. The upper panel shows significant degranulation of Vγ9Vδ2 T cells from 4 different donors upon activation with reference antibody 20.1 when compared to CD107a surface levels in the presence of medium without antibody. The lower panel shows no significant degranulation of Vγ9Vδ2 T cells from 4 different donors in the presence of an antibody of the invention when compared to CD107a surface levels in the presence of medium without antibody.

FIG. 9b: Vγ9Vδ2 T cell self-elimination assay.

Self-elimination of Vδ2 T cells in the absence of tumor-antigen positive cells was monitored by FACS analysis after staining of dead cells with SytoxGreen. Antibodies were applied at concentrations 10 fold above the efficacious concentration to reflect the higher drug levels in the primary distribution compartment after i.v. administration. The upper panel shows significant killing of Vδ2 T cells from 4 different donors upon activation with reference antibody 20.1 when compared to the percentage of dead Vδ2 T cells in the presence of medium without antibody. The lower panel shows no significant killing of Vδ2 T cells from 4 different donors in the presence of an antibody of the invention.

### DETAILED DESCRIPTION OF THE INVENTION

The inventors have investigated the cell lysis for tumor-antigen positive and negative cells for an agonistic murine anti-CD277 antibody (parent antibody, 20.1) as mentioned by Imbert C. et al., WO2012080351 and WO2012080769 and for a bispecific antibody consisting of said anti-CD277 antibody and for exemplary antibodies against tumor-antigens in such bispecific antibody. As demonstrated in FIG. 9, such bispecific antibody in the Mab-scFv format shows better tumor cell lysis than the respective monospecific anti-CD277 antibody.

Surprisingly, the inventors have found in addition that a bispecific antibody of the Mab-scFv format, comprising said parent anti-CD277 antibody with two point mutations (also referred as N53S, K58N, or N5S and K10N in CDRH2 counting) in the CDR heavy chain CDRH2 (SEQ ID NO:44), provide high lysis of tumor-antigen positive cells, but reduced lysis of tumor-antigen negative cells, compared to the bispecific antibody consisting of the parent antibody without these mutations and of the anti-tumor-antigen antibody. This surprising effect is further improved by an additional point mutation (L31V, L8Vin CDRL1 counting) in the light chain CDRL1 (e.g. SEQ ID NO: 75, 140, 141) of the anti-CD277 antibody part. The invention provides therefore such bispecific antibodies and humanized versions thereof.

In one embodiment the antibody according to the invention is characterized in comprising in addition to said CDRH2 substitution a substitution of L8V in CDRL1. In one embodiment the antibody according to the invention is characterized in comprising in addition substitution L8V and HlR in CDRL1.

As used herein, the term “activated Vγ9Vδ2 T cells” according to the invention means that Vγ9Vδ2 T cells are activated by stimulation with aminobisphosphonate (n-BP) zoledronic acid and addition of recombinant IL2 (rIL2); see example 3.

The term “first binding part” refers to a full length antibody”. The term “full length antibody” as used herein refers to a heterotetrameric glycoprotein, composed of two identical light (L) chains and two identical heavy (H) chains. The full length antibody is a monospecific bivalent antibody, comprising variable and constant domains and an Fc part. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. A full length antibody consists in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3. A “full length antibody light chain” consists in N-terminal to C-terminal direction of an antibody light chain variable domain (VL), and an antibody light chain constant domain (CL), abbreviated as VL-CL. The antibody light chain constant domain (CL) can be κ (kappa) or λ (lambda). Particular amino acid residues are believed to form an interface between the light and heavy chain variable domains [Chothia et al., J. Mol. Biol., 186:651-663 (1985); Novotny and Haber, Proc. Natl. Acad. Sci. USA, 82:4592-4596 (1985)]. The light chains of antibodies from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG1, IgG2, IgG3, and IgG4; IgA1 and IgA2. The heavy chain constant domains that correspond to the different classes of immunoglobulins are called alpha, delta, epsilon, gamma, and mu, respectively.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site.

The term “humanized antibody or humanized version thereof” refers to antibodies in which the framework or “complementarity determining regions” (CDR) have been modified to comprise the CDR of an Immunoglobulin of different specificity as compared to that of the parent Immunoglobulin. In one embodiment, a murine CDR is grafted into the framework region of a human antibody to prepare the “humanized antibody or version.” See, e.g., Riechmann, L., et al., Nature 332 (1988) 323-327; and Neuberger, M. S., et al., Nature 314 (1985) 268-270. In one embodiment the human frameworks are IGHV1-46*01 (X92343) or IGHV4-34*01 (AB019439), IGKV3-11*01 V-KAPPA (X01668) or IGKV1-12*01 V-KAPPA (V01577). In one embodiment encompassed by the present invention the constant region has been additionally modified or changed from that of the original antibody to generate the properties according to the invention, especially in regard to C1q binding and/or Fc receptor (FcR) binding.

The term “variable domain” as used herein refers to an antibody region which comprises three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the R-sheet structure. The CDRs in each chain are held together in close proximity by the FR regions and, with the CDRs from the other chain, contribute to the formation of the antigen binding site of antibodies (see Kabat, E. A. et al., Sequences of Proteins of Immunological Interest, National Institutes of Health, Bethesda, MD (1987)). The constant domains are not involved directly in binding an antibody to an antigen, but exhibit various effector functions, such as participation of the antibody in antibody-dependent cellular toxicity.

The term “Fc region” as used herein refers to the C-terminal region of an immunoglobulin heavy chain. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region (using herein the numbering system according to Kabat et al., supra). The Fc region of an immunoglobulin generally comprises two constant domains, a CH2 domain and a CH3 domain, and optionally comprises a CH4 domain IgE).

By “Fc region chain” herein is meant one of the two polypeptide chains of an Fc region.

The “CH2 domain” of a human IgG Fc region (also referred to as “Cy2” domain) usually extends from an amino acid residue at about position 231 to an amino acid residue at about position 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Mol. Immunol 0.22:161-206 (1985). The CH2 domain herein may be a native sequence CH2 domain or variant CH2 domain.

The “CH3 domain” comprises the stretch of residues C-terminal to a CH2 domain in an Fc region (from an amino acid residue at about position 341 to an amino acid residue at about position 447 of an IgG). The CH3 region herein may be a native sequence CH3 domain or a variant CH3 domain (e.g. a CH3 domain with an introduced “protuberance” in one chain thereof and a corresponding introduced “cavity” in the other chain thereof; see U.S. Pat. No. 5,821,333).

“Hinge region” is generally defined as stretching from about Glu216, or about Cys226, to about Pro230 of human IgG1 (Burton, Mol. Immunol 0.22:161-206 (1985)). Hinge regions of other IgG isotypes may be aligned with the IgG1 sequence by placing the first and last cysteine residues forming inter-heavy chain S—S bonds in the same positions. The hinge region herein may be a native sequence hinge region or a variant hinge region. The two polypeptide chains of a variant hinge region generally retain at least one cysteine residue per polypeptide chain, so that the two polypeptide chains of the variant hinge region can form a disulfide bond between the two chains. The preferred hinge region herein is a native sequence human hinge region, e.g. a native sequence human IgG1 hinge region.

A “functional Fc region” possesses at least one “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

While antibodies of the IgG4 subclass show reduced Fc receptor (FcγRIIIa) binding, antibodies of other IgG subclasses show strong binding. However, Pro238, Asp265, Asp270, Asn297, Pro329, Leu234, Leu235, Gly236, Gly237, Ile253, Ser254, Lys288, Thr307, Gln311, Asn434, and His435 are residues which, if altered, provide also reduced Fe receptor binding (Shields, R. L., et al., J. Biol. Chem. 276 (2001) 6591-6604; Lund, J., et al., FASEB J. 9 (1995) 115-119; Morgan, A., et al., Immunology 86 (1995) 319-324; EP 0 307 434). In one embodiment an antibody according to the invention has a reduced FcγR binding compared to an IgG1 antibody and the full-length antibody is of IgG4 subclass or of IgG1 or IgG2 subclass with a mutation in S228, L234, L235 and/or D265, and/or contains the PVA236 mutation. In one embodiment the mutations in the full-length antibody are S228P, L234A, L235A, L235E and/or PVA236. In another embodiment the mutations in the full-length antibody are in IgG4 S228P and in IgG1 L234A and L235A.

In a further embodiment the antibody according to the invention is characterized in that said full length antibody is of human IgG1 subclass, or of human IgG1 subclass with the mutations L234A and L235A. In a further embodiment the antibody according to the invention is characterized in that said full length antibody is of human IgG4 subclass or of human IgG4 subclass with the additional mutation S228P. One embodiment comprises the mutations S228P (Ser228Pro), L235E (Leu235Glu) and P329G (Pro329Gly), or S228P (Ser228Pro), and P329G (Pro329Gly) in the constant heavy chain region of IgG4 subclass.

The term “second binding part” refers to single-chain Fv molecules. To each of the C-termini of the Fc part of the first binding part one identical single-chain Fv molecule is connected. Therefore, the second binding part comprises two single-chain Fv molecules.

As used herein, the term “single-chain Fv molecule (scFv)” refers to a molecule wherein a variable domain of a light chain (VL) is connected from its C-terminus to the N-terminal end of a variable domain of a heavy chain (VH) by a polypeptide chain. Alternately the scFv comprises of polypeptide chain where in the C-terminal end of the VH is connected to the N-terminal end of VL by a polypeptide chain.

The term “peptide linker” or “linker” as used within the invention denotes a peptide with an amino acid sequence, which is preferably of synthetic origin. The peptide linkers according to invention are used to fuse the single-chain Fab or scFv fragments to the C-terminus of the full-length antibody. Preferably said peptide linkers are peptides with an amino acid sequence with a length of at least 5 amino acids, preferably with a length of 5 to 30, more preferably of 10 to 20 amino acids. In one embodiment said peptide connector is (GxS)n or (GxS)nGm with G=glycine, S=serine, and (x=3, n=3, 4, 5 or 6, and m=0, 1, 2 or 3) or (x=4, n=2, 3, 4 or 5 and m=0, 1, 2 or 3), preferably x=4 and n=2 or 3, more preferably with x=4, n=3. In one embodiment said peptide connector is (G4S)3. Useful peptide linkers are also described in SEQ ID NOs:97-101.

The variable regions may be connected directly or, typically, via a linker peptide that allows the formation of a functional antigen binding moiety. Typical peptide linkers comprise about, and are described herein or known in the art.

The scFv molecule may be further stabilized by disulfide bridges between the heavy and light chain variable domains, for example as described in Reiter et al. (Nat. Biotechnol. 14, 1239-1245 (1996)). Hence, in one embodiment the T cell activating bi-specific antigen binding molecule of the invention comprises a scFv molecule wherein an amino acid in the heavy chain variable domain and an amino acid in the light chain variable domain have been replaced by cysteine so that a disulfide bridge can be formed between the heavy and light chain variable domain. In a specific embodiment the amino acid at position 44 of the light chain variable domain and the amino acid at position 100 of the heavy chain variable domain have been replaced by cysteine (Kabat numbering).

As is known in the art, scFvs can also be stabilized by mutation of CDR sequences, as described in (Miller et al, Protein Eng Des Sel. 2010 July; 23(7):549-57; Igawa et al, MAbs. 2011 May-June; 3(3):243-5; Perchiacca & Tessier, Annu Rev Chem Biomol Eng. 2012; 3:263-86).

In one embodiment the scFvs can be replaced by single-chain Fab fragments for improving production yield. A “single-chain Fab fragment” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single-chain Fab fragments a) VH-CH1-linker-VL-CL, b) VL-CL-linker-VH-CH1, c) VH-CL-linker-VL-CH1 and d) VL-CH1-linker-VH-CL, are stabilized via the natural disulfide bond between the CL domain and the CH1 domain. The term “N-terminus denotes the last amino acid of the N-terminus, the term “C-terminus denotes the last amino acid of the C-terminus.

The antigen binding constructs described herein are bispecific, in a general embodiment they comprise at least two antigen binding polypeptide constructs each capable of specific binding to two distinct antigens. The first binding part is a full length bivalent antibody and the second binding part consists of two monovalent antibody fragments without Fc part. In the preferred embodiment the two monovalent antibody fragments are in an scFv format, (i.e. antigen binding domains composed of a heavy chain variable domain and a light chain variable domain). In one embodiment said scFv molecules are human. In another embodiment said first and second binding part are humanized.

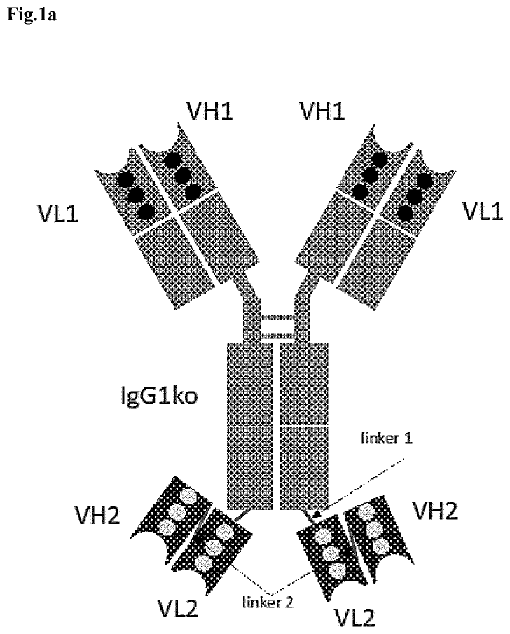

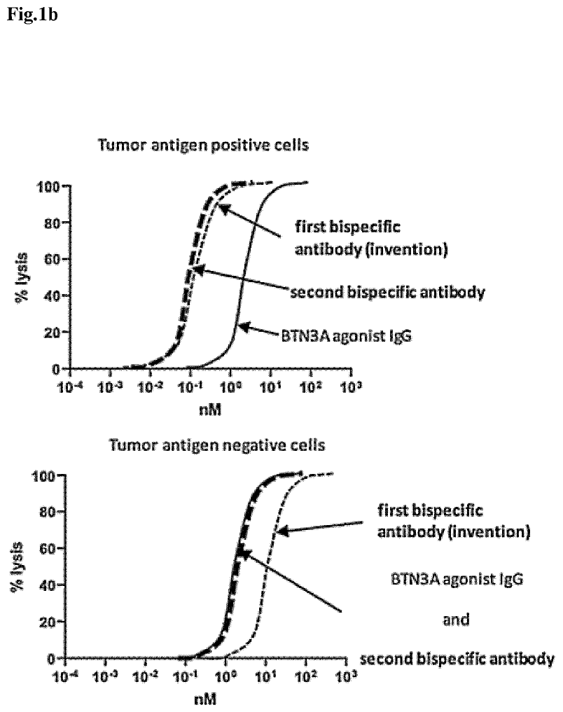

Exemplary heavy chains demonstrating the preferred Mab-scFv format (see also FIG. 1a) are shown in SEQ ID NO:102 (DLL3) and SEQ ID NO:103 (CLDN18.2).

By “specific binding” or “selective binding” is meant that the binding is selective for the antigen and can be discriminated from unwanted or non-specific interactions. The ability of an antigen binding moiety to bind to a specific antigenic determinant can be measured by surface plasmon resonance (SPR) technique (analyzed on a BIAcore instrument). In one embodiment, the extent of binding of an antigen binding moiety to an unrelated protein is less than about 10%, preferably less than 5%, of the binding of the antigen binding moiety to the antigen as measured by SPR.

The term “EC50 ratio” according to the invention means a ratio wherein the value for the bispecific antibody according of the invention is the nominator (above) and the value for the reference antibody is the denominator (below).

In one embodiment said second, tumor-antigen negative, cell line is said first cell line wherein the tumor-antigen is inactivated (knockout cell line; ko cell line).

In one embodiment the invention said bispecific antibody shows for lysis of said first, tumor-antigen positive, cell line, an Emax ratio of 0.5 to 1.5 as compared to Emax of said reference antibody. Lysis is measured by monitoring the impedance of the tumor cells (see example 6).

The term “does not induce lysis of a human cell, which does not bear said tumor-antigen” when used herein refers to lysis of tumor cells by the antibody of the invention measured in the presence of activated Vγ9Vδ2 T lymphocytes at an E/T ratio of 5:1, in the presence of 12.5 IU/mL Interleukin-2 which is not significantly different (p value>0.05) from background lysis. Background lysis is measured in the same assay under the same conditions but without addition of antibodies (“Medium Ctrl”).

As used herein “CD277 binding” means binding to BTN3A1, BTN3A2, and/or BTN3A3.

“Affinity” refers to the strength of the interactions between a single binding site of a molecule (e.g., CD277) and its binding partner (e.g., anti-CD277 antibody) represented by the dissociation constant (kD), which is the ratio of dissociation and association rate constants (koff and kon, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

As used herein “affinity matured antibody” refers to an antibody with one or more alterations in one or more CDRs thereof which result in a reduction in the affinity of the anti-CD277 antibody, compared to a parent antibody which does not possess those alteration(s). Preferred affinity matured antibodies with reduced affinity will have affinities in the nanomolar to micromolar range for CD277. Affinity matured antibodies can be produced by alanine scan (Tiller K E et al; Front. Immunol., 4 Sep. 2017 https://doi.org/10.3389/fimmu.2017.00986) or other procedures known in the art (see e.g. Tabasinezhada M. et al; Immunology Letters Volume 212, August 2019, Pages 106-113; 1.Georgiev, I. S. et al. J Immunol 192, 1100-1106 (2014).).

The terms “agonist” and “agonistic” when used herein refer to or describe a molecule which is capable of, directly or indirectly, substantially inducing, promoting, or enhancing biological activity or activation of Vγ9Vδ2 T cells (by fostering the formation of an immunological synapse to the γδ TCR). Optionally, an “agonist CD277 antibody” is an antibody which has activity that achieves the above-mentioned activation of Vγ9Vδ2 T cells by binding and activation of CD277. Preferably, the agonist is a molecule which is capable of activating human and cynomolgus Vγ9Vδ2 T cells. Even more preferably, the agonist is an antibody directed to CD277 and said antibody has agonist activity which is 5 times less potent than antibody 20.1. Agonist activity of such antibody can be determined by in an assay described in Example 6.

By “specific binding to a tumor-antigen″” is meant that the binding is selective for the tumor-antigen and can be discriminated from unwanted or non-specific interactions. The ability of a bispecific antibody according to the invention (or second binding part) to bind to a specific tumor-antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding to an unrelated protein is less than about 10% of the binding of the bispecific antibody according to the invention (or second binding part) to the tumor-antigen as measured, e.g. by SPR.

As used herein, the term “agonistic antibody specifically binding to CD277” according to the invention means that such antibody activates the cytolytic function, cytokine production and proliferation of Vγ9/Vδ2 T cells. In one embodiment, the extent of binding to an unrelated protein is less than about 10% of the binding of the bispecific antibody according to the invention (or second binding part) to the tumor-antigen as measured, e.g. by SPR In one embodiment the bispecific antibody according to the invention does not activate in a concentration of 5 nM or less, in one embodiment 20 nM or less, the cytolytic function, cytokine production and proliferation of V9/Vδ2 T cells in the absence of a tumor cell bearing said respective tumor-antigen in a cell lysis assay as described in example 6.

As used herein “tumor-antigen knock out cell line or knock out cell line” refers to a tumor cell line which bears the respective tumor-antigen in its wild-type version and wherein the respective tumor-antigen gene is inactivated. According to the invention the CRISPR/Cas9 technique may be used to introduce genetic variants of said gene and thus inactivate said antigen expression.

The term “tumor-antigen” means antigens which are presented on the surface of tumor cells, including tumor-specific antigens (TSAs) and tumor-associated antigens (TAAs). In a preferred embodiment the tumor-antigen is Claudin18.2, FOLR1, STEAP, or DLL3. Further useful tumor-antigens are e.g. described in Middleburg et al., Cancers (2021) 13, 287, pp 4-6. Some tumor-antigens like FOLR1 internalize after being bound by its natural ligand such as folate (Cheung et al., Oncotarget, 7 (32), 2016, pp 52553-32574) or therapeutic antibodies (Paulos et al., Molecular Pharmacology, 66 (6), 2004, pp 1406-1414).

Therefore, it may be that the availability in regard to recruiting of Vγ9Vδ2 T cells is diminished and a bispecific antibody according to the invention is co-internalized, which vice versa depletes the CD277-receptor on the cell surface. In such a case the receptor would not be further available for the formation of an immunological synapse contacting the Vγ9V62 T cell receptor of the immune cell. Therefore, it is preferred according to the invention that the bispecific antibody according to the invention binds to a tumor-antigen which is not internalized after binding of the respective antibody or only to such a degree that the tumor-antigen and CD277 levels remaining after (co)internalization at the cell surface are still sufficient to trigger Vg9Vd2 T cell activation.

Preferably the tumor-antigen according to the invention is selected in that both cells, antigen bearing tumor cells as well as antigen negative cells, were treated with the respective bispecific antibody or the reference antibody for 8 hours. Next, Vγ9Vδ2 T cells are added and % tumor cell lysis by surface exposed and activated CD277 is measured as described in example 6. Emax values are obtained by curve fitting and the Emax ratios for the bispecific antibody versus the reference antibody are calculated for each cell line, respectively. The Emax ratio on antigen bearing tumor cells should not be less than half of the Emax ratio on cells not bearing the tumor-antigen, indicating that the presence of the tumor-antigen did not lead to more than 50% loss of activity due to co-internalization of CD277 by the bispecific antibody on the antigen-bearing tumor cells.

As used herein, the term “Emax” refers to the response induced by any concentration of antibody or an antigen binding portion thereof, either in an in vitro or an in vivo assay, which is the maximal response.

As used herein, the term “EC50” refers to the concentration of an antibody or an antigen binding portion thereof, which induces a response in an in vitro assay, which is 50% of the maximal response, i.e., halfway between the maximal response and the baseline.

As used herein the term “KD or KD” refers to the equilibrium dissociation constant of a binding reaction between an antibody and an antigen.

The antibody according to the invention is produced by recombinant means. Thus, one embodiment of the present invention is a nucleic acid encoding the antibody according to the invention and a further embodiment is a cell comprising said nucleic acid encoding an antibody according to the invention. Methods for recombinant production are widely known in the state of the art and comprise protein expression in prokaryotic and eukaryotic cells with subsequent isolation of the antibody and usually purification to a pharmaceutically acceptable purity. For the expression of the antibodies of the invention in a host cell, nucleic acids encoding the respective modified light and heavy chains are inserted into expression vectors by standard methods. Expression is performed in appropriate prokaryotic or eukaryotic host cells like CHO cells, NS0 cells, SP2/0 cells, HEK293 cells, COS cells, PER.C6 cells, yeast, or E. coli cells, and the antibody is recovered from the cells (supernatant or cells after lysis). General methods for recombinant production of antibodies are well-known in the State of the art and described, for example, in the review articles of Makrides, S. C., Protein Expr. Purif. 17 (1999) 183-202; Geisse, S., et al., Protein Expr. Purif 8 (1996) 271-282; Kaufman, R. J., Mol. Biotechnol. 16 (2000) 151-160; Werner, R. G., Drug Res. 48 (1998) 870-880.

The bispecific antibodies according to the invention are suitably separated from the culture medium by conventional immunoglobulin purification procedures such 35 as, for example, protein A-Sepharose, hydroxylapatite chromatography, gel electrophoresis, dialysis, or affinity chromatography. DNA and RNA encoding the monoclonal antibodies is readily isolated and sequenced using conventional procedures.

The term “host cell” as used in the current application denotes any kind of cellular system which can be engineered to generate the antibodies according to the current invention. In one embodiment HEK293 cells and CHO cells are used as host cells.

One aspect of the invention is a pharmaceutical composition comprising an antibody according to the invention. Another aspect of the invention is the use of an antibody according to the invention for the manufacture of a pharmaceutical composition. A further aspect of the invention is a method for the manufacture of a pharmaceutical composition comprising an antibody according to the invention. In another aspect, the present invention provides a composition, e.g. a pharmaceutical composition, containing an antibody according to the present invention, formulated together with a pharmaceutical carrier.

One embodiment of the invention is the bispecific antibody according to the invention for use in the treatment of cancer (tumor disease).

Another aspect of the invention is said pharmaceutical composition for use the treatment of cancer.

Another aspect of the invention is the use of an antibody according to the invention for the manufacture of a medicament for the treatment of cancer.

Another aspect of the invention is method for treating of cancer in an individual, comprising administering to the individual an effective amount of a bispecific antibody according to the invention.

Another aspect of the invention is a pharmaceutical composition, comprising an antibody according to the invention.

As used herein, “pharmaceutical carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion).

A composition of the present invention can be administered by a variety of methods known in the art. As will be appreciated by the skilled artisan, the route and/or mode of administration will vary depending upon the desired results. To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

The term cancer as used herein refers to proliferative diseases, such as lymphomas, lymphocytic leukemia, lung cancer, non-small cell lung (NSCL) cancer, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, Hodgkin's Disease.

In one aspect, said cancer (tumor disease), is selected from the group consisting of colon carcinoma, ovarian cancer, lung cancer, prostate cancer, pancreatic cancer, and breast cancer.

TABLE 1
Sequence list

| Sequence NO: | Relates to | Sequence |
|---|---|---|
| SEQ ID NO: 1 | Parent VH | QVQLQQSGAELVKPGASVKLSCKASGYTFTRY |
| | | YLYWVKQRPGQGLEWIGEINPNNGGTKFNEKF |
| | | KSKATLTVDKSSRTTYIQLSSLTSEDSAVYYCS |
| | | REDDYDGTPDAMDYWGQGTAVTVSS |
| SEQ ID NO: 2 | CHRH1 | RYYLY |
| SEQ ID NO: 3 | CDRH2 | EINPNNGGTKFNEKFKS |
| SEQ ID NO: 4 | CDRH3 | EDDYDGTPDAMDY |
| SEQ ID NO: 5 | Parent VL | DIQMNQSPSSLSASLGDTITITCHASQNINLWLS |
| | | WYQQRPGNIPKLLIYRASNLHTGVPSRFSGSGS |
| | | ATGFTLTISSLQPEDIATYYCQQGHSYPYTFGG |
| | | GTKLDIK |
| SEQ ID NO: 6 | CDRL1 | HASQNINLWLS |
| SEQ ID NO: 7 | CDRL2 | RASNLHT |
| SEQ ID NO: 8 | CDRL3 | QGHSYPYT |
| SEQ ID NO: 9 | SEQ ID 12 | MGWSYIILFLVTTATGVHSQVQLQQSGAELVK |
| | EvB#5: | PGASVKLSCKASGYTFTRYYLYWVKQRPGQG |
| | Heavy chain | LEWIGEINPNNGGTKFNEKFKSKATLTVDKSSR |
| | protein (with | TTYIQLSSLTSEDSAVYYCSREDDYDGTPDAM |
| | leader): | DYWGQGTAVTVSSASTKGPSVFPLAPSSKSTSG |
| | anti-FOLR1 | GTAALGCLVKDYFPEPVTVSWNSGALTSGVHT |
| | scFv | FPAVLQSSGLYSLSSVVTVPSSSLGTQTYICNVN |
| | | HKPSNTKVDKKVEPKSCDKTHTCPPCPAPEAA |
| | | GGPSVFLFPPKPKDTLMISRTPEVTCVVVDVSH |
| | | EDPEVKFNWYVDGVEVHNAKTKPREEQYNST |
| | | YRVVSVLTVLHQDWLNGKEYKCKVSNKALPA |
| | | PIEKTISKAKGQPREPQVYTLPPSREEMTKNQV |
| | | SLTCLVKGFYPSDIAVEWESNGQPENNYKTTPP |
| | | VLDSDGSFFLYSKLTVDKSRWQQGNVFSCSVM |
| | | HEALHNHYTQKSLSLSPGKGGGGSGGGGSGGG |
| | | GSDIQLTQSPSSLSASVGDRVTITCSVSSSISSNN |
| | | LHWYQQKPGKAPKPWIYGTSNLASGVPSRFSG |
| | | SGSGTDYTFTISSLQPEDIATYYCQQWSSYPYM |
| | | YTFGQGTKVEIKGGGGSGGGGSGGGGSGGGGS |
| | | EVQLVESGGGVVQPGRSLRLSCSASGFTFSGYG |
| | | LSWVRQAPGKGLEWVAMISSGGSYTYYADSV |
| | | KGRFAISRDNAKNTLFLQMDSLRPEDTGVYFC |
| | | ARHGDDPAWFAYWGQGTPVTVSS |
| SEQ ID NO:: 10 | FOLR1-VL | DIQLTQSPSSLSASVGDRVTITCSVSSSISSNNLH |
| | | WYQQKPGKAPKPWIYGTSNLASGVPSRFSGSG |
| | | SGTDYTFTISSLQPEDIATYYCQQWSSYPYMYT |
| | | FGQGTKVEIK |
| SEQ ID NO: 11 | CDRL1 | SVSSSISSNNLH |
| SEQ ID NO: 12 | CDRL2 | GTSNLAS |
| SEQ ID NO: 13 | CDRL3 | QQWSSYPYMYT |
| SEQ ID NO: 14 | FOLR1-VH | EVQLVESGGGVVQPGRSLRLSCSASGFTFSGYG |
| | | LSWVRQAPGKGLEWVAMISSGGSYTYYADSV |
| | | KGRFAISRDNAKNTLFLQMDSLRPEDTGVYFC |
| | | ARHGDDPAWFAYWGQGTPVTVSS |
| SEQ ID NO: 15 | CDRH1 | GFTFSGYGLS |
| SEQ ID NO: 16 | CDRH2 | MISSGGSYTYYADSVKG |
| SEQ ID NO: 17 | CDRH3 | HGDDPAWFAY |
| SEQ ID NO: 18 | STEAP1-VL | DIQMTQSPSSLSASVGDRVTITCKSSQSLLYRSN |
| | | QKNYLAWYQQKPGKAPKLLIYWASTRESGVPS |
| | | RFSGSGSGTDFTLTISSLQPEDFATYYCQQYYN |
| | | YPRTFGQGTKVEIK |
| SEQ ID NO: 19 | CDRL1 | QSLLYRSNQKNYLA |
| SEQ ID NO: 20 | CDRL2 | LIYWASTRES |
| SEQ ID NO: 21 | CDRL3 | QQYYNYPR |
| SEQ ID NO: 22 | STEAP1-VH | EVQLVESGGGLVQPGGSLRLSCAVSGYSITSDY |
| | | AWNWVRQAPGKGLEWVGYISNSGSTSYNPSL |
| | | KSRFTISRDTSKNTLYLQMNSLRAEDTAVYYC |
| | | ARERNYDYDDYYYAMDYWGQGTLVTVSS |
| SEQ ID NO: 23 | CDRH1 | YSITSDYAWN |
| SEQ ID NO: 24 | CDRH2 | WVGYISNSGSTSY |
| SEQ ID NO: 25 | CDRH3 | RERNYDYDDYYYAMDY |
| SEQ ID NO: 26 | DLL3-4 VL | EIVLTQSPGTLSLSPGERVTLSCRASQRVNNNY |
| | | LAWYQQRPGQAPRLLIYGASSRATGIPDRFSGS |
| | | GSGTDFTLTISRLEPEDFAVYYCQQYDRSPLTF |
| | | GGGTKLEIK |
| SEQ ID NO: 27 | CDRL1 | RASQRVNNNYLA |
| SEQ ID NO: 28 | CDRL2 | GASSRAT |
| SEQ ID NO: 29 | CDRL3 | QQYDRSPLT |
| SEQ ID NO: 30 | DLL3-4 VH | QVQLQESGPGLVKPSETLSLTCTVSGGSISSYY |
| | | WSWIRQPPGKGLEWIGYVYYSGTTNYNPSLKS |
| | | RVTISVDTSKNQFSLKLSSVTAADTAVYYCASI |
| | | AVTGFYFDYWGQGTLVTVSS |
| SEQ ID NO: 31 | CDRH1 | SYYWS |
| SEQ ID NO: 32 | CDRH2 | YVYYSGTTNYNPSLKS |
| SEQ ID NO: 33 | CDRH3 | IAVTGFYFDY |
| SEQ ID NO: 34 | CLDN 18.2- | DIVMTQSPSSLTVTAGEKVTMSCKSSQSLLNSG |
| | VL | NQKNYLTWYQQKPGQPPKLLIYWASTRESGVP |
| | | DRFTGSGSGTDFTLTISSVQAEDLAVYYCQNDY |
| | | SYPFTFGSGTKLEIK |
| SEQ ID NO: 35 | CDRL1 | QSLLNSGNQKNYLT |
| SEQ ID NO: 36 | CDRL2 | LLIYWASTRES |
| SEQ ID NO: 37 | CDRL3 | QNDYSYPF |
| SEQ ID NO: 38 | CLDN 18.2- | QVQLQQPGAELVRPGASVKLSCKASGYTFTSY |
| | VH | WINWVKQRPGQGLEWIGNIYPSDSYTNYNQKF |
| | | KDKATLTVDKSSSTAYMQLSSPTSEDSAVYYC |
| | | TRSWRGNSFDYWGQGTTLTVSS |
| SEQ ID NO: 39 | CDRH1 | YTFTSYWIN |
| SEQ ID NO: 40 | CDRH2 | WIGNIYPSDSYTNY |
| SEQ ID NO: 41 | CDRH3 | RSWRGNSFDY |
| SEQ ID NO: 42 | VH-N185S- | QVQLQQSGAELVKPGASVKLSCKASGYTFTRY |
| | K190N | YLYWVKQRPGQGLEWIGEINPSNGGTNFNEKF |
| | | KSKATLTVDKSSRTTYIQLSSLTSEDSAVYYCSR |
| | | EDDYDGTPDAMDYWGQGTAVTVSS |
| SEQ ID NO: 43 | CDRH1 | RYYLY |
| SEQ ID NO: 44 | CDRH2 | EINPSNGGTNFNEKFKS |
| SEQ ID NO: 45 | CDRH3 | EDDYDGTPDAMDY |
| SEQ ID NO: 46 | VH-R162S- | QVQLQQSGAELVKPGASVKLSCKASGYTFTSY |
| | Y164W | WLYWVKQRPGQGLEWIGEINPNNGGTKFNEK |
| | | FKSKATLTVDKSSRTTYIQLSSLTSEDSAVYYCS |
| | | REDDYDGTPDAMDYWGQGTAVTVSS |
| SEQ ID NO: 47 | CDRH1 | SYWLY |
| SEQ ID NO: 48 | CDRH2 | EINPNNGGTKFNEKFKS |
| SEQ ID NO: 49 | CDRH3 | EDDYDGTPDAMDY |
| SEQ ID NO: 50 | VH-cluster1- | QVQLQQSGAELVKPGASVKLSCKASGYTFTSY |
| | 2: | WMHWVKQRPGQGLEWIGEINPSNGRTNYNEK |
| | | FKSKATLTVDKSSRTTYIQLSSLTSEDSAVYYCS |
| | | REDDYDGTPDAMDYWGQGTAVTVSS |
| SEQ ID NO: 51 | CDRH1 | SYWMH |
| SEQ ID NO: 52 | CDRH2 | EINPSNGRTNYNEKFKS |
| SEQ ID NO: 53 | CDRH3 | EDDYDGTPDAMDY |
| SEQ ID NO: 54 | VH-cluster1: | QVQLQQSGAELVKPGASVKLSCKASGYTFTSY |
| | | WMHWVKQRPGQGLEWIGEINPNNGGTKFNEK |
| | | FKSKATLTVDKSSRTTYIQLSSLTSEDSAVYYCS |
| | | REDDYDGTPDAMDYWGQGTAVTVSS |
| SEQ ID NO: 55 | CDRH1 | SYWMH |
| SEQ ID NO: 56 | CDRH2 | EINPNNGGTKFNEKFKS |
| SEQ ID NO: 57 | CDRH3 | EDDYDGTPDAMDY |
| SEQ ID NO: 58 | VH-cluster2 | QVQLQQSGAELVKPGASVKLSCKASGYTFTRY |
| | | YLYWVKQRPGQGLEWIGEINPSNGRTNYNEKF |
| | | KSKATLTVDKSSRTTYIQLSSLTSEDSAVYYCS |
| | | REDDYDGTPDAMDYWGQGTAVTVSS |
| SEQ ID NO: 59 | CDRH1 | RYYLY |
| SEQ ID NO: 60 | CDRH2 | EINPSNGRTNYNEKFKS |
| SEQ ID NO: 61 | CDRH3 | EDDYDGTPDAMDY |
| SEQ ID NO: 62 | VH-46_1A | QVQLVQSGAEVKKPGASVKVSCKASGYTFTRY |
| | | YLYWVRQAPGQGLEWIGEINPSNGGTNFNEKF |
| | | KSRVTLTVDKSTRTTYIELSSLRSEDTAVYYCS |
| | | REDDYDGTPDAMDYWGQGTLVTVSS |
| SEQ ID NO: 63 | CDRH1 | RYYLY |
| SEQ ID NO: 64 | CDRH2 | EINPSNGGTNFNEKFKS |
| SEQ ID NO: 65 | CDRH3 | EDDYDGTPDA MDY |
| SEQ ID NO: 66 | VH-46_1B | QVQLVQSGAE VKKPGASVKV SCKASGYTFT |
| | | RYYMYWVRQA PGQGLEWMGE INPSNGGTNF |
| | | AQKFQGRVTM TVDKSTSTVY MELSSLRSED |
| | | TAVYYCSRED DYDGTPDAMD YWGQGTLVTV |
| | | SS |
| SEQ ID NO: 67 | CDRH1 | RYYMY |
| SEQ ID NO: 68 | CDRH2 | EINPSNGGTN FAQKFQG |
| SEQ ID NO: 69 | CDRH3 | EDDYDGTPDA MDY |
| SEQ ID NO: 70 | VH-34_01A | QVQLQQSGAG LLKPSETLSL TCAAYGYTFT |
| | | RYYLYWVRQP PGKGLEWIGE INPSNGGTNF |
| | | NEKLKSRVTL SVDKSKRQTS IKLSSVTAAD |
| | | TAVYYCSRED DYDGTPDAMD YWGQGTLVTV |
| | | SS |
| SEQ ID NO: 71 | CDRH1 | RYYLY |
| SEQ ID NO: 72 | CDRH2 | EINPSNGGTN FNEKLKS |
| SEQ ID NO: 73 | CDRH3 | EDDYDGTPDA MDY |
| SEQ ID NO: 74 | Light Chain- | DIQMNQSPSSLSASLGDTITITCHASQNINVWLS |
| | L31V-R39K- | WYQQKPGNIPKLLIYKASNLHTGVPSRFSGSGS |
| | R50K-A68G- | GTGFTLTISSLQPEDIATYYCQQGQSYPLTFGGG |
| | H92Q-Y96L | TKLDIK |
| SEQ ID NO: 75 | CDRL1 | HASQNINVWLS |
| SEQ ID NO: 76 | CDRL2 | KASNLHT |
| SEQ ID NO: 77 | CDRL3 | QGQSYPLT |
| SEQ ID NO: 78 | Light Chain- | DIQMNQSPSSLSASLGDTITITCHASQNINVWLS |
| | L31V-H92Q- | WYQQRPGNIPKLLIYRASNLHTGVPSRFSGSGS |
| | Y96L | ATGFTLTISSLQPEDIATYYCQQGQSYPLTFGGG |
| | | TKLDIK |
| SEQ ID NO: 79 | CDRL1 | HASQNINVWLS |
| SEQ ID NO: 80 | CDRL2 | RASNLHT |
| SEQ ID NO: 81 | CDRL3 | QGQSYPLT |
| SEQ ID NO: 82 | Light Chain- | DIQMNQSPSSLSASLGDTITITCHASQNINLWLS |
| | H92Q-Y96L: | WYQQRPGNIPKLLIYRASNLHTGVPSRFSGSGS |
| | | ATGFTLTISSLQPEDIATYYCQQGQSYPLTFGGG |
| | | TKLDIK |
| SEQ ID NO: 83 | CDRL1 | HASQNINLWLS |
| SEQ ID NO: 84 | CDRL2 | RASNLHT |
| SEQ ID NO: 85 | CDRL3 | QGQSYPLT |
| SEQ ID NO: 86 | Light Chain- | DIQMNQSPSSLSASLGDTITITCHASQNINVWLS |
| | L31V: | WYQQRPGNIPKLLIYRASNLHTGVPSRFSGSGS |
| | | ATGFTLTISSLQPEDIATYYCQQGHSYPYTFGG |
| | | GTKLDIK |
| SEQ ID NO: 87 | CDRL1 | HASQNINVWLS |
| SEQ ID NO: 88 | CDRL2 | RASNLHT |
| SEQ ID NO: 89 | CDRL3 | QGHSYPYT |
| SEQ ID NO: 90 | IGF-1R VH | QVELVESGGGVVQPGRSQRLSCAASGFTFSSY |
| | | GMHWVRQAPGKGLEWVAIIWFDGSSTYYADS |
| | | VRGRFTISRDNSKNTLYLQMNSLRAEDTAVYF |
| | | CARELGRRYFDLWGRGTLVSVSS |
| SEQ ID NO: 91 | IGF-1R VL | EIVLTQSPATLSLSPGERATLSCRASQSVSSYLA |
| | | WYQQKPGQAPRLLIYDASKRATGIPARFSGSGS |
| | | GTDFTLTISSLEPEDFAVYYCQQRSKWPPWTFG |
| | | QGTKVESK |
| SEQ ID NO: 92 | EvB#3 | MGWSYIILFLVTTATGVHSQVQLQQSGAELVK |
| | Full length | PGASVKLSCKASGYTFTRYYLYWVKQRPGQG |
| | CD277- | LEWIGEINPNNGGTKFNEKFKSKATLTVDKSSR |
| | STEAP1 (VH) | TTYIQLSSLTSEDSAVYYCSREDDYDGTPDAM |
| | | DYWGQGTAVTVSSASTKGPSVFPLAPSSKSTSG |
| | | GTAALGCLVKDYFPEPVTVSWNSGALTSGVHT |
| | | FPAVLQSSGLYSLSSVVTVPSSSLGTQTYICNVN |
| | | HKPSNTKVDKKVEPKSCDKTHTCPPCPAPEAA |
| | | GGPSVFLFPPKPKDTLMISRTPEVTCVVVDVSH |
| | | EDPEVKFNWYVDGVEVHNAKTKPREEQYNST |
| | | YRVVSVLTVLHQDWLNGKEYKCKVSNKALPA |
| | | PIEKTISKAKGQPREPQVYTLPPSREEMTKNQV |
| | | SLTCLVKGFYPSDIAVEWESNGQPENNYKTTPP |
| | | VLDSDGSFFLYSKLTVDKSRWQQGNVFSCSVM |
| | | HEALHNHYTQKSLSLSPGKGGGGSGGGGSGGG |
| | | GSDIQMTQSPSSLSASVGDRVTITCKSSQSLLYR |
| | | SNQKNYLAWYQQKPGKAPKLLIYWASTRESG |
| | | VPSRFSGSGSGTDFTLTISSLQPEDFATYYCQQY |
| | | YNYPRTFGQGTKVEIKGGGGSGGGGSGGGGSG |
| | | GGGSEVQLVESGGGLVQPGGSLRLSCAVSGYSI |
| | | TSDYAWNWVRQAPGKGLEWVGYISNSGSTSY |
| | | NPSLKSRFTISRDTSKNTLYLQMNSLRAEDTAV |
| | | YYCARERNYDYDDYYYAMDYWGQGTLVTVS |
| | | S |
| SEQ ID NO: 93 | EvB#3 | MRVLAELLGLLLFCFLGVRCDIQMNQSPSSLSA |
| | Full length LC | SLGDTITITCHASQNINLWLSWYQQRPGNIPKL |
| | (reference LC) | LIYRASNLHTGVPSRFSGSGSATGFTLTISSLQPE |
| | | DIATYYCQQGHSYPYTFGGGTKLDIKRTVAAPS |
| | | VFIFPPSDEQLKSGTASVVCLLNNFYPREAKVQ |
| | | WKVDNALQSGNSQESVTEQDSKDSTYSLSSTL |
| | | TLSKADYEKHKVYACEVTHQGLSSPVTKSFNR |
| | | GEC |
| SEQ ID NO: 94 | Full length HC | MGWSYIILFLVTTATGVHSQVQLQQSGAELVK |
| | (reference HC) | PGASVKLSCKASGYTFTRYYLYWVKQRPGQG |
| | | LEWIGEINPNNGGTKFNEKFKSKATLTVDKSSR |
| | | TTYIQLSSLTSEDSAVYYCSREDDYDGTPDAM |
| | | DYWGQGTAVTVSSASTKGPSVFPLAPSSKSTSG |
| | | GTAALGCLVKDYFPEPVTVSWNSGALTSGVHT |
| | | FPAVLQSSGLYSLSSVVTVPSSSLGTQTYICNVN |
| | | HKPSNTKVDKKVEPKSCDKTHTCPPCPAPEAA |
| | | GGPSVFLFPPKPKDTLMISRTPEVTCVVVDVSH |
| | | EDPEVKFNWYVDGVEVHNAKTKPREEQYNST |
| | | YRVVSVLTVLHQDWLNGKEYKCKVSNKALPA |
| | | PIEKTISKAKGQPREPQVYTLPPSREEMTKNQV |
| | | SLTCLVKGFYPSDIAVEWESNGQPENNYKTTPP |
| | | VLDSDGSFFLYSKLTVDKSRWQQGNVFSCSVM |
| | | HEALHNHYTQKSLSLSPGK |
| SEQ ID NO: 95 | reference HC | MGWSYIILFLVTTATGVHS |
| | leader | |
| SEQ ID NO: 96 | RNA sequence | CAAGGAAAAGCCAGGCCCCG |
| SEQ ID NO: 97 | Linker 1 | GGGGSGGGGSGGGGS |
| SEQ ID NO: 98 | Linker 2 | GGGGSGGGGSGGGGSGGGGS |
| SEQ ID NO: 99 | Linker 3 | GSAPAPAPAPAP |
| SEQ ID | Linker 4 | APAPAPAPAP |
| NO: 100 | | |
| SEQ ID | Linker 5 | APAPAPAPAPAPAPAPAPAP |
| NO: 101 | | |
| SEQ ID | EvB#4-HC | MGWSYIILFLVTTATGVHSQVQLQQSGAELVK |
| NO: 102 | | PGASVKLSCKASGYTFTRYYLYWVKQRPGQG |
| | | LEWIGEINPNNGGTKFNEKFKSKATLTVDKSSR |
| | | TTYIQLSSLTSEDSAVYYCSREDDYDGTPDAM |
| | | DYWGQGTAVTVSSASTKGPSVFPLAPSSKSTSG |
| | | GTAALGCLVKDYFPEPVTVSWNSGALTSGVHT |
| | | FPAVLQSSGLYSLSSVVTVPSSSLGTQTYICNVN |
| | | HKPSNTKVDKKVEPKSCDKTHTCPPCPAPEAA |
| | | GGPSVFLFPPKPKDTLMISRTPEVTCVVVDVSH |
| | | EDPEVKFNWYVDGVEVHNAKTKPREEQYNST |
| | | YRVVSVLTVLHQDWLNGKEYKCKVSNKALPA |
| | | PIEKTISKAKGQPREPQVYTLPPSREEMTKNQV |
| | | SLTCLVKGFYPSDIAVEWESNGQPENNYKTTPP |
| | | VLDSDGSFFLYSKLTVDKSRWQQGNVFSCSVM |
| | | HEALHNHYTQKSLSLSPGKGGGGSGGGGSGGG |
| | | GSEIVLTQSPGTLSLSPGERVTLSCRASQRVNN |
| | | NYLAWYQQRPGQAPRLLIYGASSRATGIPDRFS |
| | | GSGSGTDFTLTISRLEPEDFAVYYCQQYDRSPL |
| | | TFGGGTKLEIKGGGGSGGGGSGGGGSGGGGSQ |
| | | VQLQESGPGLVKPSETLSLTCTVSGGSISSYYW |
| | | SWIRQPPGKGLEWIGYVYYSGTTNYNPSLKSR |
| | | VTISVDTSKNQFSLKLSSVTAADTAVYYCASIA |
| | | VTGFYFDYWGQGTLVTVSS |
| SEQ ID | EvB#6-HC | MGWSYIILFLVTTATGVHSQVQLQQSGAELVK |
| NO: 103 | | PGASVKLSCKASGYTFTRYYLYWVKQRPGQG |
| | | LEWIGEINPNNGGTKFNEKFKSKATLTVDKSSR |
| | | TTYIQLSSLTSEDSAVYYCSREDDYDGTPDAM |
| | | DYWGQGTAVTVSSASTKGPSVFPLAPSSKSTSG |
| | | GTAALGCLVKDYFPEPVTVSWNSGALTSGVHT |
| | | FPAVLQSSGLYSLSSVVTVPSSSLGTQTYICNVN |
| | | HKPSNTKVDKKVEPKSCDKTHTCPPCPAPEAA |
| | | GGPSVFLFPPKPKDTLMISRTPEVTCVVVDVSH |
| | | EDPEVKFNWYVDGVEVHNAKTKPREEQYNST |
| | | YRVVSVLTVLHQDWLNGKEYKCKVSNKALPA |
| | | PIEKTISKAKGQPREPQVYTLPPSREEMTKNQV |
| | | SLTCLVKGFYPSDIAVEWESNGQPENNYKTTPP |
| | | VLDSDGSFFLYSKLTVDKSRWQQGNVFSCSVM |
| | | HEALHNHYTQKSLSLSPGKGGGGSGGGGSGGG |
| | | GSDIVMTQSPSSLTVTAGEKVTMSCKSSQSLLN |
| | | SGNQKNYLTWYQQKPGQPPKLLIYWASTRESG |
| | | VPDRFTGSGSGTDFTLTISSVQAEDLAVYYCQN |
| | | DYSYPFTFGSGTKLEIKGGGGSGGGGSGGGGS |
| | | GGGGSQVQLQQPGAELVRPGASVKLSCKASGY |
| | | TFTSYWINWVKQRPGQGLEWIGNIYPSDSYTN |
| | | YNQKFKDKATLTVDKSSSTAYMQLSSPTSEDS |
| | | AVYYCTRSWRGNSFDYWGQGTTLTVSS |
| SEQ ID | VH-34_1B | QVQLQQSGAGLLKPSETLSLTCAAYGGTFT |
| NO: 104 | | RYYWYWVRQPPGKGLEWIGEINPSNGGTNFNE |
| | | KLKSRVTLSVDKSKRQTSIKLSSVTAADTAVYY |
| | | CSREDDYDGTPDAMDYWGQGTLVTVSS |
| SEQ ID | CDRH1 | RYYWY |
| NO: 105 | | |
| SEQ ID | CDRH2 | EINPSNGGTN FNEKLKS |
| NO: 106 | | |
| SEQ ID | CDRH3 | EDDYDGTPDAMDY |
| NO: 107 | | |
| SEQ ID | VH-34_1C | QVQLQQSGAGLLKPSETLSLTCAAYGYTFSRY |
| NO: 108 | | YLYWIRQPPGKGLEWIGEINPSNGGTNFNESLK |
| | | SRVTISVDKSKNQTSLKLSSVTAADTAVYYCSR |
| | | EDDYDGTPDAMDYWGQGTLVTVSS |
| SEQ ID | CDRH1 | RYYLY |
| NO: 109 | | |
| SEQ ID | CDRH2 | EINPSNGGTNFNESLKS |
| NO: 110 | | |
| SEQ ID | CDRH3 | EDDYDGTPDAMDY |
| NO: 111 | | |
| SEQ ID | VH-334_1D | QVQLQQSGAGLLKPSETLSLTCAAYGGTFSRY |
| NO: 112 | | YWYWIRQPPGKGLEWIGEINPSNGGTNFNESL |
| | | KSRVTISVDKSKNQTSLKLSSVTAADTAVYYCS |
| | | REDDYDGTPDAMDYWGQGTLVTVSS |
| SEQ ID | CDRH1 | RYYWY |
| NO: 113 | | |
| SEQ ID | CDRH2 | EINPSNGGTNFNESLKS |
| NO: 114 | | |
| SEQ ID | CDRH3 | EDDYDGTPDAMDY |
| NO: 115 | | |
| SEQ ID | VL-12_1A | DIQMTQSPSSVSASVGDRVTITCHASQNINLWL |
| NO: 116 | | SWYQQKPGKAPKLLIYRASNLHTGVPSRFSGS |
| | | GSATDFTLTISSLQPEDFATYYCQQGHSYPYTF |
| | | GQGTKLEIK |
| SEQ ID | CDRL1 | HASQNINLWLS |
| NO: 117 | | |
| SEQ ID | CDRL2 | RASNLHT |
| NO: 118 | | |
| SEQ ID | | |
| NO: 119 | | |
| SEQ ID | VL-12_1B | DIQMTQSPSSVSASVGDRVTITCRASQGISSWLS |
| NO: 120 | | WYQQKPGKAPKLLIYRASNLHTGVPSRFSGSG |
| | | SATDFTLTISSLQPEDFATYYCQQGHSYPYTFG |
| | | QGTKLEIK |
| SEQ ID | CDRL1 | RASQGISSWLS |
| NO: 121 | | |
| SEQ ID | CDRL2 | RASNLHT |
| NO: 122 | | |
| SEQ ID | CDRL3 | QGHSYPYT |
| NO: 123 | | |
| SEQ ID | VL-12_1C | DIQMTQSPSSVSASVGDRVTITCRASQGISSWLS |
| NO: 124 | | WYQQKPGKAPKLLIYRASNLHTGVPSRFSGSG |
| | | SGTDFTLTISSLQPEDFATYYCQQGHSYPYTFG |
| | | QGTKLEIK |
| SEQ ID | CDRL1 | RASQGISSWLS |
| NO: 125 | | |
| SEQ ID | CDRL2 | RASNLHT |
| NO: 126 | | |
| SEQ ID | CDRL3 | QGHSYPYT |
| NO: 127 | | |
| SEQ ID | VL-11_1A | EIVMTQSPATLSLSPGERATLSCHASQNINLWL |
| NO: 128 | | SWYQQKPGQAPRLLIYRASNLHTGIPARFSGSG |
| | | SATDFTLTISSLEPEDFAVYYCQQGHSYPYTFG |
| | | QGTKLEIK |
| SEQ ID | CDRL1 | HASQNINLWLS |
| NO: 129 | | |
| SEQ ID | CDRL2 | RASNLHT |
| NO: 130 | | |
| SEQ ID | CDRL3 | QGHSYPYT |
| NO: 131 | | |
| SEQ ID | VL-11_1B | EIVLTQSPATLSLSPGERATLSCRASQSVSSWLS |
| NO: 132 | | WYQQKPGQAPRLLIYRASNLHTGIPARFSGSGS |
| | | ATDFTLTISSLEPEDFAVYYCQQGHSYPYTFGQ |
| | | GTKLEIK |
| SEQ ID | CDRL1 | RASQSVSSWLS |
| NO: 133 | | |
| SEQ ID | CDRL2 | RASNLHT |
| NO: 134 | | |
| SEQ ID | CDRL3 | QGHSYPYT |
| NO: 135 | | |
| SEQ ID | VL-11_1C | EIVLTQSPATLSLSPGERATLSCRASQSVSSWLS |
| NO: 136 | | WYQQKPGQAPRLLIYRASNRHTGIPARFSGSGS |
| | | GTDFTLTISSLEPEDFAVYYCQQGHSYPYTFGQ |
| | | GTKLEIK |
| SEQ ID | CDRL1 | RASQSVSSWLS |
| NO: 137 | | |
| SEQ ID | CDRL2 | RASNRHT |
| NO: 138 | | |
| SEQ ID | CDRL3 | QGHSYPYT |
| NO: 139 | | |
| SEQ ID | CDRL1 | RASQGISVWLS |
| NO: 140 | (EvB#52) | |
| SEQ ID | CDRLla | RASQSVSVWLS |
| NO: 141 | (EvB#52) | |
| SEQ ID | FRL1 | DIQMTQSPSS VSASVGDRVTITC |
| NO: 142 | | |
| SEQ ID | FRL2 | WYQQKPGKAP KLLIY |
| NO: 143 | | |
| SEQ ID | FRL3 | GVPSRFSGSGSGTDFTLTISSLQPEDFATYYCQ |
| NO: 144 | | |
| SEQ ID | FRL3a | GIPARFSGSGSATDFTLTISSLEPEDFAVYYCQ |
| NO: 145 | | |
| SEQ ID | FRL4 | FGQGTKLEIK |
| NO: 146 | | |
| SEQ ID | FRH1 | QVQLVQSGAEVKKPGASVKVSCKASGYTFT |
| NO: 147 | | |
| SEQ ID | FRH2 | WVRQAPGQGLEWIG |
| NO: 148 | | |
| SEQ ID | FRH3 | RVTISVDKSKNQTSLKLSSVTAADTAVYYCSR |
| NO: 149 | | |
| SEQ ID | FRH4 | WGQGTLVTVSS |
| NO: 150 | | |

In the sequence list after each VH or VL the respective CDRs are listed. Several VHs and VLs comprise one or more identical CDRs:
SEQ ID NO:2 identical to SEQ ID Nos: 43, 59, 63, 71, 109.
SEQ ID NO:4 identical to SEQ ID Nos: 45, 49, 53, 57, 61, 65, 69, 73, 107, 111, 115
SEQ ID NO:7 identical to SEQ ID Nos: 80, 84, 88, 118, 122, 126, 130, 134,
SEQ ID NO:8 identical to SEQ ID Nos: 119, 89, 123, 127, 131,135, 139
SEQ ID NO:44 identical to SEQ ID No:64
SEQ ID NO:47 single SEQ
SEQ ID NO:51 identical to SEQ ID No: 54
SEQ ID NO:67 single SEQ
SEQ ID NO:75 identical to SEQ ID Nos:79, 87
SEQ ID NO:105 identical to SEQ ID No:113
SEQ ID NO:138 single SEQ

### Material and Methods

Cell culture, transfection, antibody production and purification: CHO-S cells (FreeStyle™, Thermo Fisher Scientific) were kept in Corning® Erlenmeyer flasks (125 ml, Corning, Inc.) at 37° C. and 5% CO₂ under constant shaking (120 rpm). The cells were kept in CD CHO growth medium (Gibco®/Thermo Fisher Scientific) supplemented with 1% (v/v) GlutaMAX 100× and 1% (v/v) HT Supplement 100× (Thermo Fisher Scientific). Every other day, cells were adjusted to a density of 0.3×10⁶ cells/ml to maintain exponential growth. One day prior to transfection, cells were seeded at a density of 2×10⁶ cells/ml to reach the desired density of 4×10⁶ cells/ml on the day of transfection. The transfection was performed with the MaxCyte STX™ transfection unit (MaxCyte, Inc., Gaithersburg, MD, USA) according to the manufacturer's instructions. The MaxCyte processing assembly OC-400 and an optimized transfection protocol for protein production in CHO-S cells were used. A total amount of 8×10⁸ cells were harvested by centrifugation and split to have ten equally sized portions of 8×10⁷ cells. After washing twice with 4 ml electroporation (EP) buffer, cells were resuspended in EP buffer to obtain a density of 8×10⁷ cells/400 μl. A total of 300 μg/ml plasmid DNA was added with a heavy: light chain ratio of 1:1 or a single vector for expression of surface antigens. After electroporation, CHO-S cells were directly seeded in culture flask without adding any extra buffer or media and incubated at 37° C. and 5% CO₂ for 30 minutes. Culture conditions after transfection differed between protein production and transient expression of surface markers:

Surface receptor expression: cells were kept in CD CHO growth medium for 48 h and then used for FACS analysis.

Antibody production: 150 ml production medium was added (CD OptiCHO™+1% (v/v) GlutaMAX 100×+1% (v/v) HT Supplement 100×+1% (v/v) Pluronic™ F-68 100×, all Gibco®/Thermo Fisher Scientific). One-day post transfection, 1 mM sodium butyrate (Thermo Fisher Scientific) was added, and cells were fed with 3.5% (v/v) MaxCyte Feed Stock (28 ml Yeastolate Stock Solution 0.5%+140 ml CHO CD Efficient Feed A Stock Solution+7 ml GlutaMAX 100×+24.8 ml Glucose (450 g/l) Stock Solution, Gibco®/Thermo Fisher Scientific). The incubation temperature was lowered to 32° C. for the remaining production cycle (14 days or until the cell viability dropped below 50%). During the production phase, cell density and viability were measured every other day, and cells were fed daily with MaxCyte Feed Stock (see above) until the production was stopped. Supernatants were harvested by centrifugation and filtrated to remove cellular debris (final volume about 200 ml). In a first step, affinity chromatography with the CaptureSelect™ CH1-XL (Hu) Affinity Matrix (Thermo Fisher Scientific) was performed. Briefly, 1 ml of beads were added and slowly stirred over night at 4° C. Beads were washed 3× with 10 ml PBS in a gravity flow column. Protein was eluted with 5 ml of 0.1 M Glycine, pH3.0 and immediately neutralized by adding 1 ml Tris/HCL pH8.0. Elution fractions were dialyzed against 21 of PBS at 4° C. for a total of 3 times. Then, size exclusion chromatography (AKTA Pure 25, GE Healthcare Life Science) using a HiLoad 26/600 Superdex 200 pg column (GE Healthcare Life Science) at a flowrate of 1 ml/min (PBS buffer) was performed to isolate monomeric antibodies. Antibody preparations were analyzed by SDS-PAGE using standard procedures. Gels were stained with Coomassie blue. Protein concentration of purified proteins was analyzed by the BCA assay (Pierce) according to the manufacturer's conditions.

Flow cytometry: 0.5×10⁶ cells were used for individual staining reactions. Cells are washed once in 1 ml of PBA (PBS, 1% BSA, 0.05% NaN₃). The cell pellet is resuspended with 50 μl of purified recombinant protein at a concentration of 50 μg/ml diluted in PBA. Cells are incubated on ice for 30 min. Cells are washed two times in 1 ml of PBA. Then the cell pellet is resuspended in 25 μl of a 1:20 dilution of anti-human-IgGFITC (Jackson Immuno Research, cat. no.: 109-096-098) and incubated for 30 min on ice in the dark. Then cells are washed twice in 1 ml PBA. Cells are finally resuspended in 500 μl PBA and immediately analyzed on a Navios flow cytometer (Beckman Coulter).

### EXAMPLES

### Example 1: Cloning of Bispecific Antibodies (FIG. 5)

The bispecific CD277 antibodies are made according to the following procedure: Expression vectors for the production of IgG-scFv molecules were designed by standard procedures Kellner, C S. et al.; Methods Mol Biol, 2018. 1827: p. 381-397). pSEC-Tag2-Hygro-C was used as the backbone for the generation of mammalian expression vectors. IgG-scFv antibody derivatives were designed in a modified format based on the prototype IgG-scFv format originally described by Coloma, M. J. et al.; Nat Biotechnol, 1997. 15(2): p. 159-63.

Light chain: Light chain design was realized as described by Kellner, C S. et al.; supra. For the light chain expression cassette, a secretion leader sequence (Li; Haryadi, R S. et al; PLoS One, 2015. 10(2): p. e0116878) was added to the 5′-end of the VL-region. A human C-kappa region was fused at the 3′-end to form a full kappa light chain coding sequence. A minimal Kozak sequence was added upstream of the start codon to allow optimal initiation of translation. NheI and PmeI restriction sites were introduced at the 5′- and 3′-end, respectively. Cloning in the vector backbone was performed according to standard procedures.

Heavy chain derivative: The heavy chain derivative is coding for a IgG1 heavy chain carrying L234A and L235A amino acid exchanges in the lower hinge region to prevent Fc receptor interaction (Lund, J G. et al.; J Immunol, 1991. 147(8): p. 2657-62). A heavy chain secretion leader was added to the 5′-end of the VH region (H7; Haryadi, R S. et al; PLoS One, 2015. 10(2): p. e0116878). A minimal Kozak sequence was added upstream of the start codon allowing optimal initiation of translation. The stop codon of the IgG heavy chain was removed and a sequence coding for a 15 amino acid flexible linker (G₄S)₃ (SEQ ID NO:97) was introduced. The last two codons of the flexible linker (GS) at the DNA level harbors a BamHI restriction site followed by a PmeI restriction site. The respective scFv fragments were designed in the VL-(G₄S)₄-VH format as BamHI-PmeI cloning cassettes. Cloning of the final expression constructs was performed according to standard procedures.

In the heavy chain derivative additional restrictions sites not affecting amino acid composition were introduced to allow a modular design and exchange of specific parts of the molecule:
NheI-PpuMI: exchange of the VH-region.
PpuMI-BsrGI: Exchange of silencing mutations in the CH2 domain.
BsrGI-BamHI: exchange of linker sequences.
BamHI-PmeI: exchange of scFv fragments.

TABLE 2a
Bispecific CD277 antibodies, comprising as first
binding part the parent anti-CD277 antibody

| | | | | | | SEQ | SEQ |
|---|---|---|---|---|---|---|---|
| | | SEQ | SEQ | SEQ | SEQ | ID | ID |
| | | ID | ID | ID | ID | IgG | IgG |
| Label | Molecule | VL2 | VH2 | VL1 | VH1 | LC | HC |
| EvB#3 | CD277- | 18 | 22 | 5 | 1 | 93 | 92 |
| | STEAP1 | | | | | | |
| EvB#4 | CD277- | 26 | 30 | 5 | 1 | 93 | 102 |
| | DLL3 | | | | | | |
| EvB#5 | CD277- | 10 | 14 | 5 | 1 | 93 | 9 |
| | FOLR1 | | | | | | |
| EvB#6 | CD277- | 34 | 38 | 5 | 1 | 93 | 103 |
| | CLDN18.2 | | | | | | |

TABLE 2b
Bispecific CD277 antibodies, comprising
as first binding part antibody 47

| | SEQ ID | SEQ ID | SEQ ID | SEQ ID | SEQ ID |
|---|---|---|---|---|---|
| Molecule | VL2 | VH2 | VL1 | VH1 | IgG LC |
| CD277- | 18 | 22 | 5 | 42 | 93 |
| STEAP1 | | | | | |
| CD277- | 26 | 30 | 5 | 42 | 93 |
| DLL3 | | | | | |
| CD277- | 10 | 14 | 5 | 42 | 93 |
| FOLR1 | | | | | |
| CD277- | 34 | 38 | 5 | 42 | 93 |
| CLDN18.2 | | | | | |

TABLE 2c
Bispecific CD277 antibodies, comprising
as first binding part antibody 52

| | SEQ ID | SEQ ID | SEQ ID | SEQ ID | SEQ ID |
|---|---|---|---|---|---|
| Molecule | VL2 | VH2 | VL1 | VH1 | IgG LC |
| CD277- | 18 | 22 | 65 | 42 | 93 |
| STEAP1 | | | | | |
| CD277- | 26 | 30 | 65 | 42 | 93 |
| DLL3 | | | | | |
| CD277- | 10 | 14 | 65 | 42 | 93 |
| FOLR1 | | | | | |
| CD277- | 34 | 38 | 65 | 42 | 93 |
| CLDN18.2 | | | | | |

Antibody 47:VL parent, VH CDR2 N185S, K190N, all other VH/VL CDRs same as parent
Antibody 52:VL CDR1 L31V, VH CDR2 N185S, K190N, all other VH/VL CDRs same as parent

### Example 2: Generation of Humanized Bispecific Antibodies

Affinity maturation of antibodies is a stepwise process during an immune response. By accumulating additional mutations in the CDR regions compared to germline and undergoing a strict selection process, antibody affinity may increase in a stepwise fashion as described by Rajewsky and co-workers in 1988 (Allen, D., et al.; EMBO J, 1988. 7(7): p. 1995-2001, Kocks, C. and K. Rajewsky, Proc Natl Acad Sci USA, 1988. 85(21): p. 8206-10. Therefore, by a stepwise restoration of germline configuration within the light and heavy chain variable regions, humanized antibodies of the invention can be generated.

### Method of Humanization

The humanization of murine monoclonal antibody was performed using standard CDR-grafting technology. The principle of this method is to reshape a human antibody containing only the complementarity determining regions (CDRs) from the murine monoclonal antibody with the aim of reducing immunogenicity when used as a therapeutic in humans. Humanization by CDR-grafting requires that the antigen-binding residues from the murine antibody be retained in the humanized antibody; thus, the identification of these residues obviously plays an important role in the protocol. To guide the humanization process and help in the decision to conserve parental murine residues or substitute them with their human germline counterparts, the 4F9L X-ray structure of murine monoclonal antibody scFv with its antigen BTN3A1 was used.

The CDR-grafting protocol used is a modernized version of the approach pioneered by Greg Winter and colleagues at the Medical Research Council, Cambridge, UK. The definition of the CDRs is based on the Kabat nomenclature. The selection of human framework acceptor regions into which murine monoclonal antibody murine CDR regions are grafted was accomplished by searching the IMGT murine and human V gene databases using IgBLAST, developed at NCBI to facilitate analysis of immunoglobulin V region sequences (http://www.ncbi.nlm.nih.gov/igblast/), with murine monoclonal antibody murine variable region sequences as input. The applied strategy is to use the human germline sequences that are natural human sequences not containing the idiosyncratic somatic mutations found in individual human antibody sequences.

Light chain backmutation: The variable region of the light chain from the first binding part of the parental antibody was compared to the germline repertoire of the mouse (IMGT database) and the germline line genes demonstrating closest homology were identified. Thereby, IGKV15-103*01 and IGKJ2*01 [F] genes were identified and aligned to the parental VL region. Six amino acid residues were identified to be different from germline. By illustrating the identified residues in the crystal structure of the parental antibody, amino acid residues being surface exposed and may therefore directly contribute to antigen interaction were identified. Individual amino acids or clusters of amino acids were converted to germline configuration and used for the generation of expression constructs.

Heavy chain backmutation: A similar strategy was applied to identify potential amino acid positions in the heavy chain variable region. IGHV1S81*02 [F]v-gene was identified as closest match. No D and J segment was identified, since the CDR3 region seemed to be highly mutated making identification of corresponding gene segments difficult. Similar to the strategy applied to reset the VL-region to germline, mutations in the CDR1 and CDR2 region of the heavy chain were reverted to germline configuration in a stepwise fashion (single mutations or clusters). To identify residues in the CDR3 a different strategy was applied since no homologous germline gene segments could be identified. Here, surface exposed residues in the CDR3 region were identified by analysis of the co-crystal structure of 20.1 and BNT3A residues (Payne, K K et al.; Science, 2020. 369(6506): p. 942-949). Three of these residues have been described as potential contact residues (Payne, K K et al.; supra) and were therefore converted to alanine. Alanine exchange was chosen because alanine scanning has been described to identify residues critical in epitope binding by disrupting antibody/antigen interactions (Parhami-Seren, B M. et al; J Immunol, 2001. 167(9): p. 5129-35). Individual amino acids or clusters of amino acids were converted to germline configuration and used for the generation of expression constructs.

TABLE 3
First binding part of the bispecific antibody according to the invention

| | | CDRL1, | | | CDRH1, | |
|---|---|---|---|---|---|---|
| | VL | 2, 3 | | VH | 2, 3 | |
| | SEQ ID | SEQ ID | VL | SEQ ID | SEQ ID | VH |
| Molecule | NO | NO | Description | NO | NO | Description |
| EvB# | 5 | 6, 7, 8 | Parent | 1 | 2, 3, 4 | Parent |
| 1 | | | (reference) | | | reference |
| EvB# | 5 | 6, 7, 8 | Parent | 54 | 55, 56, | cluster |
| 21 | | | | | 57 | 1 |
| EvB# | 5 | 6, 7, 8 | Parent | 58 | 59, 60, | cluster |
| 22 | | | | | 61 | 2 |
| EvB# | 5 | 6, 7, 8 | Parent | 42 | 43, 44, | N185S- |
| 23 | | | | | 45 | K190N |
| (EvB# | | | | | | |
| 47) | | | | | | |
| EvB# | 5 | 6, 7, 8 | Parent | 46 | 47, 48, | R162S- |
| 24 | | | | | 49 | Y164W |
| EvB# | 5 | 6, 7, 8 | Parent | 62 | 63, 64, | VH- |
| 43 | | | | | 65 | 46_1A |
| EvB# | 5 | 6, 7, 8 | Parent | 66 | 67, 68, | VH- |
| 44 | | | | | 69 | 46_1B |
| EvB# | 5 | 6, 7, 8 | Parent | 70 | 71, 72, | VH- |
| 45 | | | | | 73 | 46_1C |
| EvB# | 78 | 79, 80, | L31V- | 1 | 2, 3, 4 | Parent |
| 26 | | 81 | H92Q- | | | |
| | | | Y96L | | | |
| EvB# | 86 | 87, 88, | L31V | 1 | 2, 3.4 | Parent |
| 28 | | 89 | | | | |
| EvB# | 86 | 87, 88, | L31V | 42 | 43, 44, | N185S- |
| 29 | | 89 | | | 45 | K190N |
| (EvB# | | | | | | |
| 52) | | | | | | |
| EvB# | 86 | 87, 88, | L31V | 46 | 47, 48, | R162S- |
| 30 | | 89 | | | 49 | Y164W |
| EvB# | 82 | 83, 84, | H92Q- | 42 | 43, 44, | N185S- |
| 31 | | 85 | Y96L | | 45 | K190N |
| EvB# | 82 | 83, 84, | H92Q- | 1 | 2, 3. 4 | Parent |
| 27 | | 85 | Y96L | | | |
| EvB# | 82 | 83, 84, | H92Q- | 46 | 47, 48, | R162S- |
| 32 | | 85 | Y96L | | 49 | Y164W |
| EvB# | 78 | 79, 80, | L31V- | 42 | 43, 44, | N185S- |
| 33 | | 81 | H92Q- | | 45 | K190N |
| | | | Y96L | | | |
| EvB# | 78 | 79, 80, | L31V- | 46 | 47, 48, | R162S- |
| 34 | | 81 | H92Q- | | 49 | Y164W |
| | | | Y96L | | | |
| EvB# | 78 | 79, 80, | L31V- | 54 | 55, 56, | cluster |
| 35 | | 81 | H92Q- | | 57 | 1 |
| | | | Y96L | | | |
| EvB# | 78 | 79, 80, | L31V- | 58 | 59, 60, | cluster |
| 36 | | 81 | H92Q- | | 61 | 2 |
| | | | Y96L | | | |
| EvB# | 78 | 79, 80, | L31V- | 50 | 51, 52, | cluster |
| 37 | | 81 | H92Q- | | 53 | 1-2 |
| | | | Y96L | | | |
| EvB# | 74 | 75, 76, | L31V- | 42 | 43, 44, | N185S- |
| 38 | | 77 | R39K- | | 45 | K190N |
| | | | R50K- | | | |
| | | | A68G- | | | |
| | | | H92Q- | | | |
| | | | Y96L | | | |
| EvB# | 74 | 75, 76, | L31V- | 1 | 2, 3, 4 | Parent |
| 25 | | 77 | R39K- | | | |
| | | | R50K- | | | |
| | | | A68G- | | | |
| | | | H92Q- | | | |
| | | | Y96L | | | |
| EvB# | 74 | 75, 76, | L31V- | 46 | 47, 48, | R162S- |
| 39 | | 77 | R39K- | | 49 | Y164W |
| | | | R50K- | | | |
| | | | A68G- | | | |
| | | | H92Q- | | | |
| | | | Y96L | | | |
| EvB# | 74 | 75, 76, | L31V- | 54 | 55, 56, | cluster |
| 40 | | 77 | R39K- | | 57 | 1 |
| | | | R50K- | | | |
| | | | A68G- | | | |
| | | | H92Q- | | | |
| | | | Y96L | | | |
| EvB# | 74 | 75, 76, | L31V- | 58 | 59, 60, | cluster |
| 41 | | 77 | R39K- | | 61 | 2 |
| | | | R50K- | | | |
| | | | A68G- | | | |
| | | | H92Q- | | | |
| | | | Y96L | | | |
| EvB# | 74 | 75, 76, | L31V- | 50 | 51, 52, | cluster |
| 42 | | 77 | R39K- | | 53 | 1-2 |
| | | | R50K- | | | |
| | | | A68G- | | | |
| | | | H92Q- | | | |
| | | | Y96L | | | |
| EvB# | 62 | 63, 64, 65 | VH- | 116 | 117, 118, | VL- |
| 101 | | | 46_1A | | 119 | 12_1A |
| EvB# | 62 | 63, 64, 65 | VH- | 120 | 121, 122, | VL- |
| 102 | | | 46_1A | | 123 | 12_1B |
| EvB# | 62 | 63, 64, 65 | VH- | 124 | 125, 126, | VL- |
| 103 | | | 46_1A | | 127 | 12_1C |
| EvB# | 62 | 63, 64, 65 | VH- | 128 | 129, 130, | VL- |
| 104 | | | 46_1A | | 131 | 11_1A |
| EvB# | 62 | 63, 64, 65 | VH- | 132 | 133, 134, | VL- |
| 105 | | | 46_1A | | 135 | 11_1B |
| EvB# | 62 | 63, 64, 65 | VH- | 136 | 137, 138, | VL- |
| 106 | | | 46_1A | | 139 | 11_1C |
| EvB# | 66 | 67, 68, 69 | VH- | 116 | 117, 118, | VL- |
| 107 | | | 46_1B | | 119 | 12_1A |
| EvB# | 66 | 67, 68, 69 | VH- | 120 | 121, 122, | VL- |
| 108 | | | 46_1B | | 123 | 12_1B |
| EvB# | 66 | 67, 68, 69 | VH- | 124 | 125, 126, | VL- |
| 109 | | | 46_1B | | 127 | 12_1C |
| EvB# | 66 | 67, 68, 69 | VH- | 128 | 129, 130, | VL- |
| 110 | | | 46_1B | | 131 | 11_1A |
| EvB# | 66 | 67, 68, 69 | VH- | 132 | 133, 134, | VL- |
| 111 | | | 46_1B | | 135 | 11_1B |
| EvB# | 66 | 67, 68, 69 | VH- | 136 | 137, 138, | VL- |
| 112 | | | 46_1B | | 139 | 11_1C |
| EvB# | 70 | 71, 72, 73 | VH- | 116 | 117, 118, | VL- |
| 113 | | | 34_1A | | 119 | 12_1A |
| EvB# | 70 | 71, 72, 73 | VH- | 120 | 121, 122, | VL- |
| 114 | | | 34_1A | | 123 | 1_1B |
| EvB# | 70 | 71, 72, 73 | VH- | 124 | 125, 126, | VL- |
| 115 | | | 34_1A | | 127 | 12_1C |
| EvB# | 70 | 71, 72, 73 | VH- | 128 | 129, 130, | VL- |
| 116 | | | 34_1A | | 131 | 11_1A |
| EvB# | 70 | 71, 72, 73 | VH- | 132 | 133, 134, | VL- |
| 117 | | | 34_1A | | 135 | 11_1B |
| EvB# | 70 | 71, 72, 73 | VH- | 136 | 137, 138, | VL- |
| 118 | | | 34_1A | | 139 | 11_1C |
| EvB# | 104 | 105, 106, | VH- | 116 | 117, 118, | VL- |
| 119 | | 107 | 34_1B | | 119 | 12_1A |
| EvB# | 104 | 105, 106, | VH- | 120 | 121, 122, | VL- |
| 120 | | 107 | 34_1B | | 123 | 12_1B |
| EvB# | 104 | 105, 106, | VH- | 124 | 125, 126, | VL- |
| 121 | | 107 | 34_1B | | 127 | 12_1C |
| EvB# | 104 | 105, 106, | VH- | 128 | 129, 130, | VL- |
| 122 | | 107 | 34_1B | | 131 | 11_1A |
| EvB# | 104 | 105, 106, | VH- | 132 | 133, 134, | VL- |
| 123 | | 107 | 34_1B | | 135 | 11_1B |
| EvB# | 104 | 105, 106, | VH- | 136 | 137, 138, | VL- |
| 124 | | 107 | 34_1B | | 139 | 11_1C |
| EvB# | 108 | 109, 110, | VH- | 116 | 117, 118, | VL- |
| 125 | | 111 | 34_1C | | 119 | 12_1A |
| EvB# | 108 | 109, 110, | VH- | 120 | 121, 122, | VL- |
| 126 | | 111 | 34_1C | | 123 | 12_1B |
| EvB# | 108 | 109, 110, | VH- | 124 | 125, 126, | VL- |
| 127 | | 111 | 34_1C | | 127 | 12_1C |
| EvB# | 108 | 109, 110, | VH- | 128 | 129, 130, | VL- |
| 128 | | 111 | 34_1C | | 131 | 11_1A |
| EvB# | 108 | 109, 110, | VH- | 132 | 133, 134, | VL- |
| 129 | | 111 | 34_1C | | 135 | 11_1B |
| EvB# | 108 | 109, 110, | VH- | 136 | 137, 138, | VL- |
| 130 | | 111 | 34_1C | | 139 | 11_1C |
| EvB# | 112 | 113, 114, | VH- | 116 | 117, 118, | VL- |
| 131 | | 115 | 34_1D | | 119 | 12_1A |
| EvB# | 112 | 113, 114, | VH- | 120 | 121, 122, | VL- |
| 132 | | 115 | 34_1D | | 123 | 12_1B |
| EvB# | 112 | 113, 114, | VH- | 124 | 125, 126, | VL- |
| 133 | | 115 | 34_1D | | 127 | 12_1C |
| EvB# | 112 | 113, 114, | VH- | 128 | 129, 130, | VL- |
| 134 | | 115 | 34_1D | | 131 | 11_1A |
| EvB# | 112 | 113, 114, | VH- | 132 | 133, 134, | VL- |
| 135 | | 115 | 34_1D | | 135 | 11_1B |
| EvB# | 112 | 113, 114, | VH- | 136 | 137, 138, | VL- |
| 136 | | 115 | 34_1D | | 139 | 11_1C |

Cluster 1:: R162S, Y164W, L165M, Y166H
Cluster 2:: N185S, G188R, K190N, F191Y
Cluster 1-2:: R162S, Y164W, L165M, Y166H, N185S, G188R, K190N, F191Y

A heavy and light variable chain set of a bispecific antibody according to the invention is defined as the two chains of one line of the table. “R162S” means that amino acid R at position 162 is replaced by amino acid S. N185S means that asparagine on position 185 is replaced by serine. N185S and K190N are in bold and underlined in SEQ ID NO:44. L31V is in bold and underlined in SEQ ID NO:75 Respective meaning for all other similar terms. Counting of other amino acids in the variable chains can start using N185 and L31.

### Example 3: Generation and Characterization of Activated Vγ9Vδ2 T Cell Lines

To generate expanded γδ T cell lines, 10⁶ cells/mL from leukocyte concentrates (LRS) from healthy adult blood donors were cultured in 6-well plates in complete medium with 50 IU/mL rIL-2 (Novartis, Basel, Switzerland) and stimulated with 2.5 μM aminobisphosphonate (n-BP) zoledronic acid (Novartis), which induces a selective outgrowth of Vγ9V62-expressing γδ T cells. Since resting, initially stimulated γδ T cells produced only very low amounts of IL-2, 50 IU/mL (15 μg/mL) rIL2 was added every other day (Oberg et al., Cancer Res. 2014). After two weeks, a selective expansion of γδ T cells expressing a Vδ2 chain with a purity>94% was observed. The Vδ2 T cell activation was indicated by a slightly enhanced CD25 expression (Pechhold et al. J Immunol Baltim Md 1950 152, 4984-92 (1994)) and a strong up-regulation of activation marker CD69. Additionally, the increased Vδ2 T cell population revealed a central memory- (CM, CD27+CD45RA−) or effector memory (EM, CD27− CD45RA−) phenotype demonstrating the activation of these expanded γδ T cells.

### Example 4: Selection of Tumor Cell Lines

To test the diverse antibody constructs, a panel of different tumor cell lines which express the respective tumor-antigen were selected based on published information on antigen expression levels or on FACS analysis. Briefly, for the surface staining, 3 to 5×10⁵ cells were washed twice with washing buffer (PBS containing 1% BSA, 0.100 NaN₃). Thereafter, cells were stained with fluorochrome-conjugated or unconjugated antibodies or isotype controls for 25 minutes following the procedures outlined by the manufacturer, washed twice and resuspended in 1% PFA (paraformaldehyde) in PBS buffer or stained with a second step antibody. After incubation with a second step antibody cells were washed twice and resuspended in 1% PFA buffer. All samples were analyzed on a LSR-Fortessa flow cytometer (BD Biosciences) using Diva9 and FlowJo software. Result of the literature and FACS analysis are summarized in table 4.

TABLE 4
Expression of tumor-antigens on tumor cells

| | | Expression of | | | | |
|---|---|---|---|---|---|---|
| Tumor cells | | CD277 | FOLR1 | DLL3 | STEAP1 | CLDN18.2 |
| OVCAR- | literature | Yes (1) | Yes | | | |
| 3 | | | (2, 3) | | | |
| Ovarian | FACS | weak | positive | | | |
| Ca | | | | | | |
| NCI- | literature | | | | | |
| H1693 | | | | | | |
| NSCLC | FACS | positive | | | | |
| UM-UC- | literature | Yes (4) | | | Yes (5) | |
| 3 | | | | | | |
| Bladder | FACS | positive | | weak | weak | |
| Ca | | | | | | |
| PA-TU- | literature | | | | | Yes (6) |
| 8988s | | | | | | |
| Pancreas | FACS | positive | | weak | | weak |
| Ca | | | | | | |
| NCI- | literature | | | Yes | | |
| H510 | | | | (7) | | |
| SCLC | FACS | | | weak | | |
| BxPC3 | literature | Yes (8) | | | | Yes (6) |
| PDAC | FACS | positive | | | | |

* = not disclosed
Literature: 1. Payne, K. K. et al. Science 369, 942-949 (2020);
2. Ebel, W. et al. Cancer Immun 7, 6 (2007);
3. Shivange, G. et al. Cancer Cell 34, 331-345.e11 (2018);
4. Shimizu, T., et al., Oncoimmunology 7, e1424671 (2018),
5. Gomes, I. M., et al., Mol Cancer Res 10, 573-587 (2012),
6. Türeci, Özlem, et al., Oncoimmunology 8, e1523096 (2018),
7. Hipp, S. et al. Clin Cancer Res Official J Am Assoc Cancer Res 26, 5258-5268 (2020), Benyamine, A. et al. Oncoimmunology 7, 00-00 (2017).

### Example 5: Generation of Knock Out Tumor Cell Lines

Generation of tumor-antigen KO cells by RNP transfection: Guide RNA (gRNA) was prepared by combining a respective crRNA and tracrRNA at equimolar concentrations (100 μM), annealing at 95° C. for 5 min and renaturation at room temperature. The RNPs were subsequently prepared by combining the gRNAs and recombinant S.p. Cas9 protein in PBS and incubating at room temperature for 15 min. RNPs were electroporated into parental cells using the SF Cell Line 4D-Nucleofector X Kit S (Lonza; #V4XC-2032) and a 4D-Nucleofector X unit (Lonza) following the manufacturer's instructions and program FE-132. Monoclonal cells were subsequently generated by FACS sorting (BD Aria), expanded, and validated by flow cytometry stainings and amplicon sequencing (NGS).

Generation of tumor-antigen KO cells by lentivirus transfection: Parental cells were first transduced with a Cas9-p2A-Blasticidin-lentivirus and selected with Blasticidin to achieve a stable expression of Cas9. A respective guide RNA was then cloned into the CROP-seq-Guide-Puro plasmid (Addgene #86708) and a lentivirus was produced. Blasticidin-selected Cas-9 expressing cells were subsequently transduced with said lentivirus, cells were selected with Puromycin, expanded, and validated by flow cytometry stainings and amplicon sequencing (NGS). As a control, the same transfection protocol was applied using non-targeted guide RNA (“sgNT”) generating the respective sgNT cell line. A suitable guide RNA sequence for the generation of FOLR1 KO cells is SEQ ID NO:96.

### Example 6: Cell Lysis Assay

The cytotoxicity against tumor cell lines such as OVCAR-3 (ovarian cancer), NCI-H1693 (NSCLC), or UM-UC-3 (bladder cancer) was determined by a Real-Time Cell Analyzer (RTCA, X-Celligence, ACEA Biosciences, San Diego, CA, USA) in triplicates as described elsewhere (Oberg et al., 2014 and 2020). Briefly, 7.5-10×10³ adherent tumor cells/well in complete medium RPMI 1640 (supplemented with 25 mM HEPES, 2 mM L-glutamine, 100 μg/mL streptomycin, 100 U/mL penicillin and 10% fetal bovine serum) were added to 96-well micro-E-plate to monitor the impedance of the tumor cells via electronic sensors every five minutes for up to ˜24-40 h. The measured impedance of the tumor cells is expressed as an arbitrary unit called cell index (“CI”), which reflects changes in cellular parameters such as morphological changes (e.g. adherence, spreading), cell proliferation and cell lysis. Since the initial adherence of tumor cells in different wells can differ slightly, the CI can be normalized to 1 after tumor cancer cells having reached their linear growth phase. When linear growth rate was reached after ˜24-40 h, activated Vγ9Vδ2 T lymphocytes at an E/T ratio of 5:1 were added as well as medium containing 12.5 IU/mL rIL-2 and the various antibody constructs at the indicated concentrations or various controls (“start of experiment”, t=0 h). For controls, tumor cells were treated in several wells with a final concentration of 1% Triton X-100 as a positive control for complete lysis, and in several other wells with activated Vγ9Vδ2 T lymphocytes (same conditions as above) as a control for background lysis. The lysis of adherent tumor cells was monitored by measuring the normalized CI for at least 3 minutes at different timepoints.

By using the RTCA software (ACEA Biosciences Inc.), the raw data files were exported to Microsoft Excel or Graph Pad Prism for further evaluation. The mean CI of Triton-X-100 samples and of Vγ9Vδ2 T lymphocytes without antibody additions were calculated at the indicated time points after start of experiment and defined as complete lysis (“Triton X 100”) and background lysis (“Medium Ctrl”), respectively. The tumor cell lysis induced by antibody constructs was calculated for each sample at the same time points (“tx”) as tumor cell:

$$lysis ( tx ) = ( CI ( tx ) - Medium Ctrl ( tx ) / ( Triton X 100 - Medium Ctrl ( tx ) ) * 100$$

Curve fitting was performed by using the sigmoidal dose-response function with Graphpad Prism 9 providing the best-fit value for maximum tumor cell lysis(tx) achieved with Reference antibody (Top value). % tumor cell lysis relative to maximum tumor cell lysis (“Top”) achieved by the Reference antibody was calculated by the following formula: % tumor cell lysis(tx)=tumor cell lysis(tx)/Top*100
Oberg, H. H.; et al.; Front. Immunol. 2014, 5, 643 and Oberg, H. H.; et al.; Methods Enzymol. 2020, 631, 429-441. Results are shown in FIGS. 4, 6 and 8.

### Example 7: SPR Assay

SPR assay was performed according to the state of the art. Results for the reference antibody are shown in tables 5. In short, recombinant CD277 was immobilized to the surface of a Biacore CM5 optical sensor chip by covalent EDC/NHS coupling following the Biacore amine coupling kit protocol. Antibody samples were applied as analytes in serial dilution allowing standardized comparison of all antibodies binding to the identical target molecule surface. Kinetic analysis data are based on 1:1 Langmuir curve fitting model and mean Langmuir on-rates, off-rates and KD values: see table 5.

TABLE 5

| VL modi- | VH modi- | | | |
|---|---|---|---|---|
| fication | fication | ka | kd | KD (nM) |
| none | none | 3.69 ± 0.54E+4 | 1.04 ± 0.20E−4 | 2.80 ± 0.31 |
| none | N53S, K58N | 1.58 ± 0.22E+4 | 4.09 ± 0.48E−4 | 26.30 ± 4.72 |
| L31V | N53S, K58N | 2.79 ± 0.13E+4 | 3.85 ± 0.23E−4 | 13.90 ± 1.33 |

E+4 means 10⁴,
E−4 means 10⁻⁴

### Example 8: Degranulation and Cell Death Assay

Principle: Cytotoxic T cells such as γδ T cells store cytotoxic mediators such as granzymes, perforins and granulysin in secretory lysosomes. Lysosome-associated membrane glycoproteins (LAMP) such as LAMP-1 (CD107a) and LAMP-2 (CD107b) are embedded in the lipid bilayer membrane of secretory lysosomes. After activation of T cells, secretory lysosomes can move towards the cell membrane and fuse with it. After fusion, LAMPs are transiently expressed on the cell surface of T cells, and secretory lysosomes degranulate their granule content.

Method: Short-term activated γδ T cells were cultured in RPMI 1640 medium supplemented with 2 mM L-glutamine, 25 mM Hepes, 100 U/mL penicillin, 100 μg/mL streptomycin, 10% fetal calf serum under regular conditions (5% CO₂, humidified, 37° C.). γδ T cells supplemented with 12.5 U/mL IL-2 were cultured together with medium, 300 nM bromohydrin pyrophosphate, different concentrations of constructs or with control construct AV #75 in 96-well microtiter plates (Nunc, Wiesbaden) for six hours. For CD107-assay, 0.5 μg/mL PE-labeled anti-CD107a mAb clone H4A3 (Biolegend) and 0.5 μg/mL PE-labeled anti-CD107b mAb clone H4B4 (Biolegend) or appropriate isotype controls were added directly to the 96-well microtiter plates, whereas 3 μM secretion inhibitor monensin was added three hours after culturing the cells. After additional three hours, γδ T cells were washed and stained with PerCP-labeled anti-CD45 mAb (clone 2D1, BD Biosciences), AlexaF700-labeled anti-CD3 mAb (clone SK7, Biolegend), BV510-labeled anti-CD8 mAb (clone SK1, BD Biosciences), PE-Cy7-labeled anti-TCR γδ mAb (clone 11F2, BD Biosciences) and APC-Vio770-labeled anti-Vδ2 (clone REA 771, Miltenyi), washed, and taken up in PBS with SYTOX™ Green Dead Cell Stain (1:4000, Thermo Scientific, #S34860) 20 min. before analyzing cells by flow cytometry (LSR Fortessa, BD Biosciences). Results for γδ T cells of 4 different donors are shown in FIG. 9.

TABLE 6
CDR Sequences of humanized EvB#47 (CDRH/CDRL sets)

| No. | CDRH1 | SEQ | CDRH2 | SEQ | CDRH3 | SEQ | CDRL1 | SEQ | CDRL2 | SEQ | CDRL3 | SEQ |
|---|---|---|---|---|---|---|---|---|---|---|---|---|
| EvB47 | RYYLY | 2, 43 | EINPSNGGTNFNEKFKS | 44 | EDDYDGTPDAMDY | 4, 45 | HASQNINLWLS | 6 | RASNLHT | 7 | QGHSYPYT | 8 |
| murine | | | | | | | | | | | | |
| 2 | RYYMY | 67 | EINPSNGGTNFNEKFKS | 44 | EDDYDGTPDAMDY | 4 | HASQNINLWLS | 6 | RASNLHT | 7 | QGHSYPYT | 8 |
| 3 | RYYMY | 67 | EINPSNGGTNFAQKFQG | 68 | EDDYDGTPDAMDY | 4 | HASQNINLWLS | 6 | RASNLHT | 7 | QGHSYPYT | 8 |
| 4 | RYYLY | 59 | EINPSNGGTNFNEKLKS | 72 | EDDYDGTPDAMDY | 4 | HASQNINLWLS | 6 | RASNLHT | 7 | QGHSYPYT | 8 |
| 5 | RYYWY | 105 | EINPSNGGTNFNEKLKS | 72 | EDDYDGTPDAMDY | 4 | HASQNINLWLS | 6 | RASNLHT | 7 | QGHSYPYT | 8 |
| 6 | RYYLY | 59 | EINPSNGGTNFNESLKS | 110 | EDDYDGTPDAMDY | 4 | HASQNINLWLS | 6 | RASNLHT | 7 | QGHSYPYT | 8 |
| 7 | RYYWY | 105 | EINPSNGGTNFNESLKS | 110 | EDDYDGTPDAMDY | 4 | HASQNINLWLS | 6 | RASNLHT | 7 | QGHSYPYT | 8 |
| 8 | RYYLY | 2 | EINPSNGGTNFNEKFKS | 44 | EDDYDGTPDAMDY | 4 | RASQGISSWLS | 121 | RASNLHT | 7 | QGHSYPYT | 8 |
| 9 | RYYMY | 67 | EINPSNGGTNFNEKFKS | 44 | EDDYDGTPDAMDY | 4 | RASQGISSWLS | 121 | RASNLHT | 7 | QGHSYPYT | 8 |
| 10 | RYYMY | 67 | EINPSNGGTNFAQKFQG | 68 | EDDYDGTPDAMDY | 4 | RASQGISSWLS | 121 | RASNLHT | 7 | QGHSYPYT | 8 |
| 11 | RYYLY | 2 | EINPSNGGTNFNEKLKS | 72 | EDDYDGTPDAMDY | 4 | RASQGISSWLS | 121 | RASNLHT | 7 | QGHSYPYT | 8 |
| 12 | RYYWY | 105 | EINPSNGGTNFNEKLKS | 72 | EDDYDGTPDAMDY | 4 | RASQGISSWLS | 121 | RASNLHT | 7 | QGHSYPYT | 8 |
| 13 | RYYLY | 2 | EINPSNGGTNFNESLKS | 110 | EDDYDGTPDAMDY | 4 | RASQGISSWLS | 121 | RASNLHT | 7 | QGHSYPYT | 8 |
| 14 | RYYWY | 105 | EINPSNGGTNFNESLKS | 110 | EDDYDGTPDAMDY | 4 | RASQGISSWLS | 121 | RASNLHT | 7 | QGHSYPYT | 8 |
| 15 | RYYLY | 2 | EINPSNGGTNFNEKFKS | 44 | EDDYDGTPDAMDY | 4 | RASQSVSSWLS | 133 | RASNLHT | 7 | QGHSYPYT | 8 |
| 16 | RYYMY | 67 | EINPSNGGTNFNEKFKS | 44 | EDDYDGTPDAMDY | 4 | RASQSVSSWLS | 133 | RASNLHT | 7 | QGHSYPYT | 8 |
| 17 | RYYMY | 67 | EINPSNGGTNFAQKFQG | 68 | EDDYDGTPDAMDY | 4 | RASQSVSSWLS | 133 | RASNLHT | 7 | QGHSYPYT | 8 |
| 18 | RYYLY | 2 | EINPSNGGTNFNEKLKS | 72 | EDDYDGTPDAMDY | 4 | RASQSVSSWLS | 133 | RASNLHT | 7 | QGHSYPYT | 8 |
| 19 | RYYWY | 105 | EINPSNGGTNFNEKLKS | 72 | EDDYDGTPDAMDY | 4 | RASQSVSSWLS | 133 | RASNLHT | 7 | QGHSYPYT | 8 |
| 20 | RYYLY | 2 | EINPSNGGTNFNESLKS | 110 | EDDYDGTPDAMDY | 4 | RASQSVSSWLS | 133 | RASNLHT | 7 | QGHSYPYT | 8 |
| 21 | RYYWY | 105 | EINPSNGGTNFNESLKS | 110 | EDDYDGTPDAMDY | 4 | RASQSVSSWLS | 133 | RASNLHT | 7 | QGHSYPYT | 8 |
| 22 | RYYLY | 2 | EINPSNGGTNFNEKFKS | 44 | EDDYDGTPDAMDY | 4 | RASQSVSSWLS | 133 | RASNRHT | 138 | QGHSYPYT | 8 |
| 23 | RYYMY | 67 | EINPSNGGTNFNEKFKS | 44 | EDDYDGTPDAMDY | 4 | RASQSVSSWLS | 133 | RASNRHT | 138 | QGHSYPYT | 8 |
| 24 | RYYMY | 67 | EINPSNGGTNFAQKFQG | 68 | EDDYDGTPDAMDY | 4 | RASQSVSSWLS | 133 | RASNRHT | 138 | QGHSYPYT | 8 |
| 25 | RYYLY | 2 | EINPSNGGTNFNEKLKS | 72 | EDDYDGTPDAMDY | 4 | RASQSVSSWLS | 133 | RASNRHT | 138 | QGHSYPYT | 8 |
| 26 | RYYWY | 105 | EINPSNGGTNFNEKLKS | 72 | EDDYDGTPDAMDY | 4 | RASQSVSSWLS | 133 | RASNRHT | 138 | QGHSYPYT | 8 |
| 27 | RYYLY | 2 | EINPSNGGTNFNESLKS | 110 | EDDYDGTPDAMDY | 53 | RASQSVSSWLS | 133 | RASNRHT | 138 | QGHSYPYT | 8 |
| 28 | RYYWY | 105 | EINPSNGGTNFNESLKS | 110 | EDDYDGTPDAMDY | 53 | RASQSVSSWLS | 133 | RASNRHT | 138 | QGHSYPYT | 8 |

TABLE 7
CDR Sequences of humanized EvB#52 (CDRH/CDRL sets)

| No. | CDRH1 | SEQ | CDRH2 | SEQ | CDRH3 | SEQ | CDRL1 | SEQ | CDRL2 | SEQ | CDRL3 | SEQ |
|---|---|---|---|---|---|---|---|---|---|---|---|---|
| 29 (#52 | RYYLY | | EINPSNGGTNFNEKFKS | 44 | EDDYDGTPDAMDY | 4, 53 | HASQNINVWLS | | RASNLHT | 7 | QGHSYPYT | 8 |
| murine | | | | | | | | | | | | |
| 30 | RYYMY | 67 | EINPSNGGTNFNEKFKS | 44 | EDDYDGTPDAMDY | 4 | HASQNINVWLS | 75 | RASNLHT | 7 | QGHSYPYT | 8 |
| 31 | RYYMY | 67 | EINPSNGGTNFAQKFQG | 68 | EDDYDGTPDAMDY | 4 | HASQNINVWLS | 75 | RASNLHT | 7 | QGHSYPYT | 8 |
| 32 | RYYLY | 2 | EINPSNGGTNFNEKLKS | 72 | EDDYDGTPDAMDY | 4 | HASQNINVWLS | 75 | RASNLHT | 7 | QGHSYPYT | 8 |
| 33 | RYYWY | 105 | EINPSNGGTNFNEKLKS | 72 | EDDYDGTPDAMDY | 4 | HASQNINVWLS | 75 | RASNLHT | 7 | QGHSYPYT | 8 |
| 34 | RYYLY | 2 | EINPSNGGTNFNESLKS | 110 | EDDYDGTPDAMDY | 4 | HASQNINVWLS | 75 | RASNLHT | 7 | QGHSYPYT | 8 |
| 35 | RYYWY | 105 | EINPSNGGTNFNESLKS | 110 | EDDYDGTPDAMDY | 4 | HASQNINVWLS | 75 | RASNLHT | 7 | QGHSYPYT | 8 |
| 36 | RYYLY | 2 | EINPSNGGTNFNEKFKS | 44 | EDDYDGTPDAMDY | 4 | RASQGISVWLS | 140 | RASNLHT | 7 | QGHSYPYT | 8 |
| 37 | RYYMY | 67 | EINPSNGGTNFNEKFKS | 44 | EDDYDGTPDAMDY | 4 | RASQGISVWLS | 140 | RASNLHT | 7 | QGHSYPYT | 8 |
| 38 | RYYMY | 67 | EINPSNGGTNFAQKFQG | 68 | EDDYDGTPDAMDY | 4 | RASQGISVWLS | 140 | RASNLHT | 7 | QGHSYPYT | 8 |
| 39 | RYYLY | 2 | EINPSNGGTNFNEKLKS | 72 | EDDYDGTPDAMDY | 4 | RASQGISVWLS | 140 | RASNLHT | 7 | QGHSYPYT | 8 |
| 40 | RYYWY | 105 | EINPSNGGTNFNEKLKS | 72 | EDDYDGTPDAMDY | 4 | RASQGISVWLS | 140 | RASNLHT | 7 | QGHSYPYT | 8 |
| 41 | RYYLY | 2 | EINPSNGGTNFNESLKS | 110 | EDDYDGTPDAMDY | 4 | RASQGISVWLS | 140 | RASNLHT | 7 | QGHSYPYT | 8 |
| 42 | RYYWY | 105 | EINPSNGGTNFNESLKS | 110 | EDDYDGTPDAMDY | 4 | RASQGISVWLS | 140 | RASNLHT | 7 | QGHSYPYT | 8 |
| 43 | RYYLY | 2 | EINPSNGGTNFNEKFKS | 44 | EDDYDGTPDAMDY | 4 | RASQSVSVWLS | 141 | RASNLHT | 7 | QGHSYPYT | 8 |
| 44 | RYYMY | 67 | EINPSNGGTNFNEKFKS | 44 | EDDYDGTPDAMDY | 4 | RASQSVSVWLS | 141 | RASNLHT | 7 | QGHSYPYT | 8 |
| 45 | RYYMY | 57 | EINPSNGGTNFAQKFQG | 68 | EDDYDGTPDAMDY | 4 | RASQSVSVWLS | 141 | RASNLHT | 7 | QGHSYPYT | 8 |
| 46 | RYYLY | 2 | EINPSNGGTNFNEKLKS | 72 | EDDYDGTPDAMDY | 4 | RASQSVSVWLS | 141 | RASNLHT | 7 | QGHSYPYT | 8 |
| 47 | RYYWY | 105 | EINPSNGGTNFNEKLKS | 72 | EDDYDGTPDAMDY | 4 | RASQSVSVWLS | 141 | RASNLHT | 7 | QGHSYPYT | 8 |
| 48 | RYYLY | 2 | EINPSNGGTNFNESLKS | 110 | EDDYDGTPDAMDY | 4 | RASQSVSVWLS | 141 | RASNLHT | 7 | QGHSYPYT | 8 |
| 49 | RYYWY | 105 | EINPSNGGTNFNESLKS | 110 | EDDYDGTPDAMDY | 1 | RASQSVSVWLS | 141 | RASNLHT | 7 | QGHSYPYT | 8 |
| 50 | RYYLY | 2 | EINPSNGGTNFNEKFKS | 44 | EDDYDGTPDAMDY | 4 | RASQSVSVWLS | 141 | RASNRHT | 138 | QGHSYPYT | 8 |
| 51 | RYYMY | 67 | EINPSNGGTNFNEKFKS | 44 | EDDYDGTPDAMDY | 4 | RASQSVSVWLS | 141 | RASNRHT | 138 | QGHSYPYT | 8 |
| 52 | RYYMY | 67 | EINPSNGGTNFAQKFQG | 68 | EDDYDGTPDAMDY | 4 | RASQSVSVWLS | 141 | RASNRHT | 138 | QGHSYPYT | 8 |
| 53 | RYYLY | 2 | EINPSNGGTNFNEKLKS | 72 | EDDYDGTPDAMDY | 4 | RASQSVSVWLS | 141 | RASNRHT | 138 | QGHSYPYT | 8 |
| 54 | RYYWY | 105 | EINPSNGGTNFNEKLKS | 72 | EDDYDGTPDAMDY | 4 | RASQSVSVWLS | 141 | RASNRHT | 138 | QGHSYPYT | 8 |
| 55 | RYYLY | 2 | EINPSNGGTNFNESLKS | 110 | EDDYDGTPDAMDY | 4 | RASQSVSVWLS | 141 | RASNRHT | 138 | QGHSYPYT | 8 |
| 56 | RYYWY | 105 | EINPSNGGTNFNESLKS | 110 | EDDYDGTPDAMDY | 4 | RASQSVSVWLS | 141 | RASNRHT | 138 | QGHSYPYT | 8 |

1. A bispecific antibody comprising a first binding part specifically and agonistically binding to human CD277 and a second binding part specifically binding to a tumor-antigen, characterized in that said first binding part is a full-length bivalent antibody and said second binding part consists of two identical single-chain Fv antibodies specifically binding to said tumor-antigen each of said single-chain Fv antibodies is linked by a peptide linker to each C-terminus of the first binding part.

2. The bispecific antibody according to claim 1, characterized in that each of said single-chain Fv antibodies is linked by a peptide linker with its N-terminus of the variable light chain to each C-terminus of the first binding part

3. The bispecific antibody according to claim 1, characterized in that said first binding part comprises as heavy chain CDR sequences CDRH1 of SEQ ID NO:43, CDRH2 of SEQ ID NO:44, and CDRH3 of SEQ ID NO:45 and as light chain CDR sequences CDRL1 of SEQ ID NO:6, CDRL2 of SEQ ID NO:7, and CDRL3 of SEQ ID NO:8.

4. The bispecific antibody according to claim 3, characterized in that CDRH2 of SEQ ID NO:44 is replaced by SEQ ID NO:68, SEQ ID NO:72, or SEQ ID NO:110.

5. The bispecific antibody according to claim 3, characterized in that CDRL1 of SEQ ID NO:6 is replaced by SEQ ID NO:75, SEQ ID NO:121, SEQ ID NO:133, SEQ ID NO:140 or SEQ ID NO:141.

6. The bispecific antibody according to claim 1, characterized in comprising as heavy chain CDR sequences CDRH1 of SEQ ID NO:43, CDRH2 of SEQ ID NO:44, and CDRH3 of SEQ ID NO:45, and
b) as light chain CDR sequences a CDR set selected from the group consisting of
b1) CDRL1 of SEQ ID NO:75, CDRL2 of SEQ ID NO:76, and CDRL3 of SEQ ID NO:77,
b2) CDRL1 of SEQ ID NO:79, CDRL2 of SEQ ID NO:80, and CDRL3 of SEQ ID NO:81,
b3) CDRL1 of SEQ ID NO:83, CDRL2 of SEQ ID NO:84, and CDRL3 of SEQ ID NO:85,
b4) CDRL1 of SEQ ID NO:87, CDRL2 of SEQ ID NO:88, and CDRL3 of SEQ ID NO:89,
b5) CDRL1 of SEQ ID NO:117, CDRL2 of SEQ ID NO:118, and CDRL3 of SEQ ID NO:119,
b6) CDRL1 of SEQ ID NO:121, CDRL2 of SEQ ID NO:122, and CDRL3 of SEQ ID NO:123,
b7) CDRL1 of SEQ ID NO:125, CDRL2 of SEQ ID NO:126, and CDRL3 of SEQ ID NO:127,
b8) CDRL1 of SEQ ID NO:129, CDRL2 of SEQ ID NO:130, and CDRL3 of SEQ ID NO:131,
b9) CDRL1 of SEQ ID NO:133, CDRL2 of SEQ ID NO:134, and CDRL3 of SEQ ID NO:135,
b10) CDRL1 of SEQ ID NO:137, CDRL2 of SEQ ID NO:138, and CDRL3 of SEQ ID NO:139,
b11) CDRL1 of SEQ ID NO:133, CDRL2 of SEQ ID NO:138, and CDRL3 of SEQ ID NO:139,
b12) CDRL1 of SEQ ID NO:140, CDRL2 of SEQ ID NO:134, and CDRL3 of SEQ ID NO:135,
b13) CDRL1 of SEQ ID NO:141, CDRL2 of SEQ ID NO:134, and CDRL3 of SEQ ID NO:135,
b14) CDRL1 of SEQ ID NO:141, CDRL2 of SEQ ID NO:138, and CDRL3 of SEQ ID NO:135,
b15) CDRL1 of SEQ ID NO:151, CDRL2 of SEQ ID NO:7, and CDRL3 of SEQ ID NO:8,
b16) CDRL1 of SEQ ID NO:152, CDRL2 of SEQ ID NO:7, and CDRL3 of SEQ ID NO:8,
b17) CDRL1 of SEQ ID NO:153, CDRL2 of SEQ ID NO:7, and CDRL3 of SEQ ID NO:8,
b18) CDRL1 of SEQ ID NO:6, CDRL2 of SEQ ID NO:7, and CDRL3 of SEQ ID NO:156,
b19) CDRL1 of SEQ ID NO:6, CDRL2 of SEQ ID NO:7, and CDRL3 of SEQ ID NO:157,
b20) CDRL1 of SEQ ID NO:6, CDRL2 of SEQ ID NO:7, and CDRL3 of SEQ ID NO:158,
b21) CDRL1 of SEQ ID NO:154, CDRL2 of SEQ ID NO:7, and CDRL3 of SEQ ID NO:8, and
b22) CDRL1 of SEQ ID NO:155, CDRL2 of SEQ ID NO:7, and CDRL3 of SEQ ID NO:8.

7. The bispecific antibody according to claim 1, characterized in that said tumor-antigen is selected from the group consisting of CLDN18.2, FOLR1, STEAP1, and DLL3.

8. The bispecific antibody according to claim 7, characterized in that for said second binding part the variable light and heavy chain CDRs are
a) CDRL1 of SEQ ID NO:11, CDRL2 of SEQ ID NO:12, and CDRL3 of SEQ ID NO:13 and the variable heavy chain CDRs are CDRH1 of SEQ ID NO:15, CDRH2 of SEQ ID NO:16, and CDRH3 of SEQ ID NO:17 for FOLR1 as tumor-antigen,
b) CDRL1 of SEQ ID NO:19, CDRL2 of SEQ ID NO:20, and CDRL3 of SEQ ID NO:21 and CDRH1 of SEQ ID NO:23, CDRH2 of SEQ ID NO:24, and CDRH3 of SEQ ID NO:25 for STEAP1 as tumor-antigen,
c) CDRL1 of SEQ ID NO:27, CDRL2 of SEQ ID NO:28, and CDRL3 of SEQ ID NO:29 and CDRH1 of SEQ ID NO:31, CDRH2 of SEQ ID NO:32, and CDRH3 of SEQ ID NO:33 for DLL3 as tumor-antigen, or
d) CDRL1 of SEQ ID NO:35, CDRL2 of SEQ ID NO:36, and CDRL3 of SEQ ID NO:37 and CDRH1 of SEQ ID NO:39, CDRH2 of SEQ ID NO:40, and CDRH3 of SEQ ID NO:41 for CLDN18.2 as tumor-antigen.

9. The bispecific antibody according to claim 1, characterized in that for the first binding part the variable heavy chain is of SEQ ID NO:42 and the variable light chain is selected from the group consisting of SEQ ID NO:5, SEQ ID NO:74, SEQ ID NO:78, SEQ ID NO:82, and SEQ ID NO:86.

10. The bispecific antibody according to claim 9, characterized in that for said second binding part the variable light chain is of SEQ ID NO:10 and the variable heavy chain is of SEQ ID NO:14.

11. The bispecific antibody according to claim 1, characterized in that the first binding part of the antibody according to the invention is a humanized or CDR grafted antibody.

12. The bispecific antibody according to claim 1, characterized in that said scFvs are bound to said C-termini in the orientation peptidelinker1-VL-peptidelinker2-VH.

13. The bispecific antibody according to claim 12, characterized in that said first peptide-linker consists of 5-25 amino acids and said second peptide-linker consists of 10-25 amino acids.

14. The bispecific antibody according to claim 1, for use in the treatment of a tumor disease.

15. The bispecific antibody according to claim 1, for use in the treatment of a tumor disease, selected from the group consisting of colon carcinoma, ovarian cancer, lung cancer, prostate cancer, pancreatic cancer, and breast cancer.

16. A pharmaceutical composition, comprising a bispecific antibody according to claim 1.

17. A method for treating of cancer in an individual, comprising administering to the individual an effective amount of a bispecific antibody according to claim 1.

18. A recombinant nucleic acid sequence encoding a bispecific antibody according to claim 1.
